SEQUENCE LISTING (125 sequences)

SEQ ID NO: 1 - protein, 247 aa, Artificial sequence
  scFv inhibitor
EVQLQQSGPELVKPGASVKMSCKASGYTFTDYYIKWVKQSHGKSLEWIGVINPGSGGTNYNEKFKGKATLTADKSSSTAYMQLSSLTSENSAVYFCARRAGTGYYFDYWGQGTTVTVSSGGGGSGGGGSGGGGSDVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPLTFGAGTKLEIKR

SEQ ID NO: 2 - protein, 243 aa, Artificial sequence
  scFv inhibitor
EVQLQQSGAELARPGASVKLSCKASGYTFTSYWMQWMKQRPGQGLEWIGAIYPGDGDTRYTQKFKGKATLTADKSSSTAYMQLSSLASEDSAVYYCARSRAMDDWGQGTSLTVSSGGGGSGGGGSGGGGSDVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPRTFGGGTKLEIKR

SEQ ID NO: 3 - protein, 248 aa, Artificial sequence
  scFv inhibitor
QVQLQQSGPELVKPGASVKLSCKTSGFTFSSTFFSWLKQKPGQSLEWIAWIYAGSGGSSYNQKFTGKAQLTVDTSSSTAYIQFSSLTTEDSAIYYCARQNLDYDYAMDYWGQGTSVTVSSGGGGSGGGGSGGGGSDVLMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPYTFGGGTKLEIKR

SEQ ID NO: 4 - protein, 246 aa, Artificial sequence
  scFv inhibitor
EVQLQQSGAELVRSGASVKLSCTASGFNIKDYYMHWVKQRPEQGLEWIGWIDPENGDTEYTQKFKGKATLTADKSPSTAYMQLSSLTSEDSAVYYCARITTDYYFDYWGQGTTLTVSSGGGGSGGGGSGGGGSDVVMTQTPLSLPVSLGDQASISCRSSQSLVHSNGNTYLEWYLQKPGQSPKLLIYKVSNRFSGVPDRFSGSGSGTDFTLKISRVEAEDLGVYFCSQSTHVPLTFGAGTKLEIKR

SEQ ID NO: 5 - protein, 244 aa, Artificial sequence
  scFv inhibitor
EVQLQQSGAELVRSGASVKLSCTASGFNIKDYYMHWVKQRPEQGLEWIGWIDPANGNTKYDPKFQGKATITADTSSNTAYLQLSSLTSEDTAVYYCARWDWYFDVWGAGTTVTVSSGGGGSGGGGSGGGGSSVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPWTFGGGTKLELKR

SEQ ID NO: 6 - protein, 249 aa, Artificial sequence
  scFv inhibitor
EVQLQQSGAELVRSGASVKLSCTASGFNIKDYYMHWVKQRPEQGLEWIGWIDPENGDTEYAPKFQGKATMTADTSSNTAYLQLSSLTSEDTAVYYCNAWGGSSDYYAMDYWGQGTSVTVSSGGGGSGGGGSGGGGSDVVMTQTPLSLPVSLGDQASISCRSSQSLVHSNGNTYLHWYLQKPGQSPKLLIYKVSNRFSGVPDRFSGSGSGTDFTLKISRVEAEDLGVYYCFQGSHVPWTFGGGTKLEIKR

SEQ ID NO: 7 - protein, 246 aa, Artificial sequence
  scFv inhibitor
EIQLQQSGAELVKPGASVKLSCTASGFNIKDTFVHWVKQRPEQGLEWIGRIDPANGNTKYDPKFQGKATITAATSSNTAYLQLSSLTSEDTAVYYCATGLDYYAMDYWGQGTSVTVSSGGGGSGGGGSGGGGSDVVMTQTPLSPPVSLGDQASISCRSSQSLVHSNGNTYLHWYLQKPGQSPKLLIYKVSNRFSGVPDRFSGSGSGTDFTLKITRVEAEDLGLYFCSQSTHVPWTFGGGTKLEIKR

SEQ ID NO: 8 - protein, 249 aa, Artificial sequence
  scFv inhibitor
EVQLQQSGTELVKPGASVKLSCATSGFNIKDTYMQWVKQRPEQGLEWIGWIDPENGDTESAPKFQGKATMTADTSSNTAYLQLSSLTSEDSAVYFCARERDYYGYEGFAYWGQGTLVTVSSGGGGSGGGGSGGGGSDVVMTQTPLSLPVSLGDQASISCRSSQSLVHSNGNTYLEWYLQKPGQSPKLLIYKVSNRFSGVPDRFSGSGSGTDFTLKISRVEAEDLGVYFCSQSTHVPLTFGAGTKLEIKR

SEQ ID NO: 9 - protein, 240 aa, Artificial sequence
  scFv inhibitor
QVQLQQPGAELVKPGASVKLSCTASGFNIKDTYMHWVKQRPEQGLEWIGRIDPANGNTKYDPKFQGKATITADKSSSTAYMQLSSLTSEDSAVYYCTRIWLPWGQGTLVTVSSGGGGSGGGGSGGGGSDVLMTQTPLSLPDSLGDQASISCRSSQSIVHSNGNTYLEWYLQKPGQSPKLLIYKVSNRFSGVPDRFSGSGSGTDFTLKISRVEAEDLGVYFCSQSTHVLTFGAGTKLELKR

SEQ ID NO: 10 - protein, 242 aa, Artificial sequence
  scFv inhibitor
EVQLQQSGAELVKPGASVKLSCTASGFNIKDYYMHWVKQRPGQGLEYIGRIYPGNGQTDYNGKFKGKATLTADKSSSTAYMQLSSLTSVDSAVYFCARGLFAYWGQGTLVTVSSGGGGSGGGGSGGGGSDVVMTQTPLSLPVSFGDQVSISCRSSQSLANSYGNTYLSWCLHKPGQSPQLLIYGISNRFSGVPDRFSGSGSGTDFTLKISTIKPEDLGMYYCLQGTHQPLTFGAGTKLEIKR

SEQ ID NO: 11 - protein, 245 aa, Artificial sequence
  scFv inhibitor
QVQLQQSGAELMKPGASVKISCKATGYTFSNYWIEWVKQRPGHGLEWIGGILPGSGSTNYNEKFKGKATFTADTSSNTAYLQLSSLTSEDTAVYYCNADGPPGFDYWGQGTTLTVSSGGGGSGGGGSGGGGSDVLMTQTPLTLSVTIGQPASISCKSSQSLLDSDGKTYLNWLLQRPGQSPKRLIYLVSKLDSGVPDRFTGSGSGTDFTLKISRVEAEDLGVYYCFQGSHVPFTFGSGTKLEIKR

SEQ ID NO: 12 - protein, 114 aa, Artificial sequence
  dAb inhibitor
EVQLQQSGAELVKPGASVKLSCTASGFNIKDYYMHWVKQRPGQGLEYIGRIYPGNGQTDYNGKFKGKATLTADKSSSTAYMQLSSLTSVDSAVYFCARGLFAYWGQGTLVTVSS

SEQ ID NO: 13 - protein, 118 aa, Artificial sequence
  dAb inhibitor
EIQLQQSGAELVKPGASVKLSCTASGFNIKDTFVHWVKQRPEQGLEWIGRIDPANGNTKYDPKFQGKATITAATSSNTAYLQLSSLTSEDTAVYYCATGLDYYAMDYWGQGTSVTVSS

SEQ ID NO: 14 - protein, 116 aa, Artificial sequence
  dAb inhibitor
EVQLQQSGAELVRSGASVKLSCTASGFNIKDYYMHWVKQRPEQGLEWIGWIDPANGNTKYDPKFQGKATITADTSSNTAYLQLSSLTSEDTAVYYCARWDWYFDVWGAGTTVTVSS

SEQ ID NO: 15 - protein, 636 aa, Artificial sequence
  MBP-scFv inhibitor
MAMKTEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSVPSSLVIEGRPEFSTMEVQLQQSGPELVKPGASVKMSCKASGYTFTDYYIKWVKQSHGKSLEWIGVINPGSGGTNYNEKFKGKATLTADKSSSTAYMQLSSLTSENSAVYFCARRAGTGYYFDYWGQGTTVTVSSGGGGSGGGGSGGGGSDVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPLTFGAGTKLEIKR

SEQ ID NO: 16 - protein, 632 aa, Artificial sequence
  MBP-scFv inhibitor
MAMKTEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSVPSSLVIEGRPEFSTMEVQLQQSGAELARPGASVKLSCKASGYTFTSYWMQWMKQRPGQGLEWIGAIYPGDGDTRYTQKFKGKATLTADKSSSTAYMQLSSLASEDSAVYYCARSRAMDDWGQGTSLTVSSGGGGSGGGGSGGGGSDVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPRTFGGGTKLEIKR

SEQ ID NO: 17 - protein, 637 aa, Artificial sequence
  MBP-scFv inhibitor
MAMKTEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSVPSSLVIEGRPEFSTMQVQLQQSGPELVKPGASVKLSCKTSGFTFSSTFFSWLKQKPGQSLEWIAWIYAGSGGSSYNQKFTGKAQLTVDTSSSTAYIQFSSLTTEDSAIYYCARQNLDYDYAMDYWGQGTSVTVSSGGGGSGGGGSGGGGSDVLMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPYTFGGGTKLEIKR

SEQ ID NO: 18 - protein, 635 aa, Artificial sequence
  MBP-scFv inhibitor
MAMKTEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSVPSSLVIEGRPEFSTMEVQLQQSGAELVRSGASVKLSCTASGFNIKDYYMHWVKQRPEQGLEWIGWIDPENGDTEYTQKFKGKATLTADKSPSTAYMQLSSLTSEDSAVYYCARITTDYYFDYWGQGTTLTVSSGGGGSGGGGSGGGGSDVVMTQTPLSLPVSLGDQASISCRSSQSLVHSNGNTYLEWYLQKPGQSPKLLIYKVSNRFSGVPDRFSGSGSGTDFTLKISRVEAEDLGVYFCSQSTHVPLTFGAGTKLEIKR

SEQ ID NO: 19 - protein, 633 aa, Artificial sequence
  MBP-scFv inhibitor
MAMKTEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSVPSSLVIEGRPEFSTMEVQLQQSGAELVRSGASVKLSCTASGFNIKDYYMHWVKQRPEQGLEWIGWIDPANGNTKYDPKFQGKATITADTSSNTAYLQLSSLTSEDTAVYYCARWDWYFDVWGAGTTVTVSSGGGGSGGGGSGGGGSSVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPWTFGGGTKLELKR

SEQ ID NO: 20 - protein, 638 aa, Artificial sequence
  MBP-scFv inhibitor
MAMKTEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSVPSSLVIEGRPEFSTMEVQLQQSGAELVRSGASVKLSCTASGFNIKDYYMHWVKQRPEQGLEWIGWIDPENGDTEYAPKFQGKATMTADTSSNTAYLQLSSLTSEDTAVYYCNAWGGSSDYYAMDYWGQGTSVTVSSGGGGSGGGGSGGGGSDVVMTQTPLSLPVSLGDQASISCRSSQSLVHSNGNTYLHWYLQKPGQSPKLLIYKVSNRFSGVPDRFSGSGSGTDFTLKISRVEAEDLGVYYCFQGSHVPWTFGGGTKLEIKR

SEQ ID NO: 21 - protein, 635 aa, Artificial sequence
  MBP-scFv inhibitor
MAMKTEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSVPSSLVIEGRPEFSTMEIQLQQSGAELVKPGASVKLSCTASGFNIKDTFVHWVKQRPEQGLEWIGRIDPANGNTKYDPKFQGKATITAATSSNTAYLQLSSLTSEDTAVYYCATGLDYYAMDYWGQGTSVTVSSGGGGSGGGGSGGGGSDVVMTQTPLSPPVSLGDQASISCRSSQSLVHSNGNTYLHWYLQKPGQSPKLLIYKVSNRFSGVPDRFSGSGSGTDFTLKITRVEAEDLGLYFCSQSTHVPWTFGGGTKLEIKR

SEQ ID NO: 22 - protein, 638 aa, Artificial sequence
  MBP-scFv inhibitor
MAMKTEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSVPSSLVIEGRPEFSTMEVQLQQSGTELVKPGASVKLSCATSGFNIKDTYMQWVKQRPEQGLEWIGWIDPENGDTESAPKFQGKATMTADTSSNTAYLQLSSLTSEDSAVYFCARERDYYGYEGFAYWGQGTLVTVSSGGGGSGGGGSGGGGSDVVMTQTPLSLPVSLGDQASISCRSSQSLVHSNGNTYLEWYLQKPGQSPKLLIYKVSNRFSGVPDRFSGSGSGTDFTLKISRVEAEDLGVYFCSQSTHVPLTFGAGTKLEIKR

SEQ ID NO: 23 - protein, 629 aa, Artificial sequence
  MBP-scFv inhibitor
MAMKTEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSVPSSLVIEGRPEFSTMQVQLQQPGAELVKPGASVKLSCTASGFNIKDTYMHWVKQRPEQGLEWIGRIDPANGNTKYDPKFQGKATITADKSSSTAYMQLSSLTSEDSAVYYCTRIWLPWGQGTLVTVSSGGGGSGGGGSGGGGSDVLMTQTPLSLPDSLGDQASISCRSSQSIVHSNGNTYLEWYLQKPGQSPKLLIYKVSNRFSGVPDRFSGSGSGTDFTLKISRVEAEDLGVYFCSQSTHVLTFGAGTKLELKR

SEQ ID NO: 24 - protein, 631 aa, Artificial sequence
  MBP-scFv inhibitor
MAMKTEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSVPSSLVIEGRPEFSTMEVQLQQSGAELVKPGASVKLSCTASGFNIKDYYMHWVKQRPGQGLEYIGRIYPGNGQTDYNGKFKGKATLTADKSSSTAYMQLSSLTSVDSAVYFCARGLFAYWGQGTLVTVSSGGGGSGGGGSGGGGSDVVMTQTPLSLPVSFGDQVSISCRSSQSLANSYGNTYLSWCLHKPGQSPQLLIYGISNRFSGVPDRFSGSGSGTDFTLKISTIKPEDLGMYYCLQGTHQPLTFGAGTKLEIKR

SEQ ID NO: 25 - protein, 634 aa, Artificial sequence
  MBP-scFv inhibitor
MAMKTEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSVPSSLVIEGRPEFSTMQVQLQQSGAELMKPGASVKISCKATGYTFSNYWIEWVKQRPGHGLEWIGGILPGSGSTNYNEKFKGKATFTADTSSNTAYLQLSSLTSEDTAVYYCNADGPPGFDYWGQGTTLTVSSGGGGSGGGGSGGGGSDVLMTQTPLTLSVTIGQPASISCKSSQSLLDSDGKTYLNWLLQRPGQSPKRLIYLVSKLDSGVPDRFTGSGSGTDFTLKISRVEAEDLGVYYCFQGSHVPFTFGSGTKLEIKR

SEQ ID NO: 26 - protein, 503 aa, Artificial sequence
  MBP-dAb inhibitor
MAMKTEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSVPSSLVIEGRPEFSTMEVQLQQSGAELVKPGASVKLSCTASGFNIKDYYMHWVKQRPGQGLEYIGRIYPGNGQTDYNGKFKGKATLTADKSSSTAYMQLSSLTSVDSAVYFCARGLFAYWGQGTLVTVSS

SEQ ID NO: 27 - protein, 507 aa, Artificial sequence
  MBP-dAb inhibitor
MAMKTEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSVPSSLVIEGRPEFSTMEIQLQQSGAELVKPGASVKLSCTASGFNIKDTFVHWVKQRPEQGLEWIGRIDPANGNTKYDPKFQGKATITAATSSNTAYLQLSSLTSEDTAVYYCATGLDYYAMDYWGQGTSVTVSS

SEQ ID NO: 28 - protein, 505 aa, Artificial sequence
  MBP-dAb inhibitor
MAMKTEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSVPSSLVIEGRPEFSTMEVQLQQSGAELVRSGASVKLSCTASGFNIKDYYMHWVKQRPEQGLEWIGWIDPANGNTKYDPKFQGKATITADTSSNTAYLQLSSLTSEDTAVYYCARWDWYFDVWGAGTTVTVSS

SEQ ID NO: 29 - protein, 8 aa, Artificial sequence
  Inhibitor peptide
LGLAVEVR

SEQ ID NO: 30 - protein, 8 aa, Artificial sequence
  Inhibitor peptide
FSRAEWFC

SEQ ID NO: 31 - protein, 8 aa, Artificial sequence
  Inhibitor peptide
GAALNTSS

SEQ ID NO: 32 - protein, 8 aa, Artificial sequence
  Inhibitor peptide
AVLAGLGV

SEQ ID NO: 33 - protein, 8 aa, Artificial sequence
  Inhibitor peptide
FWGGALWR

SEQ ID NO: 34 - protein, 8 aa, Artificial sequence
  Inhibitor peptide
CCAWLSIR

SEQ ID NO: 35 - protein, 8 aa, Artificial sequence
  Inhibitor peptide
LAAWFCLW

SEQ ID NO: 36 - protein, 392 aa, Artificial sequence
  Internal position inhibitor peptide aptamer
MHHHHHHSGKTEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPDPLGLAVEVRDPGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSVPDYKDDDDK

SEQ ID NO: 37 - protein, 392 aa, Artificial sequence
  Internal position inhibitor peptide aptamer
MHHHHHHSGKTEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPDPFSRAEWFCDPGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSVPDYKDDDDK

SEQ ID NO: 38 - protein, 392 aa, Artificial sequence
  Internal position inhibitor peptide aptamer
MHHHHHHSGKTEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPDPGAALNTSSDPGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSVPDYKDDDDK

SEQ ID NO: 39 - protein, 392 aa, Artificial sequence
  Internal position inhibitor peptide aptamer
MHHHHHHSGKTEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPDPAVLAGLGVDPGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSVPDYKDDDDK

SEQ ID NO: 40 - protein, 392 aa, Artificial sequence
  Internal position inhibitor peptide aptamer
MHHHHHHSGKTEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPDPFWGGALWRDPGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSVPDYKDDDDK

SEQ ID NO: 41 - protein, 392 aa, Artificial sequence
  Internal position inhibitor peptide aptamer
MHHHHHHSGKTEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPDPCCAWLSIRDPGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSVPDYKDDDDK

SEQ ID NO: 42 - protein, 392 aa, Artificial sequence
  Internal position inhibitor peptide aptamer
MHHHHHHSGKTEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPDPLAAWFCLWDPGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSVPDYKDDDDK

SEQ ID NO: 43 - protein, 389 aa, Artificial sequence
  C′ fusion inhibitor peptide aptamer
MHHHHHHSGKTEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSVPLGLAVEVR

SEQ ID NO: 44 - protein, 389 aa, Artificial sequence
  C′ fusion inhibitor peptide aptamer
MHHHHHHSGKTEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSVPFSRAEWFC

SEQ ID NO: 45 - protein, 389 aa, Artificial sequence
  C′ fusion inhibitor peptide aptamer
MHHHHHHSGKTEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSVPGAALNTSS

SEQ ID NO: 46 - protein, 389 aa, Artificial sequence
  C′ fusion inhibitor peptide aptamer
MHHHHHHSGKTEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSVPAVLAGLGV

SEQ ID NO: 47 - protein, 389 aa, Artificial sequence
  C′ fusion inhibitor peptide aptamer
MHHHHHHSGKTEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSVPFWGGALWR

SEQ ID NO: 48 - protein, 389 aa, Artificial sequence
  C′ fusion inhibitor peptide aptamer
MHHHHHHSGKTEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSVPCCAWLSIR

SEQ ID NO: 49 - protein, 389 aa, Artificial sequence
  C′ fusion inhibitor peptide aptamer
MHHHHHHSGKTEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSVPLAAWFCLW

SEQ ID NO: 50 - protein, 9 aa, Artificial sequence
  inhibitor peptide
EDVVCCWPV

SEQ ID NO: 51 - protein, 9 aa, Artificial sequence
  inhibitor peptide
EDVVCCCLF

SEQ ID NO: 52 - protein, 9 aa, Artificial sequence
  inhibitor peptide
EAVVCCLSL

SEQ ID NO: 53 - protein, 9 aa, Artificial sequence
  inhibitor peptide
EDVVCCCGC

SEQ ID NO: 54 - protein, 9 aa, Artificial sequence
  inhibitor peptide
EDVVCCVLY

SEQ ID NO: 55 - protein, 390 aa, Artificial sequence
  C′ fusion inhibitor peptide aptamer
MHHHHHHSGKTEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSVPEDVVCCWPV

SEQ ID NO: 56 - protein, 390 aa, Artificial sequence
  C′ fusion inhibitor peptide aptamer
MHHHHHHSGKTEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSVPEDVVCCCLF

SEQ ID NO: 57 - protein, 390 aa, Artificial sequence
  C′ fusion inhibitor peptide aptamer
MHHHHHHSGKTEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSVPEAVVCCLSL

SEQ ID NO: 58 - protein, 390 aa, Artificial sequence
  C′ fusion inhibitor peptide aptamer
MHHHHHHSGKTEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSVPEDVVCCCGC

SEQ ID NO: 59 - protein, 390 aa, Artificial sequence
  C′ fusion inhibitor peptide aptamer
MHHHHHHSGKTEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSVPEDVVCCVLY

SEQ ID NO: 60 - protein, 10 aa, Artificial sequence
  NS3 cleavage site
EDVVCCSMSY

SEQ ID NO: 61 - protein, 10 aa, Artificial sequence
  NS3 cleavage site
DLEVVTSTWV

SEQ ID NO: 62 - protein, 10 aa, Artificial sequence
  NS3 cleavage site
DEMEECASHL

SEQ ID NO: 63 - protein, 10 aa, Artificial sequence
  NS3 cleavage site
DCSTPCSGSW

SEQ ID NO: 64 - protein, 1115 aa, Artificial sequence
  Reporter protein
MAQQSPYSAAMAEQRHQEWLRFVDLLKNAYQNDLHLPLLNLMLTPDEREALGTRVRIVEELLRGEMSQRELKNELGAGIADPVVLQRRDWENPGVTQLNRLAAHPPFASWRNSEEARTDRPSQQLRSLNGEWRFAWFPAPEAVPESWLECDLPEADTVVVPSNWQMHGYDAPIYTNVTYPITVNPPFVPTENPTGCYSLTFNVDESWLQEGQTRIIFDGVNSAFHLWCNGRWVGYGQDSRLPSEFDLSAFLRAGENRLAVMVLRWSDGSYLEDQDMWRMSGIFRDVSLLHKPTTQISDFHVATRFNDDFSRAVLEAEVQMCGELRDYLRVTVSLWQGETQVASGTAPFGGEIIDEGQRASEDVVCCSMSYTGARGGYADRVTLRLNVENPKLWSAEIPNLYRAVVELHTADGTLIEAEACDVGFREVRIENGLLLLNGKPLLIRGVNRHEHHPLHGQVMDEQTMVQDILLMKQNNFNAVRCSHYPNHPLWYTLCDRYGLYVVDEANIETHGMVPMNRLTDDPRWLPAMSERVTRMVQRDRNHPSVIIWSLGNESGHGANHDALYRWIKSVDPSRPVQYEGGGADTTATDIICPMYARVDEDQPFPAVPKWSIKKWLSLPGETRPLILCEYAHAMGNSLGGFAKYWQAFRQYPRLQGGFVWDWVDQSLIKYDENGNPWSAYGGDFGDTPNDRQFCMNGLVFADRTPHPALTEAKHQQQFFQFRLSGQTIEVTSEYLFRHSDNELLHWMVALDGKPLASGEVPLDVAPQGKQLIELPELPQPESAGQLWLTVRVVQPNATAWSEAGHISAWQQWRLAENLSVTLPAASHAIPHLTTSEMDFCIELGNKRWQFNRQSGFLSQMWIGDKKQLLTPLRDQFTRAPLDNDIGVSEATRIDPNAWVERWKAAGHYQAEAALLQCTADTLADAVLITTAHAWQHQGKTLFISRKTYRIDGSGQMAITVDVEVASDTPHPARIGLNCQLAQVAERVNWLGLGPQENYPDRLTAACFDRWDLPLSDMYTPYVFPSENGLRCGTRELNYGPHQWRGDFQFNISRYSQQQLMETSHRHLLHAEEGTWLNIDGFHMGIGGDDSWSPSVSAEFQLSAGRYHYQLVWCQK

SEQ ID NO: 65 - protein, 1033 aa, Artificial sequence
  Reporter protein
VVLQRRDWENPGVTQLNRLAAHPPFASWRNSEEARTDRPSQQLRSLNGEWRFAWFPAPEAVPESWLECDLPEADTVVVPSNWQMHGYDAPIYTNVTYPITVNPPFVPTENPTGCYSLTFNVDESWLQEGQTRIIFDGVNSAFHLWCNGRWVGYGQDSRLPSEFDLSAFLRAGENRLAVMVLRWSDGSYLEDQDMWRMSGIFRDVSLLHKPTTQISDFHVATRFNDDFSRAVLEAEVQMCGELRDYLRVTVSLWQGETQVASGTAPFGGEIIDEGQRASEDVVCCSMSYTGARGGYADRVTLRLNVENPKLWSAEIPNLYRAVVELHTADGTLIEAEACDVGFREVRIENGLLLLNGKPLLIRGVNRHEHHPLHGQVMDEQTMVQDILLMKQNNFNAVRCSHYPNHPLWYTLCDRYGLYVVDEANIETHGMVPMNRLTDDPRWLPAMSERVTRMVQRDRNHPSVIIWSLGNESGHGANHDALYRWIKSVDPSRPVQYEGGGADTTATDIICPMYARVDEDQPFPAVPKWSIKKWLSLPGETRPLILCEYAHAMGNSLGGFAKYWQAFRQYPRLQGGFVWDWVDQSLIKYDENGNPWSAYGGDFGDTPNDRQFCMNGLVFADRTPHPALTEAKHQQQFFQFRLSGQTIEVTSEYLFRHSDNELLHWMVALDGKPLASGEVPLDVAPQGKQLIELPELPQPESAGQLWLTVRVVQPNATAWSEAGHISAWQQWRLAENLSVTLPAASHAIPHLTTSEMDFCIELGNKRWQFNRQSGFLSQMWIGDKKQLLTPLRDQFTRAPLDNDIGVSEATRIDPNAWVERWKAAGHYQAEAALLQCTADTLADAVLITTAHAWQHQGKTLFISRKTYRIDGSGQMAITVDVEVASDTPHPARIGLNCQLAQVAERVNWLGLGPQENYPDRLTAACFDRWDLPLSDMYTPYVFPSENGLRCGTRELNYGPHQWRGDFQFNISRYSQQQLMETSHRHLLHAEEGTWLNIDGFHMGIGGDDSWSPSVSAEFQLSAGRYHYQLVWCQK

SEQ ID NO: 66 - protein, 587 aa, Artificial sequence
  Recombinant protease
MAMKTEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSVPSSLVIEGRPEFSTMAPIGSVVIVGRIILSGRGGPITAYSQQTRGLLGCIITSLTGRDKNQVEGEVQVVSTATQSFLATCVNGACWTVFHGAGSKTLAGPKGPITQMYTNVDQDLVGWPAPPGARSLTPCTCGSSDLYLVTRHADVIPVRRRGDTRGSLLSPRPISYLKGSSGGPLLCPSGHVVGIFRAAVCTRGVAKAVDFIPVESMETTMR

SEQ ID NO: 67 - DNA, 10905 bp, Artificial sequence
  pMGT14
tttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattttataaaccgtggagcgggcaatactgagctgatgagcaatttccgttgcaccagtgcccttctgatgaagcgtcagcacgacgttcctgtccacggtacgcctgcggccaaatttgattcctttcagctttgcttcctgtcggccctcattcgtgcgctctaggatcctccggcgttcagcctgtgccacagccgacaggatggtgaccaccatttgccccatatcaccgtcggtactgatcccgtcgtcaataaaccgaaccgctacaccctgagcatcaaactcttttatcagttggatcatgtcggcggtgtcgcggccaagacggtcgagcttcttcaccagaatgacatcaccttcctccaccttcatcctcagcaaatccagcccttcccgatctgttgaactgccggatgccttgtcggtaaagatgcggttagcttttacccctgcatctttgagcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaaggggtctgacgctcagtggaacgaaaactcacgttaagggattttggtcatgagattatcaaaaaggatcttcacctagatccttttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagcgcggccgcttaccgcatagtggtttccatagattcaacgggtatgaagtccaccgccttcgcgaccccccgggtgcacacggcagcccggaagatgcccacaacgtgccccgaggggcagagcaatggaccacccgaagagcccttcaagtaggagatgggtctaggggagagcaagctccccctagtgtcgccccgccggcgcaccgggatgacatcagcatgtctcgtgaccaggtaaaggtccgagctgccgcaggtgcacggtgttaaggaacgcgccccggggggcgctggccagccgacgaggtcctggtctacattggtgtacatttgggtgattgggccttttgggccggctaaggtctttgagccggcaccgtgaaagacagtccagcacgcgccgttgacacaggtcgccaggaaagattgtgttgcggtggagaccacttgaacctccccctcaacctggttcttgtcccggcccgtgagactagtgataatgcaaccaagtaggccccgcgtctgttgggagtaggccgtgatcgggccaccacggccggacaggatgattctgcctacgattactactgagccgataggcgccatggtactgaattcaggcctaccctcgatcacgagagaggacggtaccgagctcgaattagtctgcgcgtctttcagggcttcatcgacagtctgacgaccgctggcggcgttgatcaccgcagtacgcacggcataccagaaagcggacatctgcgggatgttcggcatgatttcacctttctgggcgttttccatagtggcggcaatacgtggatctttcgccaactcttcctcgtaagacttcagcgctacggcacccagcggtttgtctttattaaccgcttccagaccttcatcagtcagcagatagttttcgaggaactctttcgccagctctttgttcggactggcggcgttaatacctgcgctcagcacgccaacgaacggtttggatggttgacccttgaaggtcggcagtaccgttacaccataattcactttgctggtgtcgatgttggaccatgcccacgggccgttgatggtcatcgctgtttcgcctttattaaaggcagcttctgcgatggagtaatcggtgtctgcattcatgtgtttgtttttaatcaggtcaaccaggaaggtcagacccgctttcgcgccagcgttatccacgcccacgtctttaatgtcgtacttgccgttttcatacttgaacgcataacccccgtcagcagcaatcagcggccaggtgaagtacggttcttgcaggttgaacatcagcgcgctcttacctttcgctttcagttctttatccagcgccgggatctcttcccaggtttttggcgggttcggcagcagatctttgttataaatcagcgataacgcttcaacagcgatcgggtaagcaatcagcttgccgttgtaacgtacggcatcccaggtaaacggatacagcttgtcctggaacgctttgtccggggtgatttcagccaacaggccagattgagcgtagccaccaaagcggtcgtgtgcccagaagataatgtcagggccatcgccagttgccgcaacctgtgggaatttctcttccagtttatccggatgctcaacggtgactttaattccggtatctttctcgaatttcttaccgacttcagcgagaccgttatagcctttatcgccgttaatccagattaccagtttaccttcttcagttttcatggttaattcctcctgttagcccaaaaaacgggtatggagaaacagtagagagttgcgataaaaagcgtcaggtaggatccgctaatcttatggataaaaatgctatggcatagcaaagtgtgacgccgtgcaaataatcaatgtggacttttctgccgtgattatagacacttttgttacgcgtttttgtcatggctttggtcccgctttgttacagaatgcttttaataagcggggttaccggtttggttagcgagaagagccagtaaaagacgcagtgacggcaatgtctgatgcaatatggacaattggtttcttctctgaatggcgggagtatgaaaagtatggctgaagcgcaaaatgatcccctgctgccgggatactcgtttaatgcccatctggtggcgggtttaacgccgattgaggccaacggttatctcgatttttttatcgaccgaccgctgggaatgaaaggttatattctcaatctcaccattcgcggtcagggggtggtgaaaaatcagggacgagaatttgtttgccgaccgggtgatattttgctgttcccgccaggagagattcatcactacggtcgtcatccggaggctcgcgaatggtatcaccagtgggtttactttcgtccgcgcgcctactggcatgaatggcttaactggccgtcaatatttgccaatacggggttctttcgcccggatgaagcgcaccagccgcatttcagcgacctgtttgggcaaatcattaacgccgggcaaggggaagggcgctattcggagctgctggcgataaatctgcttgagcaattgttactgcggcgcatggaagcgattaacgagtcgctccatccaccgatggataatcgggtacgcgaggcttgtcagtacatcagcgatcacctggcagacagcaattttgatatcgccagcgtcgcacagcatgtttgcttgtcgccgtcgcgtctgtcacatcttttccgccagcagttagggattagcgtcttaagctggcgcgaggaccaacgtatcagccaggcgaagctgcttttgagcaccacccggatgcctatcgccaccgtcggtcgcaatgttggttttgacgatcaactctatttctcgcgggtatttaaaaaatgcaccggggccagcccgagcgagttccgtgccggttgtgaagaaaaagtgaatgatgtagccgtcaagttgtcataattggtaacgaatcagacaattgacggcttgacggagtagcatagggtttgcagaatccctgcttcgtccatttgacaggcacattatgcatgcaattctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatccggaaacgaatatcaacattggcaccagttacctgcaatatgtttatcagcagtttggcaataatcgtattttctcctcagcagcttataacgccggactagggcgggtgcgaacctggcttggcaacagcgccgggcgtatcgacgcagtggcatttgtcgagagtattccattctccgagacgcgcggttatgtgaagaacgtgctggcttatgacgcttactaccgctatttcatgggggataaaccgacgttgatgagcgccacggaatggggacgtcgttactgatccgcacgtttatgatatgctatcgtactctttagcgagtacaaccgggggaggcattttgcttcccccgctaacaatggcgacatattatggcccaacaatcaccctattcagcagcgatggcagaacagcgtcaccaggagtggttacgttttgtcgacctgcttaagaatgcctaccaaaacgatctccatttaccgttgttaaacctgatgctgacgccagatgagcgcgaagcgttggggactcgcgtgcgtattgtcgaagagctgttgcgcggcgaaatgagccagcgtgagttaaaaaatgaactcggcgcaggcatcgcggatcccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagggccaacgagctagcgaggacgtcgtctgctgctcgatgtcctacactggggcccgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaatttcagctgagcgccggtcgctaccattaccagttggtctggtgtcaaaaataataataaccgggcaggccatgtctgcccgtatttcgcgtaaggaaatccattatgtactatttaaa

SEQ ID NO: 68 - DNA, 7822 bp, Artificial sequence
  pEB13-lacZ NS5A/B
tttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattttataaaccgtggagcgggcaatactgagctgatgagcaatttccgttgcaccagtgcccttctgatgaagcgtcagcacgacgttcctgtccacggtacgcctgcggccaaatttgattcctttcagctttgcttcctgtcggccctcattcgtgcgctctaggatcctccggcgttcagcctgtgccacagccgacaggatggtgaccaccatttgccccatatcaccgtcggtactgatcccgtcgtcaataaaccgaaccgctacaccctgagcatcaaactcttttatcagttggatcatgtcggcggtgtcgcggccaagacggtcgagcttcttcaccagaatgacatcaccttcctccaccttcatcctcagcaaatccagcccttcccgatctgttgaactgccggatgccttgtcggtaaagatgcggttagcttttacccctgcatctttgagcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaaggggtctgacgctcagtggaacgaaaactcacgttaagggattttggtcatgagattatcaaaaaggatcttcacctagatccttttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatccggaaacgaatatcaacattggcaccagttacctgcaatatgtttatcagcagtttggcaataatcgtattttctcctcagcagcttataacgccggactagggcgggtgcgaacctggcttggcaacagcgccgggcgtatcgacgcagtggcatttgtcgagagtattccattctccgagacgcgcggttatgtgaagaacgtgctggcttatgacgcttactaccgctatttcatgggggataaaccgacgttgatgagcgccacggaatggggacgtcgttactgatccgcacgtttatgatatgctatcgtactctttagcgagtacaaccgggggaggcattttgcttcccccgctaacaatggcgacatattatggcccaacaatcaccctattcagcagcgatggcagaacagcgtcaccaggagtggttacgttttgtcgacctgcttaagaatgcctaccaaaacgatctccatttaccgttgttaaacctgatgctgacgccagatgagcgcgaagcgttggggactcgcgtgcgtattgtcgaagagctgttgcgcggcgaaatgagccagcgtgagttaaaaaatgaactcggcgcaggcatcgcggatcccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagggccaacgagctagcgaggacgtcgtctgctgctcgatgtcctacactggggcccgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaatttcagctgagcgccggtcgctaccattaccagttggtctggtgtcaaaaataataataaccgggcaggccatgtctgcccgtatttcgcgtaaggaaatccattatgtactatttaaa

SEQ ID NO: 69 - DNA, 5755 bp, Artificial sequence
  pBAD-MBP-scNS3
aagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgttttttgggctaacaggaggaattaaccatgaaaactgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcgaaagagttcctcgaaaactatctgctgactgatgaaggtctggaagcggttaataaagacaaaccgctgggtgccgtagcgctgaagtcttacgaggaagagttggcgaaagatccacgtattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcggtaccgtcctctctcgtgatcgagggtaggcctgaattcagtaccatggcgcctatcggctcagtagtaatcgtaggcagaatcatcctgtccggccgtggtggcccgatcacggcctactcccaacagacgcggggcctacttggttgcattatcactagtctcacgggccgggacaagaaccaggttgagggggaggttcaagtggtctccaccgcaacacaatctttcctggcgacctgtgtcaacggcgcgtgctggactgtctttcacggtgccggctcaaagaccttagccggcccaaaaggcccaatcacccaaatgtacaccaatgtagaccaggacctcgtcggctggccagcgccccccggggcgcgttccttaacaccgtgcacctgcggcagctcggacctttacctggtcacgagacatgctgatgtcatcccggtgcgccggcggggcgacactagggggagcttgctctcccctagacccatctcctacttgaagggctcttcgggtggtccattgctctgcccctcggggcacgttgtgggcatcttccgggctgccgtgtgcacccggggggtcgcgaaggcggtggacttcatacccgttgaatctatggaaaccactatgcggtaagcggccgcagactacaaggactaggcaagctaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagag

SEQ ID NO: 70 - protein, 12 aa, Artificial sequence
  Modified NS3 cleavage site
ASEDVVCCSMSY

SEQ ID NO: 71 - DNA, 66 bp, Artificial sequence
  primer
gaaattatcgatgagggccaacgtggccggtaatttttggcctctggggcccgtggtggttatgcc

SEQ ID NO: 72 - DNA, 21 bp, Artificial sequence
  primer
cagccgggaagggctggtctt

SEQ ID NO: 73 - DNA, 21 bp, Artificial sequence
  primer
ccggaaagctggctggagtgc

SEQ ID NO: 74 - DNA, 21 bp, Artificial sequence
  primer
ggccctcatcgataatttcac

SEQ ID NO: 75 - DNA, 41 bp, Artificial sequence
  primer
agctagcgaggacgtcgtctgctgctcgatgtcctacactg

SEQ ID NO: 76 - DNA, 41 bp, Artificial sequence
  primer
tgtaggacatcgagcagcagacgacgtcctcgctagctcgt

SEQ ID NO: 77 - DNA, 32 bp, Artificial sequence
  primer
tcagtaccatggcgcctatcggctcagtagta

SEQ ID NO: 78 - DNA, 38 bp, Artificial sequence
  primer
gggaaagcggccgcttaccgcatagtggtttccataga

SEQ ID NO: 79 - DNA, 29 bp, Artificial sequence
  primer
ccctaatcatgaaaactgaagaaggtaaa

SEQ ID NO: 80 - DNA, 25 bp, Artificial sequence
  primer
aattgcatgcctgcaggcggccgcg

SEQ ID NO: 81 - DNA, 25 bp, Artificial sequence
  primer
aattcgcggccgcctgcaggcatgc

SEQ ID NO: 82 - DNA, 7804 bp, Artificial sequence
  pEB13-Sfi 279-280
tttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattttataaaccgtggagcgggcaatactgagctgatgagcaatttccgttgcaccagtgcccttctgatgaagcgtcagcacgacgttcctgtccacggtacgcctgcggccaaatttgattcctttcagctttgcttcctgtcggccctcattcgtgcgctctaggatcctccggcgttcagcctgtgccacagccgacaggatggtgaccaccatttgccccatatcaccgtcggtactgatcccgtcgtcaataaaccgaaccgctacaccctgagcatcaaactcttttatcagttggatcatgtcggcggtgtcgcggccaagacggtcgagcttcttcaccagaatgacatcaccttcctccaccttcatcctcagcaaatccagcccttcccgatctgttgaactgccggatgccttgtcggtaaagatgcggttagcttttacccctgcatctttgagcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaaggggtctgacgctcagtggaacgaaaactcacgttaagggattttggtcatgagattatcaaaaaggatcttcacctagatccttttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatccggaaacgaatatcaacattggcaccagttacctgcaatatgtttatcagcagtttggcaataatcgtattttctcctcagcagcttataacgccggactagggcgggtgcgaacctggcttggcaacagcgccgggcgtatcgacgcagtggcatttgtcgagagtattccattctccgagacgcgcggttatgtgaagaacgtgctggcttatgacgcttactaccgctatttcatgggggataaaccgacgttgatgagcgccacggaatggggacgtcgttactgatccgcacgtttatgatatgctatcgtactctttagcgagtacaaccgggggaggcattttgcttcccccgctaacaatggcgacatattatggcccaacaatcaccctattcagcagcgatggcagaacagcgtcaccaggagtggttacgttttgtcgacctgcttaagaatgcctaccaaaacgatctccatttaccgttgttaaacctgatgctgacgccagatgagcgcgaagcgttggggactcgcgtgcgtattgtcgaagagctgttgcgcggcgaaatgagccagcgtgagttaaaaaatgaactcggcgcaggcatcgcggatcccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagggccaacgtggccggtaatttttggcctctggggcccgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaatttcagctgagcgccggtcgctaccattaccagttggtctggtgtcaaaaataataataaccgggcaggccatgtctgcccgtatttcgcgtaaggaaatccattatgtactatttaaa

SEQ ID NO: 83 - protein, 14 aa, Artificial sequence
  scFv-171 binder
CCGGGWANNNPGNC

SEQ ID NO: 84 - protein, 14 aa, Artificial sequence
  scFv-171 binder
CCQTLQDHERKPEC

SEQ ID NO: 85 - protein, 14 aa, Artificial sequence
  scFv-171 binder
CSKMEKCTQNKLEC

SEQ ID NO: 86 - protein, 14 aa, Artificial sequence
  scFv-171 binder
CRHYRSYNYSRCSC

SEQ ID NO: 87 - protein, 14 aa, Artificial sequence
  scFv-171 binder
CDCKDSTKKGATSC

SEQ ID NO: 88 - protein, 14 aa, Artificial sequence
  scFv-171 binder
CGPGETCRKGIFHC

SEQ ID NO: 89 - protein, 11 aa, Artificial sequence
  scFv-171 binder
CSDCANADSLC

SEQ ID NO: 90 - protein, 14 aa, Artificial sequence
  scFv-171 binder
CITVQEMCDERDNC

SEQ ID NO: 91 - protein, 13 aa, Artificial sequence
  scFv-171 binder
CCRKGTQSTHKAC

SEQ ID NO: 92 - protein, 14 aa, Artificial sequence
  scFv-171 binder
CAYHCATTRTFGAC

SEQ ID NO: 93 - protein, 14 aa, Artificial sequence
  scFv-171 binder
CAQQFEITNCRDQC

SEQ ID NO: 94 - protein, 14 aa, Artificial sequence
  scFv-171 binder
CTVSGSVCRYFWSC

SEQ ID NO: 95 - protein, 14 aa, Artificial sequence
  scFv-171 binder
CDRNTDSGRCGRVC

SEQ ID NO: 96 - protein, 14 aa, Artificial sequence
  scFv-171 binder
CRCIRPNNSPEGEC

SEQ ID NO: 97 - protein, 14 aa, Artificial sequence
  scFv-171 binder
CERGDLECGDTINC

SEQ ID NO: 98 - protein, 14 aa, Artificial sequence
  scFv-171 binder
CPMRSHSCNRVVHC

SEQ ID NO: 99 - protein, 14 aa, Artificial sequence
  scFv-171 binder
CTQIPEARACPWKC

SEQ ID NO: 100 - protein, 14 aa, Artificial sequence
  scFv-35 binder
CILSISKCGSGVNC

SEQ ID NO: 101 - protein, 6 aa, Artificial sequence
  scFv-35 binder
CHPALC

SEQ ID NO: 102 - protein, 14 aa, Artificial sequence
  scFv-35 binder
CNCRSWQEMWDRAC

SEQ ID NO: 103 - protein, 14 aa, Artificial sequence
  scFv-35 binder
CAYPEGLSAGSEPW

SEQ ID NO: 104 - protein, 14 aa, Artificial sequence
  scFv-35 binder
CFYGVCIGTVDNRC

SEQ ID NO: 105 - protein, 7 aa, Artificial sequence
  scFv-35 binder
LRGVGGQ

SEQ ID NO: 106 - protein, 7 aa, Artificial sequence
  scFv-35 binder
CVASYLI

SEQ ID NO: 107 - protein, 14 aa, Artificial sequence
  scFv-35 binder
CVNPCSRSVETAIC

SEQ ID NO: 108 - protein, 14 aa, Artificial sequence
  scFv-35 binder
CGTRSSLCELQSRC

SEQ ID NO: 109 - protein, 14 aa, Artificial sequence
  scFv-35 binder
CRATTTPLTITPKC

SEQ ID NO: 110 - protein, 14 aa, Artificial sequence
  epitope mimetic peptide
GARSLTPCTCGSSD

SEQ ID NO: 111 - protein, 21 aa, Artificial sequence
  control peptide
TLDPRSFLLRNPNDKYEPFWK

SEQ ID NO: 112 - protein, 8 aa, Artificial sequence
  control peptide
ISEVNLDA

SEQ ID NO: 113 - protein, 56 aa, Artificial sequence
  dAb inhibitor
GKATMTADTSSNTAYLQLSSLTSEDTAVYYCNAWGGSSDYYAMDYWGQGTSVTVSS

SEQ ID NO: 114 - protein, 446 aa, Artificial sequence
  MBP-dAb inhibitor
MAMKTEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTTVDEALKDAQTNSSSVPSSLVIEGRPEFSTMGKATMTADTSSNTAYLQLSSLTSEDTAVYYCNAWGGSSDYYAMDYWGQGTSVTVSS

SEQ ID NO: 115 - DNA, 1905 bp, Artificial sequence
  MBP-1(Fv)
atgaaaactgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcgaaagagttcctcgaaaactatctgctgactgatgaaggtctggaagcggttaataaagacaaaccgctgggtgccgtagcgctgaagtcttacgaggaagagttggcgaaagatccacgtattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcggtaccgtcctctctcgtgatcgagggtaggcctgaattcagtaccatggccgaggttcagctgcagcaatctggacctgagctggtgaagcctggggcttcagtgaagatgtcctgtaaggcttctggatacacattcactgactactacataaagtgggtgaagcagagtcatggaaagagccttgagtggattggagtgattaatcctggaagtggtggtactaactacaatgagaagttcaagggcaaggcaacactgactgcagacaaatcctccagcactgcctacatgcagctcagcagcctgacttctgagaactctgcagtctatttctgtgcaagaagggctgggacggggtactactttgactactggggccaaggcaccacagtcacagtctcctcgggaggtggtggatccggcggtggcggttctggtggaggtggatctgatgttttgatgacccaaactccactctccctgcctgtcagtcttggagatcaagcctccatctcttgcagatctagtcagagcattgtacatagtaatggaaacacctatttagaatggtacctgcagaaaccaggccagtctccaaagctcctgatctacaaagtttccaaccgattttctggggtcccagacaggttcagtggcagtggatcagggacagatttcacactgaaaatcagcagagtggaggctgaggatctgggagtttattactgctttcaaggttcacatgttccgctcacgttcggtgctgggacaaaactggagatcaaacgg

SEQ ID NO: 116 - DNA, 1899 bp, Artificial sequence
  MBP-10(Fv)
atgaaaactgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcgaaagagttcctcgaaaactatctgctgactgatgaaggtctggaagcggttaataaagacaaaccgctgggtgccgtagcgctgaagtcttacgaggaagagttggcgaaagatccacgtattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcggtaccgtcctctctcgtgatcgagggtaggcctgaattcagtaccatggcccaggtccagctgcagcagtctggagctgagctgatgaagcctggggcctcagtgaagatatcctgcaaggctactggctacacattcagtaactactggatagagtgggtaaagcagaggcctggacatggccttgagtggattggagggattttacctggaagtggtagtactaactacaatgagaagttcaagggcaaggccacattcactgcagatacatcctccaacacagcctacctgcagctcagcagcctgacatctgaggacactgccgtctattactgtaatgcggatggccctccggggtttgactactggggccaaggcaccacgctcacagtctcctcgggaggtggtggatccggcggtggcggttctggtggaggtggatctgatgttttgatgacccaaactccactcactttgtcggttaccattggacaaccagcctccatctcttgcaagtcaagtcagagcctcttagatagtgatggaaagacatatttgaattggttgttacagaggccaggccagtctccaaagcgcctaatctatctggtgtctaaactggactctggagtccctgacaggttcactggcagtggatcagggacagatttcacactcaagatcagcagagtggaggctgaggatctgggagtttattactgctttcaaggttcacatgttccattcacgttcggctccggcacaaaactggaaataaaacgg

SEQ ID NO: 117 - DNA, 1890 bp, Artificial sequence
  MBP-11(Fv)
atgaaaactgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcgaaagagttcctcgaaaactatctgctgactgatgaaggtctggaagcggttaataaagacaaaccgctgggtgccgtagcgctgaagtcttacgaggaagagttggcgaaagatccacgtattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcggtaccgtcctctctcgtgatcgagggtaggcctgaattcagtaccatggccgaggttcagctgcagcagtctggggcagagcttgtgaagccaggggcctcagtcaagttgtcctgcacagcttctggcttcaacattaaagactactatatgcactgggtgaagcagaggcctggacagggtcttgagtacattgggcggatttatcctggaaatggacaaactgactacaatgggaagttcaagggcaaggccacgctgactgcagacaagtcctccagcacagcctacatgcagctcagcagcctgacctctgtggactctgcggtctatttctgtgcaagaggtctgtttgcttactggggccaagggactctggtcaccgtctcttcgggaggtggtggatccggcggtggcggttctggtggaggtggatctgatgttgtgatgacccagactccactctccctgcctgtcagctttggagatcaagtttctatctcttgcaggtctagtcagagtcttgcaaacagttatgggaacacctatttgtcttggtgcctgcacaagcctggccagtctccacagctcctcatctatgggatttccaacagattttctggggtgccagacaggttcagtggcagtggttcagggacagatttcacactcaagatcagcacaataaagcctgaggacttgggaatgtattactgcttacaaggtacacatcagccgctcacgttcggtgctgggaccaagctggagatcaaacgg

SEQ ID NO: 118 - DNA, 1893 bp, Artificial sequence
  MBP-12(Fv)
atgaaaactgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcgaaagagttcctcgaaaactatctgctgactgatgaaggtctggaagcggttaataaagacaaaccgctgggtgccgtagcgctgaagtcttacgaggaagagttggcgaaagatccacgtattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcggtaccgtcctctctcgtgatcgagggtaggcctgaattcagtaccatggccgaggtcaagttgcaacagtctggaactgaactggcaagacctggggcttcagtgaagttgtcctgcaaggcttctggctacacctttactagctactggatgcagtggatgaaacagaggcctggacagggtctggaatggattggggctatttatcctggagatggtgatactaggtacactcagaagttcaagggcaaggccacattgactgcagataaatcctccagcacagcctacatgcaactcagcagcttggcatctgaggactctgcggtctattactgtgcaagatcgagggctatggacgactggggtcaaggaacctcgctcacagtctcctcgggaggtggtggatccggcggtggcggttctggtggaggtggatctgatgttttgatgacccaaactccactctccctgcctgtcagtcttggagatcaagcctccatctcttgcagatctagtcagagcattgtacatagtaatggaaacacctatttagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcacatgttcctcggacgttcggtggagggaccaaactggagataaaacgg

SEQ ID NO: 119 - DNA, 1908 bp, Artificial sequence
  MBP-31(Fv)
atgaaaactgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcgaaagagttcctcgaaaactatctgctgactgatgaaggtctggaagcggttaataaagacaaaccgctgggtgccgtagcgctgaagtcttacgaggaagagttggcgaaagatccacgtattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcggtaccgtcctctctcgtgatcgagggtaggcctgaattcagtaccatggcccaggtccacttgcaccagtctggacctgagctggtgaagcctggggcttcagtgaagctgtcctgcaagacttctggcttcaccttcagcagtaccttttttagttggttgaagcagaagcctggacagagtcttgagtggattgcatggatttatgctggatctggtggttctagctataatcagaagttcacaggcaaggcccaactgactgtagacacatcctccagcacagcctacatacaattcagcagcctgacaactgaggactctgccatctattactgtgcaagacaaaacctcgattacgactatgctatggactactggggtcaaggaacctctgtcacagtctcctcgggaggtggtggatccggcggtggcggttctggtggaggtggatctgatgttttgatgacccaaactccactctccctgcctgtcagtcttggagatcaagcctccatctcttgcagatctagtcagagccttgtacacagtaatggaaacacctatttacattggtacctgcagaagccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaagtacacatgttccgtacacgttcggagggggcaccaagctggaaatcaaacgg

SEQ ID NO: 120 - DNA, 1902 bp, Artificial sequence
  MBP-35(Fv)
atgaaaactgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcgaaagagttcctcgaaaactatctgctgactgatgaaggtctggaagcggttaataaagacaaaccgctgggtgccgtagcgctgaagtcttacgaggaagagttggcgaaagatccacgtattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcggtaccgtcctctctcgtgatcgagggtaggcctgaattcagtaccatggccgaggtccagctgcagcaatctggagcagagcttgtgaggtcaggggcctcagtcaagttgtcctgcacagcttctggcttcaacattaaagactactatatgcactgggtgaagcagaggcctgaacagggcctggagtggattggatggattgatcctgagaatggtgatactgaatacactcagaagttcaagggcaaggccacattgactgcagataaatcccccagcacagcctacatgcaactgagcagcctgacatctgaggactctgcagtctattactgtgcaagaattactacggattactactttgactactggggccaaggcaccacgctcaccgtctcctcgggaggtggtggatccggcggtggcggttctggtggaggtggatctgatgttgtgatgacccaaactccactctccctgcctgtcagtcttggagatcaagcctccatctcttgcagatctagtcagagccttgtacatagtaatggaaacacctatttagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaagtacacatgttcctctcacgttcggtgctgggaccaaactggagatcaaacgg

SEQ ID NO: 121 - DNA, 1902 bp, Artificial sequence
  MBP-148(Fv)
atgaaaactgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcgaaagagttcctcgaaaactatctgctgactgatgaaggtctggaagcggttaataaagacaaaccgctgggtgccgtagcgctgaagtcttacgaggaagagttggcgaaagatccacgtattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcggtaccgtcctctctcgtgatcgagggtaggcctgaattcagtaccatggccgagatccagctgcagcagtctggggcagagcttgtgaagccaggggcctcagtcaagttgtcctgcacagcttctggcttcaacattaaagacacctttgtgcactgggtgaaacagaggcctgaacagggcctggagtggattggaaggattgatcctgcgaatggtaatactaaatatgacccgaagttccagggcaaggccactataacagcagccacatcctccaacacagcctacctgcagctcagcagcctgacatctgaggacactgccgtctattattgtgctacagggctagattactatgctatggactactggggtcaaggaacctcagtcacagtctcctcgggaggtggtggatccggcggtggcggttctggtggaggtggatctgatgttgtgatgacccagactcccctctccccgcctgtcagtcttggagatcaagcctccatctcttgcagatctagtcagagccttgtacatagtaatggaaacacctatttacattggtacctgcagaagccaggccagtctccaaagctcctgatctacaaagtttccaaccgcttttctggggtcccagacaggttcagtggcagtggatcagggacagatttcacactcaagatcaccagagtggaggctgaggatctgggactttatttctgctctcaaagtacacatgttccgtggacgttcggtggaggcacaaaactggaaatcaaacgg

SEQ ID NO: 122 - DNA, 1911 bp, Artificial sequence
  MBP-152(Fv)
atgaaaactgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcgaaagagttcctcgaaaactatctgctgactgatgaaggtctggaagcggttaataaagacaaaccgctgggtgccgtagcgctgaagtcttacgaggaagagttggcgaaagatccacgtattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcggtaccgtcctctctcgtgatcgagggtaggcctgaattcagtaccatggcccaggttcaggtgcaaccatctgggacagagcttgtgaagccaggggcctcagtcaagttgtcctgcgcaacttctggcttcaacattaaagacacctatatgcagtgggtgaagcagaggcctgaacagggcctggagtggattggatggattgatcctgagaatggtgatactgaatctgccccgaagttccagggcaaggccactatgactgcagacacatcctccaacacagcctacctgcagctcagcagcctgacatctgaggactctgccgtctatttctgtgcaagagaaagggattactacggctacgaggggtttgcttactggggccaagggactctggttactgtctcttcgggaggtggtggatccggcggtggcggttctggtggaggtggatctgatgttgtgatgacccaaactccactctccctgcctgtcagtcttggagatcaagcctccatctcttgcagatctagtcagagccttgtacatagtaatggaaacacctatttagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaagtacacatgttcctctcacgttcggtgctgggaccaaactggagatcaaacgg

SEQ ID NO: 123 - DNA, 1884 bp, Artificial sequence
  MBP-161(Fv)
atgaaaactgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcgaaagagttcctcgaaaactatctgctgactgatgaaggtctggaagcggttaataaagacaaaccgctgggtgccgtagcgctgaagtcttacgaggaagagttggcgaaagatccacgtattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcggtaccgtcctctctcgtgatcgagggtaggcctgaattcagtaccatggcccaggtccagctgcaacagcctggggcagagcttgtgaagccaggggcctcagtcaagttgtcctgcacagcttctggcttcaacattaaagacacctatatgcactgggtgaagcagaggcctgaacagggcctggagtggattggaaggattgatcctgcgaatggtaatactaaatatgacccgaagttccagggcaaggccactataacagcagacaaatcctccagcacagcctacatgcaactcagcagcctgacatctgaggactctgcggtctattactgtacaagaatctggctaccctggggccgagggactctggttactgtctcttcgggaggtggtggatccggcggtggcggttctggtggaggtggatctgatgttttgatgacccaaactccactctccctgcctgacagtcttggagatcaagcctccatctcttgcagatctagtcagagcattgtacatagtaatggaaacacctatttagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaagtacacatgttctcacgttcggtgctgggaccaagctggagctgaaacgg

SEQ ID NO: 124 - DNA, 1896 bp, Artificial sequence
  MBP-162(Fv)
atgaaaactgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcgaaagagttcctcgaaaactatctgctgactgatgaaggtctggaagcggttaataaagacaaaccgctgggtgccgtagcgctgaagtcttacgaggaagagttggcgaaagatccacgtattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcggtaccgtcctctctcgtgatcgagggtaggcctgaattcagtaccatggccgaggtccaggtgcaacagtctggggcagagcctgtgaagtcaggggcctcagtcaagttgtcctgcacagctttcggcttcaacattaaagactactatatgcactgggtgaagcagaggcctgaacagggcctggagtggattggatggattgatcctgcgaatggtaatactaaatatgacccgaagttccagggcaaggccactataacagcagacacatcctccaacacagcctacctgcagctcagcagcctgacatctgaggacactgccgtctattactgtgctagatgggactggtacttcgatgtctggggcgcagggaccacagtcacagtctcctcgggaggtggtggatccggcggtggcggttctggtggaggtggatctgatgttttgatgacccaaactccactctccctgcctgtcagtcttggagatcaagcctccatctcttgcagatctagtcagagcattgtacatagtaatggaaacacctatttagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcacatgttccgtggacgttcggtggagggaccaagctggaactgaaacgg

SEQ ID NO: 125 - DNA, 1811 bp, Artificial sequence
  MBP-171(Fv)
ctggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcgaaagagttcctcgaaaactatctgctgactgatgaaggtctggaagcggttaataaagacaaaccgctgggtgccgtagcgctgaagtcttacgaggaagagttggcgaaagatccacgtattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcggtaccgtcctctctcgtgatcgagggtaggcctgaattcagtaccatggccgaggtccagctgcagcagtctggggcagagctggtgaggtcaggggcctcagtcaagttgtcctgcacagcttctggcttcaacattaaagactactatatgcactgggtgaagcagaggcctgaacagggcctggagtggattggatggattgatcctgagaatggtgatactgaatatgccccgaagttccagggcaaggccactatgactgcagacacatcctccaacacagcctacctgcagctcagcagcctgacatctgaggacactgccgtctattactgtaatgcatggggcggtagtagtgattactatggtatggactactggggtcaaggaacctcggtcaccgtctcctcgggaggtggtggatccggcggtggcggttctggtggaggtggatctgatgttgtgatgacccaaactccactctccctgcctgtcagtcttggagatcaagcctccatctcttgcagatctagtcagagccttgtacacagtaatggaaacacctatttacattggtacctgcagaagccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcacatgttccgtggacgttcggtggaggcaccaaactggagatcaaacgggcggccgcaggcggtctgaacgacatcttcgaagctcagaaaatcgaatggcacgaataa

(19) United States
(12) Patent Application Publication
(10) Pub. No.: US 2008/0089862 A1
(43) Pub. Date: Apr. 17, 2008

(54) PROTEASE INHIBITORS AND METHOD OF SCREENING THEREOF

(76) Inventors: Itai BENHAR, Rehovot (IL); Ran Tur-Kaspa, Mevaseret Zion (IL); Romy Zemel, Tel-Aviv (IL); Meital Gal-Tanamy, Rosh Ha'ayin (IL); Alla Trachtenherz, Moshav Herut (IL); Orly Pupko, Herzliya (IL); Jonathan M. Gershoni, Herzliya (IL)

Correspondence Address: WINSTON & STRAWN LLP;PATENT DEPARTMENT, 1700 K STREET, N.W., WASHINGTON, DC, 20006, US

(21) Appl. No.: 11/839,301

(22) Filed: Aug. 15, 2007

Related U.S. Application Data
(63) Continuation of application No. PCT/IL06/00245, filed on Feb. 22, 2006.
(60) Provisional application No. 60/654,446, filed on Feb. 22, 2005.

(51) Int. Cl. A61K 31/70; A61K 38/00; A61P 43/00; C07H 21/04; C07K 16/00; C12N 15/00; C12N 5/00; C12Q 1/37

(52) U.S. Cl. 424/93.1; 435/23; 435/320.1; 435/325; 514/44; 530/350; 536/22.1; 536/23.1

(57) ABSTRACT

The present invention provides novel recombinant protein and peptide inhibitors of NS3 serine protease of the hepatitis C virus (HCV). The invention discloses analogs, fragments and derivatives of the identified inhibitors, nucleic acids encoding same, and methods of use thereof for the treatment of HCV infection. The invention further provides novel constructs and methods for the screening of protease inhibitors in vivo, using a recombinant engineered reporter protein that is cleavable by a protease, co-expressed with the recombinant protease in bacteria.

### CROSS-REFERENCE TO RELATED APPLICATIONS

This application is a continuation of International application PCT/IL2006/000245 filed Feb. 22, 2006 and claims the benefit of application No. 60/654,446 filed Feb. 22, 2005. The entire content of each prior application is expressly incorporated herein by reference thereto.

### BACKGROUND

The present invention provides novel NS 3 serine protease inhibitors, analogs, fragments and derivatives thereof, nucleic acids encoding same, and methods of use thereof for the treatment of Hepatitis C virus (HCV) infection. The present invention further provides a reporter gene system and method of use thereof for screening of protease inhibitors in a host cell.

Hepatitis C virus: Hepatitis C virus (HCV) is an RNA virus that causes hepatitis, cirrhosis, liver failure and hepatocellular carcinoma (HCC). Development of HCC may occur in up to 10% of HCV-infected individuals. Globally, the seroprevalence of HCV is over 170 million. This implies that over 10 million individuals are at risk for HCV-associated HCC. The magnitude of this potential cancer burden presents an impetus to understand the transforming mechanism(s) of this virus. Currently, the viral-encoded NS3 is one of the viral candidate oncoproteins.

HCV, a member of the Flaviviridiae family, is a small enveloped virus with a single-stranded, positive-sense RNA genome packed within a nucleocapsid. The 9.6 kb RNA genome is organized to contain a single, large translational open-reading frame that spans most of its length. This encodes a large polyprotein precursor of 3010-3033 amino acids. Four structural and at least six nonstructural (NS) proteins are initially generated by co-translational cleavage of the polyprotein by both cellular and virally-encoded proteases. Most subsequent proteolytic processing events are directed by the virally-encoded NS3 serine protease that requires the adjacent NS4A cofactor for efficient cleavage activity.

NS3 Protease: The HCV trypsin-like serine protease activity resides in the amino-terminal third of the NS3 protein. The mature form of the NS3 is a bifunctional protein having also NTPase and helicase activities located within its carboxyl-terminal domain. NS3 forms a noncovalent complex with NS4A and as such directs proteolytic cleavages at the NS3/4A, NS4A/4B, NS4B/5A, and NS5A/5B junctions and is thus essential for replication of the virus. The crystal structure of the NS3 serine protease has been elucidated and much is known about its structure-function relations. Some enzymatic and structural features make NS3 unique among the serine proteases family. The serine protease domain of NS3 requires unusually long substrates (P6-P4′) for effective cleavage and possesses a solvent-accessible structural zinc-binding site. In addition, expression of NS3 has been found to interfere with signal transduction pathways, promote cell proliferation and cause cell transformation. The inventors of the present invention have recently reported that NS3-mediated cell transformation is dependent of its being catalytically active (Zemel et al., 2001).

The quest for NS3 inhibitors: The development of new anti-HCV protease inhibitors is dependent on effective schemes for catalysis and inhibition assays in vitro and in vivo. High throughput screenings using in vitro assays with purified protease and synthetic substrates and structure-based drug design peptide are currently being used in the search for inhibitors. Because the substrate-binding pocket of NS3 is shallow, the binding of small molecule inhibitors is quite inefficient. This explains why previous searches for a good lead inhibitor starting from known protease inhibitors were unsuccessful. Biochemical studies with known protease inhibitors had revealed that the NS3 is inhibited by unusually high concentrations of chymotrypsin-like serine protease inhibitors or by general serine protease inhibitors (Kwong et al., 1998, U.S. Pat. Application No. 20030083467). The observation that the addition of high conventional serine protease inhibitors concentrations resulted in either no inhibition or very weak inhibition of the NS3 protease suggests that the active site of the protease is different from that of cellular serine proteases and that specific non-toxic inhibitors of NS 3 could be developed.

Because of the difficulties of de novo design of non-toxic small molecule inhibitors to bind to the NS3 protease-binding pocket, many groups have turned to high throughput screening of chemical and natural product libraries to search for novel lead molecules. However, in most cases the tested compounds also inhibited human serine proteases such as chymotrypsin and elastase making them inappropriate candidates for clinical use (Reviewed in Kwong et al., 1998). Some groups have turned to screening of libraries of ‘minimized’ antibody-like proteins and single-chain antibodies for NS3 protease inhibitors (Martin et al., 1999).

Bioassays for NS3 Protease Activity and HCV Anti-Viral Assays

The development of secondary in vivo bioassays to test whether an inhibitor identified through an in vitro assay can fulfill its function in a cellular environment is a critical step in the drug development process. However, several obstacles have hindered efforts at antiviral drug discovery. Foremost has been the absence of fully permissive cell cultures allowing efficient in vitro propagation of the virus. This, coupled with the lack of a readily available animal model of chronic hepatitis C, has rendered it difficult to identify and validate lead compounds with potentially useful antiviral activities. Nonetheless, the recent development of subgenomic HCV RNA replicons has provided robust in vitro systems for characterizing the replication of the viral RNA in cultured cells. Still, the testing of candidate anti-viral molecules has been limited by the lack of a robust system for growing HCV in cultured cells (reviewed in Lindenbach and Rice, 2005). A cell-based system has been described in which NS3 protease is required for modulation of a reporter gene (Hirowatari et al., 1993). Other in vivo assays included the development of chimeric sindbis and polio viruses (Hahm et al., 1996) whose viral replication is dependent on NS3 protease activity. Such systems were designed as secondary to primary in vitro screenings to allow investigators to study the potential of NS3 protease inhibitors. However, none of them is of a high-throughput nature.

Genetic Screenings for Protease Inhibitors

Genetic screenings for ˆproteases, and protease inhibitors in general (Dautin et al., 2000; Martinez et al., 2000) and for NS3 inhibitors in particular (Martinez and Clotet 2003) have been described but came short of providing the desired protease inhibitors. With regard to β-galactosidase engineered with protease cleavage sites, Baum et al., (Baum et al., 1990) inserted the HIV protease site into several positions of the lacZ gene and tested the resultant engineered derivatives for β-galactosidase activity and for the ability to be cleaved by the HIV protease. Their strategy involved cloning into unique restriction sites that are scattered along the lacZ gene and did not involve prior evaluation of the permissiveness of these sites for peptide insertion. Indeed, most of the derivatives they created lost most of the β-galactosidase enzymatic activity (still, however, growing as blue colonies on X-gal indicative plates). The derivative they chose to further evaluate for cleavage had the site inserted after residue 80. This derivative could be cleaved both in vitro and in vivo (co-expressed with the protease in E. coli) by recombinant HIV protease. The cleavage products could be observed by an immunoblot using anti β-galactosidase sera. More recently, Cheng et al. (Cheng et al., 2004) co-expressed recombinant HIV protease and β-galactosidase with the cleavage site inserted after residue 131. The cleavage products could be detected by an immunoblot as in the previous study. This group applied bacteria that express the HIV protease and the engineered β-galactosidase to screen a library of chemical compounds based on a sulfonamide isostere core and identified candidate inhibitors of the HIV protease.

NS3 is essential for HCV viral replication, and thus it has been an attractive target for drug discovery for the last few years. Several patents disclose NS3 inhibitors (see, for example, U.S. Pat. Nos. 6,608,027, 6,774,212 and 6,767,991).

Inhibitors of the HCV NS3 protease have been described in international applications WO 02/18369, WO 00/09543 (Boehringer Ingelheim), WO 03/064456 (Boehringer Ingelheim), WO 03/064416 (Boehringer Ingelheim), WO 02/060926 (Bristol-Myers Squibb), WO 03/053349 (Bristol-Myers Squibb), WO 03/099316 (Bristol-Myers Squibb), WO 03/099274 (Bristol-Myers Squibb), WO 2004/032827 (Bristol-Myers Squibb), and WO 2004/043339 (Bristol-Myers Squibb).

However, to date there are no serine protease inhibitors available as FDA-approved anti-HCV agents (reviewed in De Francesco and Migliaccio, 2005).

There are currently few effective treatments for HCV. The most established treatment for HCV patients includes administration of recombinant interferon alpha. However, interferons have significant side effects and induce long-term remission in only a fraction (about 25%) of cases. Other agents used to treat chronic hepatitis C include the nucleoside analog ribovirin and ursodeoxycholic acid; however, neither has been shown to be very effective. Moreover, the prospects for effective anti-HCV vaccines remain uncertain (reviewed in De Francesco and Migliaccio, 2005). Thus, there remains a need for more effective anti-HCV therapies. An efficient, high-throughput method of screening for protease inhibitors, and the identification of protease inhibitors, particularly NS3 protease inhibitors, would be highly advantageous.

### SUMMARY OF THE INVENTION

The present invention provides novel NS3 serine protease inhibitors, analogs, fragments and derivatives thereof, nucleic acids encoding same, and methods of use thereof for the treatment of hepatitis C virus (HCV) infection. The invention further provides high throughput methods of screening for protease inhibitors in vivo, using a recombinant engineered reporter protein that is cleavable by a protease, co-expressed with the recombinant protease in bacteria.

The invention is based in part on the generation of a novel genetic screening for inhibitors of NS3 catalysis, comprising a recombinant engineered reporter protein that is cleavable by a protease, co-expressed with the protease in a host cell. The reporter protein, a recombinant β-galactosidase comprising an NS3 cleavage site in a permissive site, was surprisingly discovered to undergo proteolytic degradation upon its cleavage by the protease. The resulting genetic screening thus allows a highly sensitive, high throughput screening method, which is advantageous to other screening methods, as it is not subject to product inhibition due to accumulation of cleavage products (to that affect, NS3 itself is a protease subject to product inhibition).

The invention is further based, in part, on the discovery of novel NS3 inhibitors isolated by the genetic screening of the invention. Surprisingly, epitope mapping of certain scFv antibody inhibitors isolated according to the invention revealed a novel NS3 epitope overlapping with the NS3 zinc-binding site. This surprising discovery further confirms the uniqueness of the isolated inhibitors, as well as the ability of the genetic screening of the invention to identify such novel inhibitors that could not be identified by other screening methods.

In one aspect, the present invention provides novel NS3 inhibitors. In one embodiment, the inhibitor is a single-chain antibody (scFv) having an amino acid sequence as set forth in any one of SEQ ID NOS: 1-11. In another embodiment, the inhibitor is a single antibody domain protein (dAb) derived from the isolated scFv inhibitors of the invention. In a further embodiment, the inhibitor is a dAb having an amino acid sequence as set forth in any one of SEQ ID NOS: 12-14 and 113.

In another embodiment, the scFv or dAb is fused to a stabilizing protein. In a preferred embodiment, the stabilizing protein is E. coli maltose binding protein (MBP). In another embodiment, the scFv or dAb is fused to the C terminus of MBP. According to another embodiment, the inhibitor is a scFv fused to the C terminus of MBP, having an amino acid sequence as set forth in any one of SEQ ID NOS: 15-25. In another embodiment, the inhibitor is a dAb fused to the C terminus of MBP, having an amino acid sequence as set forth in any one of SEQ ID NOS:26-28 and 114. Other embodiments include fragments, homologs, analogs and derivatives thereof.

In another embodiment, the inhibitor is a peptide having an amino acid sequence as set forth in any one of SEQ ID NOS:29-35. In another embodiment, the inhibitor is a peptide aptamer, comprising a peptide inhibitor of the invention fused to a stabilizing protein. In a preferred embodiment, the stabilizing protein is E coli maltose binding protein (MBP). In one preferred embodiment, the peptide is fused to the C terminus of MBP. In another preferred embodiment, the peptide is fused at internal permissive positions of MBP. In another preferred embodiment, the peptide is fused at the internal permissive position following position 133 of MBP. According to another embodiment, the inhibitor is a free peptide derived from a peptide aptamer. In another embodiment, the peptide aptamer has an amino acid sequence as set forth in any one of SEQ ID NOS:36-49. Other embodiments include fragments, homologs, analogs and derivatives thereof.

In another embodiment, the invention comprises nucleic acids encoding the NS3 inhibitors of the invention. In a currently preferred embodiment, the nucleic acids encoding the NS3 inhibitors are as set forth in any one of SEQ ID NOS:115-125.

According to additional aspects the invention provides expression vectors and host cells comprising nucleic acid sequences encoding the NS3 inhibitors of the invention.

In another aspect, the invention provides pharmaceutical compositions comprising the NS3 inhibitors of the invention and a pharmaceutically acceptable carrier or excipient. In another aspect, the invention provides pharmaceutical compositions comprising nucleic acid sequences encoding the NS3 inhibitors of the invention, expression vectors or host cells comprising same.

In other aspects, the invention provides methods of: 1) treating HCV infection; 2) treating or preventing hepatitis; 3) preventing liver failure; 4) preventing chirrhosis; or 5) preventing hepatocellular carcinoma (HCC), in a subject in need thereof, comprising administering to the subject a therapeutically effective amount of a pharmaceutical composition comprising an NS3 inhibitor of the invention.

According to other aspects, the invention provides methods of: 1) treating HCV infection; 2) treating or preventing hepatitis; 3) preventing liver failure; 4) preventing chirrhosis; or 5) preventing hepatocellular carcinoma (HCC), in a subject in need thereof, comprising administering to the subject a therapeutically effective amount of a pharmaceutical composition comprising a recombinant construct comprising an isolated nucleic acid sequence encoding an NS3 inhibitor of the invention operably linked to one or more transcription control elements.

In further aspects, the invention provides methods of: 1) treating HCV infection; 2) treating or preventing hepatitis; 3) preventing liver failure; 4) preventing chirrhosis; or 5) preventing HCC, in a subject in need thereof, comprising: a) obtaining cells from the subject; b) contacting the cells a vivo with a therapeutically effective amount of a pharmaceutical composition comprising a recombinant construct comprising an isolated nucleic acid sequence encoding NS3 inhibitor of the invention operably linked to one or more transcription control elements; and c) re-introducing said cells to said subject.

In other aspects, the invention provides method of preventing HCV infection in a liver transplant, comprising: a) treating a liver transplant before transplantation ex vivo with a therapeutically effective amount of a pharmaceutical composition comprising a recombinant construct comprising an isolated nucleic acid sequence encoding NS3 inhibitor of the invention operably linked to one or more transcription control elements; and b) transplanting the liver transplant to a subject in need thereof, thereby generating an HCV-immune liver transplant.

According to additional aspects, the invention provides novel recombinant reporter gene constructs and uses thereof for the screening and isolation of protease inhibitors, as described hereinbelow.

In one embodiment, the invention provides an isolated polynucleotide comprising a nucleic acid sequence encoding a recombinant reporter protein, wherein the reporter protein comprises a β-galactosidase derivative and an amino acid sequence comprising a protease recognition sequence between residues 279-280 of β-galactosidase, and wherein: (i) the recombinant reporter protein retains β-galactosidase activity; (ii) the cleavage of the reporter protein by the protease results in reduced β-galactosidase activity; and (iii) the cleavage of said reporter protein by the protease does not substantially result in accumulation of cleavage products capable of substantially inhibiting said protease.

In one embodiment, the protease recognition sequence is an HCV NS3 cleavage site. According to certain preferred embodiments, the NS3 cleavage site is selected from a group consisting of: NS5A/B, NS3/4A, NS4A/B and NS4B/NS5A. According to other preferred embodiments, the NS3 cleavage site has an amino acid sequence as set forth in any one of SEQ ID NOS:60-63 and 70. In other preferred embodiments, the recombinant reporter protein has an amino acid sequence according to any one of SEQ ID NOS:64-65. According to another embodiment, the invention provides an isolated polypeptide comprising an amino acid sequence as set forth in any one of SEQ ID NOS:64-65.

In another embodiment, there is provided a recombinant reporter gene construct comprising a nucleic acid sequence encoding said reporter protein operably linked to one or more transcription control elements. In another embodiment, the reporter gene construct further comprises a nucleic acid sequence encoding a protease capable of cleaving the recombinant reporter protein and/or a potential inhibitor of said protease, operably linked to one or more transcription control elements.

In one embodiment, the protease is associated with a disease or disorder in a human or non-human subject. In another embodiment, the protease is a viral protease. In a preferred embodiment, the protease is HCV NS3 protease. In another embodiment, the protease is fused to a stabilizing protein. In one preferred embodiment, the stabilizing protein is E. coli maltose binding protein (MBP). In another preferred embodiment, the protease is a recombinant fusion protein comprising NS3, NS4A and MBP. In another preferred embodiment, the protease has an amino acid sequence as set forth in SEQ ID NO:66.

In one embodiment, the potential inhibitors are peptide aptamers, comprising a peptide fused to a stabilizing protein. In a preferred embodiment, the stabilizing protein is E. coli maltose binding protein (MBP). In one preferred embodiment, the peptides are fused to the C terminus of MBP. In another preferred embodiment, the peptides are fused at internal permissive positions of MBP. In another preferred embodiment, the peptides are fused at the internal permissive position following position 133 of MBP.

In another embodiment, the potential inhibitors are antibody fragments including, but not limited to, single-chain antibodies (scFvs) and single antibody domain proteins (dAbs). In another embodiment, the inhibitors are antibody fragments fused to a stabilizing protein. In a preferred embodiment, the stabilizing protein is E. coli maltose binding protein (MBP). In another embodiment, the antibody fragments are fused to the C terminus of MBP. In another embodiment, the potential inhibitors are selected from a library of antibody fragments derived from antibodies isolated from animals immunized against HCV NS3.

In another embodiment, the invention provides an expression vector comprising a reporter gene construct of the invention. According to one preferred embodiment, the vector comprises nucleic acid sequences encoding a recombinant NS3 protease and a recombinant β-galactosidase reporter protein. In another preferred embodiment, the vector has a nucleic acid sequence as set forth in SEQ ID NO:67. In other preferred embodiments, the invention provides an expression vector having a nucleic acid sequence as set forth in SEQ ID NO:68, comprising a nucleic acid sequence encoding a recombinant β-galactosidase reporter protein.

In yet another embodiment, the invention provides a vector having a nucleic acid sequence as set forth in SEQ ID NO:82, useful as a universal platform for insertion of protease-site-coding sequences between lacZ codons 279-280.

In yet another embodiment, the expression vectors containing the reporter gene constructs are contained within a host cell. In a preferred embodiment, the host cell is a bacterial host cell. In another preferred embodiment, the host cell is E. coli. In certain preferred embodiments, the host cell comprises an expression vector having a nucleic acid sequence as set forth in SEQ ID NO:67, comprising nucleic acid sequences encoding a recombinant NS3 protease and a recombinant β-galactosidase reporter protein. In other preferred embodiments, the host cell comprises expression vectors having nucleic acid sequences as set forth in SEQ ID NOS:68 and 69, comprising nucleic acid sequences encoding a recombinant NS3 protease and a recombinant β-galactosidase reporter protein, respectively.

In another aspect, the invention provides a method of screening for protease inhibitors, comprising:
a) co-expressing a protease and a recombinant reporter protein cleavable by the protease in a host cell, wherein the cleavage of the reporter protein by the protease results in reduced activity of said reporter protein, and wherein the cleavage of said reporter protein by the protease does not substantially result in accumulation of cleavage products capable of substantially inhibiting said protease;
b) exposing the host cell to potential inhibitors of said protease; and
c) screening for host cells that retain said reporter protein activity.

In one embodiment, the recombinant reporter gene encodes a β-galactosidase derivative, wherein a protease recognition sequence of said protease is inserted in a permissive site of β-galactosidase so as to retain β-galactosidase activity. In a preferred embodiment, the protease cleavage site is inserted between residues 279-280 of β-galactosidase.

In one embodiment, the protease recognition sequence is an HCV NS3 cleavage site. According to certain preferred embodiments, the NS3 cleavage site is selected from a group consisting of: NS5A/B, NS3/4A, NS4A/B and NS4B/NS5A. According to other preferred embodiments, the NS3 cleavage site has an amino acid sequence as set forth in any one of SEQ ID NOS:60-63 and 70. In other preferred embodiments, the recombinant reporter protein has an amino acid sequence according to any one of SEQ ID NOS:64-65.

In one embodiment, the protease is associated with a disease or disorder in a human or non-human subject. In another embodiment, the protease is a viral protease. In a preferred embodiment, the protease is HCV NS3 protease. In another embodiment, the protease is fused to a stabilizing protein. In one preferred embodiment, the stabilizing protein is E. coli maltose binding protein (MBP). In another preferred embodiment, the protease is a recombinant fusion protein comprising NS3, NS4A and MBP. In another preferred embodiment, the protease has an amino acid sequence as set forth in SEQ ID NO:66.

Potential inhibitors may be expressed by the host cell, either naturally or upon the transformation of suitable constructs encoding them, or may be introduced externally, e.g. by addition of potential inhibitors to the culture medium. The potential inhibitors may include, but are not limited to, peptides or proteins (either recombinant or naturally occurring), nucleic acids or other organic or inorganic compounds (e.g. carbohydrates and polysaccharides).

In one embodiment, the potential inhibitors are peptide aptamers, comprising a peptide fused to a stabilizing protein. In a preferred embodiment, the stabilizing protein is E. coli maltose binding protein (MBP). In one preferred embodiment, the peptides are fused to the C terminus of MBP. In another preferred embodiment, the peptides are fused at internal permissive positions of MBP. In a further preferred embodiment, the peptides are fused at the internal permissive position following position 133 of MBP.

In another embodiment, the potential inhibitors are antibody fragments including, but not limited to, single-chain antibodies (scFvs) and single antibody domain proteins (dAbs). In another embodiment, the inhibitors are antibody fragments fused to a stabilizing protein. In a preferred embodiment, the stabilizing protein is E. coli maltose binding protein (MBP). In another embodiment, the antibody fragments are fused to the C terminus of MBP. In a further embodiment, the potential inhibitors are selected from a library of antibody fragments derived from antibodies isolated from animals immunized against HCV NS3.

In another embodiment, the invention provides protease inhibitors identified by the method of screening disclosed herein.

These and other embodiments of the present invention will become apparent in conjunction with the figures, description and claims that follow.

### BRIEF DESCRIPTION OF THE DRAWINGS

FIGS. 1A-1E show the steps in evaluation of the bacterial genetic screening for NS3 inhibitors and its application for the isolation of inhibitory scFvs. A. Test bacteria that express active NS3 (pMGT14) form white colonies on X-gal supplemented plates while test bacteria that express inactive NS3 (pMGT15) form blue colonies such plates. B. Induction of active MBP-5cNS3 (gray bars), but not of inactive MBP-,scNS3 (black bars) expression with arabinose results in a dose-dependent reduction in β-galactosidase activity as measured by an o-nitrophenyl beta-D-galactopyranoside (ONPG) hydrolysis assay. Error bars represent the standard deviation of the data. C. Evaluation of the dependence of digestion of the engineered β-galactosidase on MBP-sCNSS induction time by an immunoblot. Total protein extracts prepared from non-induced test bacteria (lane 1) or that were induced for 10, 20, 30 60 and 120 min were loaded in lanes 2, 3, 4, 5 and 6, respectively. Extracts prepared from non-induced test bacteria that express the inactive NS3 mutant or induced for 60 and 120 min were loaded in lanes 7, 8 and 9, respectively. Purified β-galactosidase was loaded in the M lane. The upper arrow marks the position of the engineered β-galactosidase while the lower arrow marks the position of MBP-5cNS3. D. Application of the genetic screening for the isolation of NS3-inhibitory scFvs. Affinity selected scFv in pMALc-NN were introduced into test bacteria and plated on X-gal supplemented plates. The arrows mark the positions of a blue and of a white colony. E. A comparison of the color developed by genetic screened bacteria that express four of the inhibitory scFvs on the left, and four control scFvs (Berdichevsky et al., 2003) on the right.

FIGS. 2A-2B demonstrate the evaluation of the selected scFvs binding to NS3 by ELISA. A. Binding curves of the selected scFvs to ,scNS3. Error bars represent the standard deviation of the data. B. Competition ELISA showing the competition of scFvs 171 and 35 as tracers binding to ,ycNS3 by the selected scFvs.

FIGS. 3A-3B demonstrate the evaluation of the selected scFvs inhibition of NS3 catalysis. A. β-galactosidase activity determined by an ONPG hydrolysis by non-induced or isopropyl-beta-d-thiogalactopyranoside (IPTG)-induced genetic screened bacteria carrying selected scFvs. B. In vitro inhibition of NS3 catalysis at two concentrations of tested scFvs. Error bars represent the standard deviation of the data.

FIG. 4 demonstrates the evaluation of the selected peptide aptamers from the NNS 8 and the Rand3 libraries. In vitro inhibition of NS3 catalysis at several concentrations of tested aptamers is presented. Neg is a negative control aptamers (isolated as a white colony) from the Rand3 library.

FIG. 5 exhibits the amino-acid sequence of the engineered substrate (reporter protein): a fusion protein comprised of the 78 N-terminal residues of the E. coli trpR gene product followed by a short linker (both in italics) fused to the 8^{th} codon of the E. coli lacZ gene product with the NS3-cleavable NS5A/B site (in bold type) inserted between residues 279-280 of the E. coli lacZ gene product (original numbering of the E. coli lacZ gene product).

FIG. 6 exhibits the amino-acid sequence of the engineered enzyme: a fusion protein comprised of the E. coli malE gene product (MBP) fused the NS4A peptide (in bold type) followed by the NS3 protease domain derived from the BK strain of HCV

FIGS. 7A-7B exhibit aligned amino-acid sequences of: A. NS3-inhibiting scFvs; the V_{H} and VL (complementarity-determining regions) CDRs are underlined; the linker is in italics; B. Four NS3-inhibiting single-domain antibodies (V_{H}). The CDRs are underlined. These clones were initially identified in the truncated form due to internal stop codons or due to a single base deletion resulting in frameshifting and premature translation termination, which resulted in their expression as single antibody VH domains. They were also characterized after replacing the stop codon with a sense codon, or by adding in the missing base, as they appear as intact scFvs with the corresponding number in FIG. 7A.

FIGS. 8A-8B show spun-cell ELISA of scFvs 35 and 171 cell display analysis. scFvs cell surface displays were evaluated by testing the binding of NS3 to scFv-displaying bacteria at 4 different IPTG concentrations: 0, 0.001, 0.01 and 0.1 mM. A. scFv-35 (black bars) and scFv-H23 as negative control (gray bars). B. scFv-171 (black bars) and scFv-CD30 as negative control (gray bars). Error bars represent the standard deviation of the data.

FIGS. 9A-9B demonstrate Dot-Blot results of scFv-171 binders from DIP output of FDCl 2C library. A. 192 single colonies were screened (96 on each filter). White circles mark the phages that were suspected as binders and were verified by another Dot-Blot analysis. Black circles mark the phages that were picked as negative control for the verifying Dot-Blot. B. Dot-Blot analysis for verifying the suspected binders from the first Dot-Blot. Phages were loaded longitudinally in secondary dilutions. Column F3, D14 and D21 were loaded with the negative control phages.

FIGS. 10A-10B demonstrate Dot-Blot results of scFv-35 binders from DIP output of FM7 and FDC 12C mixed libraries. A. 192 single colonies were screened. White circles mark the phages that were suspected as binders and were verified by another Dot-Blot analysis. Black circles mark the phages that were picked as negative control for the verifying Dot-Blot. B. Dot-Blot analysis for verifying the suspected binders from the first Dot-Blot. Phages were loaded longitudinally in secondary dilutions. Column F9 and E13 were loaded with the negative control phages.

FIGS. 11A-11B demonstrate Dot-Blot results of scFv-35 binders from panning output of FM7 and FDC 12C mixed libraries. A. 192 single colonies were screened. White circles mark the phages that were suspected as binders and were verified by another Dot-Blot analysis. Black circles mark the phages that were picked as negative control for the verifying Dot-Blot. B. Dot-Blot analysis for verifying the suspected binders from the first Dot-Blot. Phages were loaded longitudinally in secondary dilutions. Column A6 and B15 were loaded with the negative control phages.

FIGS. 12A-12B demonstrate RasTop representation of scFvs 35 and 171 predicted epitopes on NS3. NS3 is shown as a ribbon representation. The catalytic triad is shown as space-fill residues in gray. A. scFv-35 epitope on NS3 is shown as space-fill residues in black: residues 97-102, 147, 148—CTCGSS, TG-. B. scFv-171 epitope on NS3 is shown as space-fill residues in black: residues 95, 97-103, 148, 149-T, CTCGSSA, GH.

FIG. 13 demonstrates the shared epitope of scFvs 35 and 171 on NS3. NS3 is shown as a ribbon representation. The catalytic triad is shown as space-fill residues in gray at the bottom. The shared epitopes residues shown as space-fill in gray. The two cysteines and the histidine residues that are part of the enzyme zinc-binding site are marked by arrows. Residues that are unique to the mapped epitope of each antibody (not shared) are circled and marked by arrows.

FIG. 14A-14B show the ScFvs 35 and 171 competitive in vitro assays with epitope mimetic peptide. A. Evaluation of MBP-scFv 35 without peptide (white bars), in the presence of the epitope-mimetic peptide (gray bars), in the presence of control peptide X (horizontal line bars), in the presence of control peptide 6 (diagonal line bars). B. Evaluation of MBP-scFv 171 without peptide (doted bars), in the presence of the epitope-mimetic peptide (gray bars), in the presence of control peptide X (horizontal line bars), in the presence of control peptide 6 (diagonal line bars). C. Controls: evaluation of MBP-scFv 35 without peptide (white bars), MBP-scFv 171 without peptide (doted bars), the epitope-mimetic peptide without scFv (gray bars), the control peptide X without scFv (horizontal line bars), the control peptide 6 without scFv (diagonal line bars). Error bars represent the standard deviation of the data.

FIG. 15 demonstrates the inhibition of HCV RNA replicon amplification. Activity of SEAP secreted from Huh7 cells supporting replication of HCV RNA replicons over successive 24-h intervals following transient transfection with intrabodies.

### DETAILED DESCRIPTION OF THE INVENTION

The present invention provides novel NS3 serine protease inhibitors, analogs, fragments and derivatives thereof, nucleic acids encoding same, and methods of use thereof for the treatment of HCV infection. The invention further provides novel constructs and methods for the screening of protease inhibitors in vivo.

The present invention is based in part on the generation of a novel screening for inhibitors of NS3 catalysis. The inventors constructed a genetic screening based on the concerted co-expression of a reporter gene, of recombinant NS3 and of stabilized potential inhibitors, in a host cell. The reporter system was constructed by inserting a peptide corresponding to the NS5A/13 cleavage site of NS3 into a permissive site of the enzyme β-galactosidase, with NS3 expressed from the same plasmid. The resultant β-galactosidase enzyme is active, conferring a Lac⁺ phenotype that is lost upon induction of NS3 expression. The identification of inhibitors, demonstrated herein by isolating NS3 inhibiting single-chain antibodies (scFvs), single-domain antibodies (dAbs) or peptide aptamers, expressed from a compatible plasmid, is based on the appearance of blue colonies (NS3 inhibited) on the background of colorless colonies (NS3 active) on X-gal indicative plates. Surprisingly, upon the intracellular cleavage of the engineered β-galactosidase by NS3 in E. coli, the resultant cleavage products were found to undergo proteolytic degradation, thus preventing potential obscuring by product inhibition of NS3 catalysis. The putative inhibitors were stabilized by their fusion to maltose binding protein (MBP), resulting in increased intracellular inhibitor concentrations. Thus, the constructed assay was characterized as a highly sensitive screening method capable of identifying NS3 inhibitors that escape detection by in vitro approaches, and is highly advantageous compared to other screening methods that due to accumulation of cleavage products, may be subject to product inhibition (to that affect, NS3 itself is a protease subject to product inhibition).

Constructs and Methods of Screening for Protease Inhibitors

According to certain aspects, the invention provides novel constructs and methods for the screening of protease inhibitors in vivo, using a recombinant engineered reporter protein that is cleavable by a protease, co-expressed with the protease in a host cell. The proteolytic cleavage of the reporter protein does not result in accumulation of cleavage products in the host cell, thus enabling a highly sensitive, high throughput screening method which is preferable to other screening methods compromised by product inhibition.

In one embodiment, the invention provides an isolated polynucleotide comprising a nucleic acid sequence encoding a recombinant reporter protein, wherein the reporter protein comprises a β-galactosidase derivative and an amino acid sequence comprising a protease recognition sequence between residues 279-280 of β-galactosidase, and wherein: (i) the recombinant reporter protein retains β-galactosidase activity; (ii) the cleavage of the reporter protein by the protease results in reduced β-galactosidase activity; and (iii) the cleavage of said reporter protein by the protease does not substantially result in accumulation of cleavage products capable of substantially inhibiting said protease.

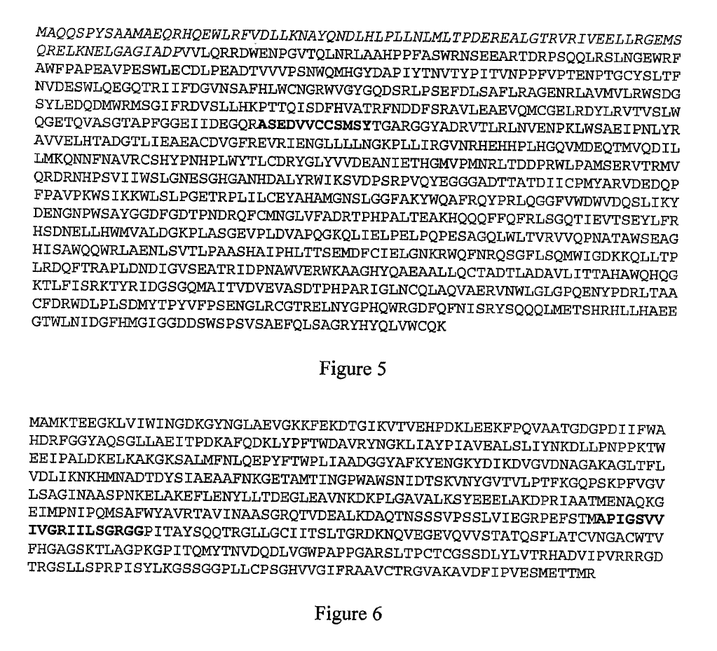

In one embodiment, the protease recognition sequence is an HCV NS3 cleavage site. According to certain preferred embodiments, the NS3 cleavage site is selected from a group consisting of: NS5A/B, NS3/4A, NS4A/B and NS4B/NS5A, which may vary for different HCV genotypes and subtypes (Kwong et al., 1998). According to other preferred embodiments, the NS3 cleavage site is a cleavage site derived from the Ib subtype HCV BK strain, selected from a group consisting of: NS5A/B (EDWCCSMSY, SEQ ID NO:60, and ASEDWCCSMSY SEQ ID NO:70), NS3/4A (DLEWTSTWV, SEQ ID NO:61), NS4A/B (DEMEECASHL, SEQ ID NO:62) and NS4B/NS5A (DCSTPCSGSW, SEQ ID NO:63). In other preferred embodiments, the recombinant reporter protein has an amino acid sequence according to any one of SEQ ID NOS:64-65 presented in FIG. 5, wherein the amino acid sequence of the engineered substrate is denoted as SEQ ID NO: 64 and the amino acid sequence of the engineered substrate without the preceding N-terminal residues corresponding to the E. coli TrpR gene product and the linker, is denoted as SEQ ID NO:65. According to another embodiment, the invention provides an isolated polypeptide comprising an amino acid sequence as set forth in any one of SEQ ID NOS:64-65.

In another embodiment, there is provided a recombinant reporter gene construct comprising a nucleic acid sequence encoding said reporter protein operably linked to one or more transcription control elements. In another embodiment, the reporter gene construct further comprises a nucleic acid sequence encoding a protease capable of cleaving the recombinant reporter protein and/or a potential inhibitor of said protease, operably linked to one or more transcription control elements, as will be described hereinbelow.

In another aspect, the invention provides a method of screening for protease inhibitors, comprising:
a) co-expressing a protease and a recombinant reporter protein cleavable by the protease in a host cell, wherein the cleavage of the reporter protein by the protease results in reduced activity of said reporter protein, and wherein the cleavage of said reporter protein by the protease does not substantially result in accumulation of cleavage products capable of substantially inhibiting said protease;
b) exposing the host cell to potential inhibitors of said protease; and
c) screening for host cells that retain said reporter protein activity.

In another aspect, the invention provides protease inhibitors identified by the method of screening disclosed herein.

Suitable embodiments for the production and use of the constructs and methods of the present invention in identifying and isolating protease inhibitors are disclosed in detail herein.

1. Construction of the Genetic Screening

Reporter genes. In one embodiment, the recombinant reporter gene encodes a galactosidase derivative, wherein a protease recognition sequence of said protease is inserted in a permissive site of β-galactosidase so as to retain β-galactosidase activity. In a preferred embodiment, the protease cleavage site is inserted between residues 279-280 of β-galactosidase.

The term “β-galactosidase activity” as used herein indicates the ability of the reporter protein to hydrolyze lactose yielding glucose and galactose, allowing utilization of lactose as a carbon source. The enzymatic activity of β-galactosidase may be detected by utilizing suitable color indicator compounds, including, but not limited to X-gal and Fast Blue RR, which, upon their cleavage by β-galactosidase, produce a detectable color. Thus, for example, bacteria expressing said reporter protein appear as blue colonies when grown on a semi-solid medium-containing X-gal. Other substrates that may be used for detecting β-galactosidase activity include, but are not limited to, substrates that upon cleavage result in a fluorescent signal, e.g. Fluorescein-β-D-Galactopyranoside (FDG; commercially available from Marker Gene Technologies, Inc.), or in signals measurable by amperometric sensors with substrates such as PAPG

The term “protease recognition sequence” refers to a consecutive amino acid sequence that is recognized by a protease of interest and is required for the proteolytic cleavage. A protease recognition sequence may be coincident with the protease cleavage site (i.e., the site at which the cleavage by the protease occurs). That is, the protease recognition sequence may include one or more amino acids on either side of the peptide bond to be hydrolyzed by the protease. Alternatively, the protease recognition sequence may be one, two or more amino acids distal, at the amino or carboxy terminus, to the cleavage site of the protease, as long as the cleavage of said reporter protein by said protease does not substantially result in accumulation of cleavage products capable of substantially inhibiting said protease. In certain embodiments, a protease recognition sequence of the present invention is derived from the amino acid sequence of a naturally occurring substrate of the protease of interest.

In one embodiment, the protease recognition sequence is an HCV NS3 cleavage site. According to certain preferred embodiments, the NS3 cleavage site is selected from a group consisting of: NS5A/B, NS3/4A, NS4A/B and NS4B/NS5A. According to other preferred embodiments, the NS3 cleavage site has an amino acid sequence as set forth in any one of SEQ ID NOS:60-63 and 70. In other preferred embodiments, the recombinant reporter protein has an amino acid sequence according to any one of SEQ ID NOS:64-65.

Other reporter genes may also be used, as long as: (i) the reporter protein is capable of being assayed for a change in color, light or activity when combined with a protease enzyme in a host cell; (ii) the introduction of the protease recognition site does not eliminate the activity of the reporter protein, so that the fusion protein can be used in an assay system to screen for inhibitors of said protease; and (iii) the cleavage of said reporter protein by said protease does not substantially result in accumulation of cleavage products capable of substantially inhibiting said protease.

When the protease enzyme is added to the resulting reporter protein, the protease cleaves at the cleavage site, thereby resulting in reduced activity of said reporter protein. The phrases “reduced activity of a reporter protein” and “substantially inhibiting a protease” refer to a detectable reduction in the activity of the reporter protein monitored by means of, for example, color change, light change or other known methods of monitoring the enzymatic activity. When determining whether the cleavage of a reporter gene by a protease substantially results in the accumulation of cleavage products capable of substantially inhibiting the protease, in addition to the functional assays for protease activity described above, lysates of host cells in which the reporter protein and the protease are expressed may be assayed for degradation products of the reporter protein. This may be achieved, for example, using antibodies recognizing the reporter protein, by methods well known in the art.

Examples for other reporter genes that may be utilized to generate a recombinant reporter gene of the invention include, but are not limited to: alkaline phosphatase, β-glucuronidase, acetyltransferase, luciferase, green fluorescent protein, red fluorescent protein, aequorin, chloramphenicol acetyl transferase and horseradish peroxidase.

Proteases. The present invention may be used to screen for inhibitors of any protease of interest, including naturally occurring proteases, naturally occurring variants of wild type proteases, and artificially mutagenized proteases, as long as they are enzymatically active.

In one embodiment, the protease is associated with a disease or disorder in a human or non-human subject. Exemplary proteases include those that have been implicated in human diseases, e.g. trypsin and elastase that are involved in the onset of emphysema, and renin which has been implicated in hypertension. Other exemplary proteases are essential for the replication of microbial pathogens (e.g., HCV, poliovirus and HIV proteases), or involved in the destructive effects of microbial pathogens in ways that do not involve replicative processes (e.g., collagenases from Clostridium histolylicum that participate in the invasiveness of the bacterium by destroying the connective tissue barriers of the host).

In one embodiment, the protease is a viral protease. In a preferred embodiment, the protease is HCV NS3 protease, which sequence may vary for different HCV genotypes and subtypes.

In another embodiment, the protease is fused to a stabilizing protein. In a preferred embodiment, the stabilizing protein is E. coli maltose binding protein (MBP). In another preferred embodiment, the protease is a recombinant fusion protein comprising NS3, NS4A and MBP. In another preferred embodiment, the protease has an amino acid sequence as set forth in SEQ ID NO:66.

Recombinant constructs and transcription control elements. The present invention further provides recombinant constructs, comprising reporter genes, proteases and/or potential inhibitors operably linked to transcription control elements, i.e. nucleic acid sequences regulating their expression in a host cell. For a prokaryotic host cell, such transcription control elements include promoters, optionally containing operator portions. The phrase “operably linked” refers to linking a nucleic acid sequence to a transcription control element in a manner such that the molecule can be expressed when transformed, transfected or transduced into a host cell.

Various prokaryotic transcription control elements are known in the art, including, but not limited to, the lac, tac, trpR, araBAD, recA, Tl, λPR and λP_{L} promoters. The reporter genes, proteases and potential inhibitors may be controlled by the same transcription control element or by different transcription control elements. Advantageously, the genes are operably linked to different transcription control elements, thus enabling a differential regulation of their expression levels and an inducible expression when desired. One non-limitative approach is demonstrated in the Examples, in which the lacZ reporter was subcloned under the control of the trpR promoter, the NS3 protease was subcloned under the control of the araBAD promoter, and potential inhibitors were subcloned under tac promoter. Thus, three different regulatory mechanisms for expression were utilized: IPTG-inducible high-level expression of potential inhibitors, arabinose-inducible medium level NS3 expression and constitutive low level of β-galactosidase expression. The system is thus tuned such as sufficient protease is made to fully digest the engineered reporter gene, unless inhibited by an excess of inhibitor that is produced at still a higher concentration.

Vectors. In one embodiment, there is provided an expression vector comprising at least one nucleic acid sequence encoding a polypeptide or peptide selected from: a protease of the invention, a reporter protein of the invention and a potential protease inhibitor of the invention, operably linked to one or more transcription control elements.

An “expression vector” refers to a nucleic acid molecule capable of replication and expressing a gene of interest when transformed, transfected or transduced into a host cell. The expression vectors comprise one or more phenotypic selectable markers and an origin of replication to ensure maintenance of the vector and to, if desired, provide amplification within the host. Selectable markers include, for example, sequences conferring antibiotic resistance markers, which may be used to obtain successful transformants by selection, such as ampicillin, tetracycline and kanamycin resistance sequences, or supply critical nutrients not available from complex media. Suitable expression vectors may be plasmids derived, for example, from pBR322 or various pUC plasmids, which are commercially available. Other expression vectors may be derived from bacteriophage, phagemid, or cosmid expression vectors, all of which are described in sections 1.12-1.20 of Sambrook et al., (Molecular Cloning: A Laboratory Manual. 3^{rd} edn., 2001, Cold Spring Harbor Laboratory Press). Isolated plasmids and DNA fragments are cleaved, tailored, and ligated together in a specific order to generate the desired vectors, as is well known in the art (see, for example, Sambrook et al., ibid).

According to one preferred embodiment, the vector comprises nucleic acid sequences encoding a recombinant NS3 protease and a recombinant β-galactosidase reporter protein. In another preferred embodiment, the vector is pMGT14 (see Example 1), having a nucleic acid sequence as set forth in SEQ ID NO:67. In other preferred embodiments, the invention provides an expression vector having a nucleic acid sequence as set forth in SEQ ID NO: 68 (pEB13-/acZ_{N}s₅A/B, see Examples), comprising a nucleic acid sequence encoding a recombinant β-galactosidase reporter protein.

In yet another embodiment, the invention provides a vector having a nucleic acid sequence as set forth in SEQ ID NO:82 (pEB13-Sfi, see Examples), useful as a universal platform for insertion of protease-site-coding sequences between lacZ codons 279-280.

Host cells. In yet another embodiment, the expression vector comprising the nucleic acid sequence is contained within a host cell. In a preferred embodiment, the host cell is a bacterial host cell. In another preferred embodiment, the host cell is E. coli. Other suitable prokaryotic hosts include, but are not limited to: Bacillus subtilis, Salmonella typhimurium and various species within the genera Pseudomonas, Streptomyces, and Staphylococcus. In other embodiments, eukaryotic host cells are used, including, but not limited to plant cells, yeast cells, insect cells or animal cells, with suitable vectors and expression control elements known in the art.

Prokaryotic host cells or other host cells with rigid cell walls are preferably transformed using the calcium chloride method as described in section 1.82 of Sambrook et al., (ibid). Alternatively, electroporation may be used for transformation of these cells. Prokaryote transformation techniques are known in the art, e.g. Dower, W. J., in Genetic Engineering, Principles and Methods, 12:275-296, Plenum Publishing Corp., 1990; Hanahan et al., Meth. Enzymol., 204:63 1991.

In certain preferred embodiments, the host cell comprises an expression vector having a nucleic acid sequence as set forth in SEQ ID NO: 67, comprising nucleic acid sequences encoding a recombinant NS3 protease and a recombinant β-galactosidase reporter protein. In other preferred embodiments, the host cell comprises expression vectors having nucleic acid sequences as set forth in SEQ ID NOS:68 and 69 (see Examples) comprising nucleic acid sequences encoding a recombinant NS3 protease and a recombinant β-galactosidase reporter protein, respectively.

2. Screening and Identification of Inhibitors.

Host cells expressing the reporter protein and protease of the invention are exposed to potential inhibitors of the protease. Next, the host cell population is screened for cells that retain reporter protein activity.

The term “exposed” is used herein to indicate that a host cell is contacted with a potential protease inhibitor such that the inhibitor can effectively inhibit the protease. Potential inhibitors may be expressed by the host cell, either naturally or upon the transformation of suitable constructs encoding them, or may be introduced externally, e.g. by addition of potential inhibitors to the culture medium. A potential inhibitor is identified as an inhibitor of the protease if the reporter protein is not cleaved and remains active when monitored by means of, for example, color change, light change or other known methods of monitoring the enzymatic activity, as described above. Host cells exposed to a functional inhibitor can thus be distinguished from host cells expressing an uninhibited protease, and are considered to “retain reporter protein activity”.

Inhibitors and libraries. A potential inhibitor may be a single compound of interest or a member of a library of potential inhibitors. For example, a library of potential inhibitors may be a synthetic combinatorial library (e.g., a combinatorial chemical library), a cellular extract, a bodily fluid (e.g., urine, blood, tears, sweat, or saliva), or other mixture of synthetic or natural products (e.g., a library of small molecules or a fermentation mixture). A library of potential inhibitors can include, for example, amino acids, peptides, polypeptides, proteins (including, but not limited to, antibodies, antibody fragments and peptide aptamers), or fragments of peptides or proteins; nucleic acids (e.g., DNA; RNA; or peptide nucleic acids, PNA); aptamers; or compounds such as carbohydrates and polysaccharides. Each member of the library can be singular or can be a part of a mixture (e.g., a compressed library). The library can contain purified compounds or can be “dirty” (i.e., containing a significant quantity of impurities).

Commercially available libraries (e.g., from Affymetrix, ArQuIe, Neose Technologies, Sarco, Ciddco, Oxford Asymmetry, Maybridge, Aldrich, Panlabs, Pharmacopoeia, Sigma, or Tripose) can also be used with the new methods.

In addition to libraries of potential inhibitors, special libraries called diversity files can be used to assess the specificity, reliability, or reproducibility of the new methods. Diversity files contain a large number of compounds (e.g., 1000 or more small molecules) representative of many classes of compounds that could potentially result in nonspecific detection in an assay. Diversity files are commercially available or can also be assembled from individual compounds commercially available from the vendors listed above.

Fusion-stabilized peptides (peptide aptamers). Peptide aptamers represent a novel generation of molecules in which variable peptides are inserted into a protein scaffold. As such, they can bind to their target in vivo and have the potential to selectively block its activity. Several bacterial proteins had been recently applied as scaffolds for peptide aptamers, including thioredoxin, staphylococcal nuclease and alpha-amylase, as well as non-bacterial proteins such as green fluorescent protein (Colas, 2000) (Hoppe-Seyler and Butz, 2000). The scaffold share intrinsic stability making it possible to express peptide aptamers in vivo at high concentrations, and, having the peptide aptamer been identified, utilize its high level expression and easy purification for subsequent analysis. Once identified, a peptide aptamer may be evaluated as a free peptide, where in some cases it is as active as in the context of the aptamer (Hoppe-Seyler and Butz, 2000). Moreover, small synthetic molecules may be derived from such bioactive aptamers to form the basis of new therapeutics.

The E. coli maltose binding protein (MBP) has not been applied before as a scaffold for peptide aptamers before. The inventors chose MBP as a scaffold for the fusion-stabilized peptide aptamers for several reasons. MBP is produced at very high levels in E. coli from which it can be recovered and purified by a single-step affinity chromatography on amylose columns. MBP confers stability on peptides and proteins that are fused to it, as was reported by the inventors and others (Bach et al., 2001). Peptides may be linked at the C-terminus of MBP in a linear (unconstrained) form, or structurally (conformationally) constrained in internal positions of MBP that are permissive to peptide insertion (Martineau et al., 1996). It was reported that in some cases, constrained peptides might prove better binders than unconstrained ones (Colas 2000).

In one embodiment, the potential inhibitors are peptide aptamers, comprising a peptide fused to a stabilizing protein. In a preferred embodiment, the stabilizing protein is E. coli maltose binding protein (MBP). In one preferred embodiment, the peptides are fused to the C terminus of MBP. In another preferred embodiment, the peptides are fused at internal permissive positions of MBP. In another preferred embodiment, the peptides are fused at the internal permissive position following position 133 of MBP.

Recombinant antibody fragments. Antibodies are versatile immunological reagents used for a variety of diagnostic and therapeutic applications. Traditional monoclonal or polyclonal antibodies are divalent and are highly useful because of their specific and high-affinity binding to antigen. However, small antibody fragments are proving to have the same utility. The advent of recombinant techniques has allowed for the generation of monovalent synthetic antibody fragments, such as single-chain antibodies (scFvs) and Fab fragments that lack a portion or all of the antibody constant domains normally found in an intact antibody. Single-chain antibodies are small recognition units consisting of the variable regions of the immunoglobulin heavy (VH) and light (VL) chains which are connected by a synthetic linker sequence. Single antibody domain proteins (dAbs) are minimized antibody fragments comprising either an individual VL domain or an individual VH domain. The smaller size of antibody fragments compared to whole antibodies is advantageous to applications requiring, e.g. tissue penetration or rapid blood clearance.

Antibodies and antibody fragments may be particularly advantageous as protease inhibitors. For example, inhibition of NS3 catalysis by small molecule inhibitors has been a formidable challenge due to featureless appearance of the NS3 catalytic groove that results in weak binding; antibodies may offer a better solution by forming a “clamp” that involves interactions with NS3 that are located away from the active site on one hand, and blocking or interfering with substrate binding on the other. In addition, it has been recently shown that recombinant antibody fragments, when synthesized within a living cell and targeted to a particular subcellular compartment, can be used to specifically interfere with the normal trafficking of the target molecule, thereby preventing it from accumulating at its normal destination. The inventors have recently reported the isolation of non-neutralizing anti-NS3 scFvs by antibody phage-display, that as intracellular antibodies (intrabodies) partially inhibited NS3-mediated cell transformation by diverting NS3 from the cell cytoplasm to the nucleus (Zemel et al., 2004). The studies of intrabody-mediated phenotypic modulation or prevention of cell transformation demonstrated that fine-tuning of the desired phenotype can be achieved by directing the intrabodies into various compartments within the cell (Marasco, 1997).

In another embodiment, the potential inhibitors are antibody fragments including, but not limited to, single-chain antibodies (scFvs) and single antibody domain proteins (dAbs). In another embodiment, the inhibitors are antibody fragments fused to a stabilizing protein. In a preferred embodiment, the stabilizing protein is E. coli maltose binding protein (MBP). In another embodiment, the antibody fragments are fused to the C terminus of MBP. In another embodiment, the potential inhibitors are selected from a library of antibody fragments derived from antibodies isolated from animals immunized against HCV NS3.

The investigation of the properties of antibodies and their application as intrabodies require efficient tools to generate and isolate these molecules. Phage display technology paved the way for antibody engineering as a tool to generate and characterize antibodies and in parallel gain access to the genes that encode for them (Benhar 2001). Recombinant antibody fragments may be isolated from phage libraries by affinity selection or more advanced approaches such as DIP selection (Benhar et al., 2000).

Isolation of Protease Inhibitors.

According to certain embodiments, various methods may optionally be used to enrich for protease inhibitors prior to the screening step. The enrichment can be preformed before or after exposing the host cells to the potential protease inhibitors.

For instance, when the inhibitors are expressed within the host cells, the host cell population may be enriched for inhibitor expressing clones. A non-limitative approach is described herein, in which a β-galactosidase derivative is used as a reporter protein in E. coli. Upon inducing the expression of the potential protease inhibitors, the cells are initially grown on lactose as a sole carbon source; the enrichment is brought about by virtue of faster growth rate of protease-inhibited clones.

Other examples may include enriching a library for protease binders in vitro, e.g. by affinity selection.

Screening for protease inhibitors is performed as described above, by identifying host cells which retain protease activity measured using e.g. color indicator compounds or light emitting reactions.

The potency of the inhibitors isolated by the genetic screening may optionally be further determined in vitro. The affinity of the isolated inhibitors to the protease may be determined e.g. by ELISA. Their ability to inhibit protease catalysis may be quantified and their IC50 determined using e.g. fluorometric assays as described herein.

Novel NS3 Inhibitors

In one aspect, the present invention provides novel NS3 inhibitors. In one embodiment, the inhibitor is a single-chain antibody (scFv) having an amino acid sequence as set forth in any one of SEQ ID NOS: 1-11. In another embodiment, the inhibitor is a single antibody domain protein (dAb) derived from the isolated scFv inhibitors of the invention. In another embodiment, the inhibitor is a dAb having an amino acid sequence as set forth in any one of SEQ ID NOS: 12-14 and 113.

In another embodiment, the scFv or dAb is fused to a stabilizing protein. In a preferred embodiment, the stabilizing protein is E. coli maltose binding protein (MBP). In another embodiment, the scFv or dAb is fused to the C terminus of MBP. According to another embodiment, the inhibitor is a scFv fused to the C terminus of MBP, having an amino acid sequence as set forth in any one of SEQ ID NOS: 15-25. In another embodiment, the inhibitor is a dAb fused to the C terminus of MBP, having an amino acid sequence as set forth in any one of SEQ ID NOS:26-28 and 114. Other embodiments include fragments, homologs, analogs and derivatives thereof, as will be described hereinbelow.

In another embodiment, the inhibitor is a peptide having an amino acid sequence as set forth in any one of SEQ ID NOS:29-35. In another embodiment, the inhibitor is a peptide aptamer, comprising a peptide inhibitor of the invention fused to a stabilizing protein. In a preferred embodiment, the stabilizing protein is E coli maltose binding protein (MBP). In one preferred embodiment, the peptide is fused to the C terminus of MBP. In another preferred embodiment, the peptide is fused at internal permissive positions of MBP. In another preferred embodiment, the peptide is fused at the internal permissive position following position 133 of MBP. According to another embodiment, the inhibitor is a free peptide derived from a peptide aptamer. In another embodiment, the peptide aptamer has an amino acid sequence as set forth in any one of SEQ ID NOS:36-49. Other embodiments include fragments, homologs, analogs and derivatives thereof, as will be described hereinbelow.

According to other embodiments, the invention comprises nucleic acids encoding the NS3 inhibitors of the invention, as well as recombinant constructs, expression vectors and pharmaceutical compositions thereof, as described hereinbelow.

Nucleic Acids

As used herein the terms “nucleic acid sequence”, “oligonucleotide” or “polynucleotide” refer to polymers of deoxyribonucleotides, ribonucleotides, and modified forms thereof in the form of a separate fragment or as a component of a larger construct, in a single strand or in a double strand form. The DNA or RNA molecules may be complementary DNA (cDNA), genomic DNA, synthesized DNA or a hybrid thereof or an RNA molecule such as mRNA. Accordingly, as used herein, the terms “DNA construct”, “gene construct”, “nucleic acid sequence”, “polynucleotide” and “oligonucleotide” are meant to refer to both DNA and RNA molecules. The term “oligonucleotide” refers to a polymer having not more than 50 nucleotides while the term “polynucleotide” refers to a polymer having more than 50 nucleotides. The term “nucleic acid sequence” refers to both oligonucleotide sequence and polynucleotide sequence. The terms nucleic acid sequence, oligonucleotide sequence and polynucleotide sequence are used in the invention interchangeably.

An isolated nucleic acid sequence can be obtained from its natural source, either as an entire (i.e., complete) gene or a portion thereof. A nucleic acid molecule can also be produced using recombinant DNA technology (e.g., polymerase chain reaction (PCR) amplification, cloning) or chemical synthesis (see e.g. Sambrook et al., 2001; Ausubel, et al., 1989, Chapters 2 and 4). Nucleic acid sequences include natural nucleic acid sequences and homologs thereof, including, but not limited to, natural allelic variants and modified nucleic acid sequences in which nucleotides have been inserted, deleted, substituted, and/or inverted in such a manner that such modifications do not substantially interfere with the nucleic acid molecule's ability to encode a functional polypeptide or peptide of the invention.

A polynucleotide or oligonucleotide sequence can be deduced from the genetic code of a protein; however, the degeneracy of the code must be taken into account. Nucleic acid sequences of the invention include sequences, which are degenerate as a result of the genetic code, which sequences may be readily determined by those of ordinary skill in the art.

The oligonucleotides or polynucleotides of the invention may contain a modified internucleoside phosphate backbone to improve the bioavailability and hybridization properties of the oligonucleotide or polynucleotide. Linkages are selected from the group consisting of phosphodiester, phosphotriester, methylphosphonate, phosphoroselenoate, phosphorodiselenoate, phosphoroanilothioate, phosphoroanilidate, phosphoramidate, phosphorothioate, phosphorodithioate or combinations thereof.

Additional nuclease linkages include alkylphosphotriester such as methyl- and ethylphosphotriester, carbonate such as carboxymethyl ester, carbamate, morpholino carbamate, 3′-thioformacetal, silyl such as dialkyl (C1-C6)- or diphenylsilyl, sulfamate ester, and the like. Such linkages and methods for introducing them into oligonucleotides are described in many references, e.g. reviewed generally by Peyman and Ulmann, Chemical Reviews, 90:1543-584 (1990).

In another aspect, the invention provides constructs and vectors comprising nucleic acid sequences encoding the protease inhibitors of the invention. Such constructs and vectors may be utilized for the expression and production of polypeptide- or peptide-based protease inhibitors. These constructs may also be used for gene therapy, by expressing the protease inhibitors in a subject in need thereof.

A polynucleotide or oligonucleotide sequence can be deduced from the genetic code of a polypeptide or peptide; however, the degeneracy of the code must be taken into account. Nucleic acid sequences of the invention also include sequences, which are degenerate as a result of the genetic code, which sequences may be readily determined by those of ordinary skill in the art.

Gene constructs suitable for expressing the protease inhibitors of the invention in a subject in need thereof comprise a nucleic acid sequence which encodes the protease inhibitor and which includes initiation and termination signals operably linked to regulatory elements including a promoter (and optionally enhancer and polyadenylation signal sequences required for expression in eukaryotic systems) capable of directing expression in the cells of a subject. Thus, a gene construct contains the necessary regulatory elements operably linked to the nucleic acid sequence that encodes a protease inhibitor, such that when present in a cell of the individual, the protease inhibitor sequence will be expressed.

Suitable transcription control elements include any transcription control sequence that can function in at least one of the recombinant cells of the present invention. A variety of such transcription control elements are known to those skilled in the art. Preferred transcription control elements include those, which function in animal, bacteria, helminthes, insect cells, and preferably in animal cells. For expression in animal cells, preferable transcription control elements include, but are not limited to RSV control sequences, CMV control sequences, retroviral LTR sequences, SV-40 control sequences and β-actin control sequences as well as other sequences capable of controlling gene expression in eukaryotic cells. Additional suitable transcription control elements include tissue-specific promoters and enhancers (e.g., liver specific enhancers and promoters).

The present invention is further related to an expression vector comprising the recombinant constructs of the present invention. Suitable eukaryotic expression vector is for example: pcDNA3, pcDNA3.1 (+/−), pZeoSV2(+/−), ρSecTag2, pDisplay, pEF/myc/cyto, pCMV/myc/cyto, pCR3.1, pCI, pBK-RSV, pBK-CMV, pTRES or their derivatives.

According to the present invention, a host cell can be transfected in vivo (i.e., in an animal) or ex vivo (i.e., outside of an animal). Transfection of a nucleic acid molecule into a host cell can be accomplished by any method by which a nucleic acid molecule can be inserted into the cell. Transfection techniques include, but are not limited to, transfection, electroporation, microinjection, lipofection, adsorption, and protoplast fusion. Preferred methods to transfect host cells in vivo include lipofection and adsorption. Preferred host cells include liver cells of a liver transplant.

A host cell may also be infected in vivo or ex vivo by a viral vector comprising the nucleic acid molecules of the present invention. A viral vector includes an isolated nucleic acid molecule useful in the present invention, in which the nucleic acid molecules are packaged in a viral coat that allows entrance of DNA into a cell. A number of viral vectors can be used, including, but not limited to, those based on alphaviruses, poxviruses, adenoviruses, herpesviruses, lentiviruses, adeno-associated viruses (AAV), bacteriophages, virus-like particles (VLPs) and retroviruses. Recombinant adenoviruses have several advantages over retroviral and other viral-based gene delivery methods. Adenoviruses have never been shown to induce tumors in humans and have been safely used as live vaccines. Adenovirus does not integrate into the human genome as a normal consequence of infection, thereby greatly reducing the risk of insertional mutagenesis possible with retrovirus or AAV vectors. This lack of stable integration also leads to an additional safety feature in that the transferred gene effect will be transient, as the extra-chromosomal DNA will be gradually lost with continued division of normal cells. Stable, high titer recombinant adenovirus can be produced at levels not achievable with retrovirus or AAV, allowing enough material to be produced to treat a large patient population.

It may be appreciated by one skilled in the art that use of recombinant DNA technologies can improve expression of transfected nucleic acid molecules by manipulating, for example, the number of copies of the nucleic acid molecules within a host cell, the efficiency with which those nucleic acid molecules are transcribed, the efficiency with which the resultant transcripts are translated, and the efficiency of post-translational modifications. Recombinant techniques useful for increasing the expression of nucleic acid, molecules of the present invention include, but are not, limited to, operatively linking nucleic acid molecules to high-copy number plasmids, integration of the nucleic acid molecules into one or more host cell chromosomes, addition of vector stability sequences to plasmids, substitutions or modifications of transcription control signals (e.g., promoters, operators, enhancers), substitutions or modifications of translational control signals (e.g., ribosome binding sites, Shine-Dalgarno sequences), modification of nucleic acid molecules of the present invention to correspond to the codon usage of the host cell, and deletion of sequences that destabilize transcripts.

Proteins, Peptides and Derivatives

The polypeptides and peptides of the invention may be synthesized using any recombinant or synthetic method known in the art. A non-limitative example for recombinant production of NS3 inhibitors is presented in the Examples; however, other synthesis methods may be used, including, but not limited to, solid phase (e.g. Boc or f-Moc chemistry) and solution phase synthesis methods. For solid phase peptide synthesis, a summary of the many techniques may be found in Meienhofer, 1973.

The amino acid residues described herein are preferred to be in the “L” isomeric form. However, residues in the “D” isomeric form can be substituted for any L-amino acid residue, as long as the peptide retains the desired functional property.

It should be understood that an NS3 inhibitor need not be identical to the amino acid sequence of the NS3 inhibitors of the invention, so long as it includes the required sequence and is able to function as the peptide of the invention as described herein. It is noted that both shorter active fragments derived from the inhibitors denoted as SEQ ID NOS: 1-49, 113, 114, and longer polypeptides or peptides comprising these sequences are within the scope of the present invention.

Whenever NS3 inhibitors are mentioned in the invention, also salts and functional derivatives thereof are contemplated, as long as the biological activity of the peptide with respect to HCV is maintained. The present invention encompasses any analog, derivative, and conjugate containing the NS3 inhibitors of the invention, so long as the peptide is capable of inhibiting NS3 activity. Thus, the present invention encompasses polypeptides or peptides containing non-natural amino acid derivatives or non-protein side chains.

The term “analog” includes any polypeptide or peptide having an amino acid sequence substantially identical to one of the sequences specifically shown herein in which one or more residues have been conservatively substituted with a functionally similar residue and which displays the abilities as described herein. Examples of conservative substitutions include the substitution of one non-polar (hydrophobic) residue such as isoleucine, valine, leucine or methionine for another, the substitution of one polar (hydrophilic) residue for another such as between arginine and lysine, between glutamine and asparagine, between glycine and serine, the substitution of one basic residue such as lysine, arginine or histidine for another, or the substitution of one acidic residue, such as aspartic acid or glutamic acid for another.

The phrase “conservative substitution” also includes the use of a chemically derivatized residue in place of a non-derivatized residue provided that such peptide displays the requisite inhibitory function on HCV as specified herein.

The term derivative includes any chemical derivative of the polypeptides or peptides of the invention having one or more residues chemically derivatized by reaction of side chains or functional groups. Such derivatized molecules include, for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups may be derivatized to form salts, methyl and ethyl esters or other types of esters or hydrazides. Free hydroxyl groups may be derivatized to form O-acyl or O-alkyl derivatives. The imidazole nitrogen of histidine may be derivatized to form N-im-benzylhistidine. Also included, as chemical derivatives are those peptides, which contain one or more naturally occurring amino acid derivatives of the twenty standard amino acid residues. For example: 4-hydroxyproline may be substituted for proline; 5-hydroxylysine may be substituted for lysine; 3-methylhistidine may be substituted for histidine; homoserine may be substituted or serine; and ornithine may be substituted for lysine.

In addition, a polypeptide or peptide derivative can differ from the natural sequence of the polypeptides or peptides of the invention by chemical modifications including, but are not limited to, terminal-NHb acylation, acetylation, or thioglycolic acid amidation, and by terminal-carboxlyamidation, e.g., with ammonia, methylamine, and the like. Peptides can be either linear, cyclic or branched and the like, which conformations can be achieved using methods well-known in the art.

Polypeptides or peptides of the present invention also include any polypeptide or peptide having one or more additions and/or deletions of residues relative to the sequence of the fusion peptide of the invention, so long as the requisite inhibitory activity is maintained.

Addition of amino acid residues may be performed at either terminus of the polypeptides or peptides of the invention for the purpose of providing a “linker” by which the peptides of this invention can be conveniently bound to a carrier. Such linkers are usually of at least one amino acid residue and can be of 40 or more residues, more often of 1 to 10 residues. Typical amino acid residues used for linking are serine, glycine, tyrosine, cysteine, lysine, glutamic and aspartic acid, or the like.

A polypeptide or peptide of the present invention may be coupled to or conjugated with another protein or polypeptide to produce a conjugate. Such a conjugate may have advantages over the polypeptide or peptide used alone. By means of a non-limitative example, NS3 inhibitors of the invention may be conjugated to a sequence facilitating internalization of the inhibitors into cells, including, but not limited to, protein-transduction domain (PTD, see e.g. Beerens et al., 2003).

In other embodiments, the NS3 inhibitors of the invention are associated with other internalization moieties, such as compounds, liposomes or particles which improve the ability of the NS3 inhibitors to penetrate the lipid bilayer of the cellular plasma membrane and enter the cytoplasm. As used herein, the term “associated with” refers to covalent attachment or a non-covalent interaction mediated by, for example, ionic bonds, hydrogen bonds, van der waals forces and/or hydrophobic interactions, such that the internalization moiety and NS3 inhibitor remain in close proximity under physiological conditions. Various particle-, liposome- and ligand-mediated delivery systems are available, and their use is well known to those of ordinary skill in the art.

Pharmaceutical Compositions

The NS3 inhibitors of the present invention can be administered to an organism per se, or in a pharmaceutical composition where it is mixed with suitable carriers or excipients. The purpose of a pharmaceutical composition is to facilitate administration of a compound to an organism.

Hereinafter, the phrases “physiologically acceptable carrier” and “pharmaceutically acceptable carrier”, which may be used interchangeably, refer to a carrier or a diluent that does not cause significant irritation to an organism and does not abrogate the biological activity and properties of the administered compound. Useful carriers include, for example, water, acetone, ethanol, ethylene glycol, propylene glycol, butane-1,3-diol, isopropyl myristate, isopropyl palmitate, or mineral oil.

Herein, the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils, and polyethylene glycols.

The NS3 inhibitors of the invention may be formulated into the pharmaceutical composition as a neutralized pharmaceutically acceptable salt form. Pharmaceutically acceptable salts include the acid addition salts (formed with the free amino groups of the polypeptide), which are formed with inorganic acids, such as for example, hydrochloric or phosphoric acid, or with organic acids such as acetic, oxalic, tartaric, and the like.

Suitable bases capable of forming salts with the polypeptides or peptides of the present invention include, but are not limited to, inorganic bases such as sodium hydroxide, ammonium hydroxide, potassium hydroxide and the like; and organic bases such as mono-, di- and tri-alkyl and aryl amines (e.g. triethylamine, diisopropyl amine, methyl amine, dimethyl amine and the like) and optionally substituted ethanolamines (e.g. ethanolamine, diethanolamine and the like).

Techniques for formulation and administration of drugs may be found in the latest edition of “Remington's Pharmaceutical Sciences,” Mack Publishing Co., Easton, Pa., which is herein fully incorporated by reference.

Suitable routes of administration may, for example, include oral, rectal, transmucosal, especially transnasal, intestinal, or parenteral delivery, including intramuscular, subcutaneous, and intramedullary injections, as well as intrathecal, direct intraventricular, intravenous, intraperitoneal or intranasal injections. Alternately, one may administer the pharmaceutical composition in a local rather than systemic manner, for example, via injection of the pharmaceutical composition directly into a tissue region of a patient.

Apart from other considerations, the administration of peptides, peptide analogs or vectors or cells dictates that the formulation be suitable for delivery of these type of compounds. Clearly, peptides are less suitable for oral administration due to susceptibility to digestion by gastric acids or intestinal enzymes. It is contemplated that the present invention encompasses peptide compositions designed to circumvent these problems. The preferred routes of administration of peptides are intra-articular, intravenous, intramuscular, subcutaneous, intradermal, or intrathecal. A more preferred route is by direct injection at or near the site of disorder or disease.

Pharmaceutical compositions of the present invention may be manufactured by processes well known in the art, e.g., by means of conventional mixing, dissolving, granulating, dragee-making, levigating, emulsifying, encapsulating, entrapping, or lyophilizing processes.

Pharmaceutical compositions for use in accordance with the present invention thus may be formulated in conventional manner using one or more physiologically acceptable carriers comprising excipients and auxiliaries, which facilitate processing of the active ingredients into preparations that can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries as desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, and sodium carbomethylcellulose; and/or physiologically acceptable polymers such as polyvinylpyrrolidone (PVP). If desired, disintegrating agents, such as cross-linked polyvinyl pyrrolidone, agar, or alginic acid or a salt thereof, such as sodium alginate, may be added.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions, and suitable organic solvents or solvent mixtures. Dyestuffs or pigments may be added to the tablets or dragee coatings for identification or to characterize different combinations of active compound doses.

Pharmaceutical compositions that can be used orally include push-fit capsules made of gelatin, as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate, and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane, or carbon dioxide. In the case of a pressurized aerosol, the dosage may be determined by providing a valve to deliver a metered amount. Capsules and cartridges of, for example, gelatin for use in a dispenser may be formulated containing a powder mix of the compound and a suitable powder base, such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with, optionally, an added preservative. The compositions may be suspensions, solutions, or emulsions in oily or aqueous vehicles, and may contain formulatory agents such as suspending, stabilizing, and/or dispersing agents.

Pharmaceutical compositions for parenteral administration include aqueous solutions of the active preparation in water-soluble form. Additionally, suspensions of the active ingredients may be prepared as appropriate oily or water-based injection suspensions. Suitable lipophilic solvents or vehicles include fatty oils such as sesame oil, or synthetic fatty acid esters such as ethyl oleate, triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the active ingredients, to allow for the preparation of highly concentrated solutions.

Alternatively, the active ingredient may be in powder form for constitution with a suitable vehicle, e.g., a sterile, pyrogen-free, water-based solution, before use.

The pharmaceutical composition of the present invention may also be formulated in rectal compositions such as suppositories or retention enemas, using, for example, conventional suppository bases such as cocoa butter or other glycerides.

Pharmaceutical compositions suitable for use in the context of the present invention include compositions wherein the active ingredients are contained in an amount effective to achieve the intended purpose. More specifically, a “therapeutically effective amount” means an amount of active ingredients (e.g., a nucleic acid construct) effective to prevent, alleviate, or ameliorate symptoms of a disorder (e.g., ischemia) or prolong the survival of the subject being treated.

Determination of a therapeutically effective amount is well within the capability of those skilled in the art, especially in light of the detailed disclosure provided herein.

For any preparation used in the methods of the invention, the dosage or the therapeutically effective amount can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration, and dosage can be chosen by the individual physician in view of the patient's condition. (See, e.g., Fingl, E. et al. (1975), “The Pharmacological Basis of Therapeutics”, Ch. 1, p. 1.)

Dosage amount and administration intervals may be adjusted individually to provide sufficient plasma or brain levels of the active ingredient to induce or suppress the biological effect (i.e., minimally effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks, or until cure is effected or diminution of the disease state is achieved.

The amount of a composition to be administered will, of course, be dependent on the subject being treated, the severity of the affliction, the manner of administration, the judgment of the prescribing physician, etc.

Compositions of the present invention may, if desired, be presented in a pack or dispenser device, such as an FDA-approved kit, which may contain one or more unit dosage forms containing the active ingredient. The pack may, for example, comprise metal or plastic foil, such as a blister pack. The pack or dispenser device may be accompanied by instructions for administration. The pack or dispenser device may also be accompanied by a notice in a form prescribed by a governmental agency regulating the manufacture, use, or sale of pharmaceuticals, which notice is reflective of approval by the agency of, the form of the compositions for human or veterinary administration. Such notice, for example, may include labeling approved by the U.S. Food and Drug Administration for prescription drugs or of an approved product insert. Compositions comprising a preparation of the invention formulated in a pharmaceutically acceptable carrier may also be prepared, placed in an appropriate container, and labeled for treatment of an indicated condition, as further detailed above.

For the use of nucleic acid based pharmaceutical compositions, preferred carriers are capable of maintaining a nucleic acid molecule of the present invention in a form that, upon arrival of the nucleic acid molecule to a cell, the nucleic acid molecule is capable of entering the cell and being expressed by the cell. Carriers of the present invention include: (1) excipients or formularies that transport, but do not specifically target a nucleic acid molecule to a cell (referred to herein as non-targeting carriers); and (2) excipients or formularies that deliver a nucleic acid molecule to a specific site in an animal or a specific cell (i.e., targeting carriers). Examples of non-targeting carriers include, but are not limited to water; phosphate buffered saline, Ringer's solution, dextrose solution, serum-containing solutions, Hank's solution, other aqueous physiologically balanced solutions, oils, esters and glycols. Aqueous carriers can contain suitable auxiliary substances required to approximate the physiological conditions of the recipient, for example, by enhancing chemical stability and isotonicity.

Suitable auxiliary substances include, for example, sodium acetate, sodium chloride, sodium lactate, potassium chloride, calcium chloride, and other substances used to produce phosphate buffer, Tris buffer, and bicarbonate buffer. Auxiliary substances can also include preservatives, such as thimerosal, m- and o-cresol, formalin and benzol alcohol. Preferred auxiliary substances for aerosol delivery include surfactant substances non-toxic to an animal, for example, esters or partial esters of fatty acids containing from about six to about twenty-two carbon atoms. Examples of esters include, caproic, octanoic, lauric, palmitic, stearic, linoleic, linolenic, olesteric, and oleic acids. Other carriers can include metal particles (e.g., gold particles) for use with, for example, a biolistic gun through the skin. Pharmaceutical compositions of the present invention can be sterilized by conventional methods.

Targeting carriers are herein referred to as “delivery vehicles”. Delivery vehicles of the present invention are capable of delivering a pharmaceutical composition of the present invention to a target site in an animal. A “target site” refers to a site in an animal to which one desires to deliver a pharmaceutical composition. Examples of delivery vehicles include, but are not limited to, artificial and natural lipid-containing delivery vehicles. Natural lipid-containing delivery vehicles include cells and cellular membranes. Artificial lipid-containing delivery vehicles include liposomes and micelles.

A delivery vehicle of the present invention can be modified to target to a particular site in an animal, thereby targeting and making use of a nucleic acid molecule of the present invention at that site. Suitable modifications include manipulating the chemical formula of the lipid portion of the delivery vehicle and/or introducing into the vehicle a compound capable of specifically targeting a delivery vehicle to a preferred site, for example, a preferred cell type. Specifically targeting refers to causing a delivery vehicle to bind to a particular cell by the interaction of the compound in the vehicle to a molecule on the surface of the cell. Suitable targeting compounds include ligands capable of selectively (i.e., specifically) binding another molecule at a particular site. Examples of such ligands include antibodies, antigens, receptors and receptor ligands. Manipulating the chemical formula of the lipid portion of the delivery vehicle can modulate the extracellular or intracellular targeting of the delivery vehicle. For example, a chemical can be added to the lipid formula of a liposome that alters the charge of the lipid bilayer of the liposome so that the liposome fuses with particular cells having particular charge characteristics.

According to another embodiment, the delivery vehicle of the present invention may be a liposome. A liposome is capable of remaining stable in an animal for a sufficient amount of time to deliver a nucleic acid sequence of the present invention to a preferred site in the animal. A liposome of the present invention is preferably stable in the animal into which it has been administered for at least about 30 minutes, more preferably for at least about 1 hour and even more preferably for at least about 24 hours.

Suitable liposomes for use with the present invention include any liposome. Preferred liposomes of the present invention include those liposomes standardly used in, for example, gene delivery methods known to those of skill in the art. More preferred liposomes comprise liposomes having a polycationic lipid composition and/or liposomes having a cholesterol backbone conjugated to polyethylene glycol.

Therapeutic Use

In another aspect, the invention provides a method of treating HCV infection in a subject in need thereof, comprising administering to the subject a therapeutically effective amount of a pharmaceutical composition comprising an NS3 inhibitor of the invention.

In another aspect, the invention provides a method of treating or preventing the symptoms of hepatitis in a subject in need thereof, comprising administering to the subject a therapeutically effective amount of a pharmaceutical composition comprising an NS3 inhibitor of the invention.

In another aspect, the invention provides a method of preventing liver damage in a subject in need thereof, comprising administering to the subject a therapeutically effective amount of a pharmaceutical composition comprising an NS3 inhibitor of the invention.

In another aspect, the invention provides a method of preventing HCC in a subject in need thereof, comprising administering to the subject a therapeutically effective amount of a pharmaceutical composition comprising an NS3 inhibitor of the invention.

In another aspect, the invention provides a method of treating HCV infection in a subject in need thereof, comprising administering to the subject a therapeutically effective amount of a pharmaceutical composition comprising a recombinant construct comprising an isolated nucleic acid sequence encoding an NS3 inhibitor of the invention operably linked to one or more transcription control elements, thereby treating HCV infection.

In another aspect, the invention provides a method of treating or preventing the symptoms of hepatitis in a subject in need thereof, comprising administering to the subject a therapeutically effective amount of a pharmaceutical composition comprising a recombinant construct comprising an isolated nucleic acid sequence encoding an NS3 inhibitor of the invention operably linked to one or more transcription control elements, thereby treating or preventing the symptoms of hepatitis.

In another aspect, the invention provides a method of preventing liver damage in a subject in need thereof, comprising administering to the subject a therapeutically effective amount of a pharmaceutical composition comprising a recombinant construct comprising an isolated nucleic acid sequence encoding an NS3 inhibitor of the invention operably linked to one or more transcription control elements, thereby preventing liver damage.

In another aspect, the invention provides a method of preventing HCC in a subject in need thereof, comprising administering to the subject a therapeutically effective amount of a pharmaceutical composition comprising a recombinant construct comprising an isolated nucleic acid sequence encoding an NS3 inhibitor of the invention operably linked to one or more transcription control elements, thereby preventing HCC.

In another aspect, the invention provides a method of treating HCV infection in a subject in need thereof, comprising: a) obtaining cells from the subject; b) contacting the cells ex vivo with a therapeutically effective amount of a pharmaceutical composition comprising a recombinant construct comprising an isolated nucleic acid sequence encoding NS3 inhibitor of the invention operably linked to one or more transcription control elements; and c) re-introducing said cells to said subject, thereby treating HCV infection in said subject.

In another aspect, the invention provides a method of treating or preventing the symptoms of hepatitis in a subject in need thereof, comprising: a) obtaining cells from the subject; b) contacting the cells ex vivo with a therapeutically effective amount of a pharmaceutical composition comprising a recombinant construct comprising an isolated nucleic acid sequence encoding NS3 inhibitor of the invention operably linked to one or more transcription control elements; and c) re-introducing said cells to said subject, thereby treating or preventing the symptoms of hepatitis.

In another aspect, the invention provides a method of preventing liver damage in a subject in need thereof, comprising: a) obtaining cells from the subject; b) contacting the cells ex vivo with a therapeutically effective amount of a pharmaceutical composition comprising a recombinant construct comprising an isolated nucleic acid sequence encoding NS3 inhibitor of the invention operably linked to one or more transcription control elements; and c) re-introducing said cells to said subject, thereby preventing liver damage in said subject.

In another aspect, the invention provides a method of preventing HCC in a subject in need thereof, comprising: a) obtaining cells from the subject; b) contacting the cells ex vivo with a therapeutically effective amount of a pharmaceutical composition comprising a recombinant construct comprising an isolated nucleic acid sequence encoding NS3 inhibitor of the invention operably linked to one or more transcription control elements; and c) re-introducing said cells to said subject, thereby preventing HCC in said subject.

In another aspect, the invention provides a method of preventing HCV infection in a liver transplant, comprising: a) treating a liver transplant before transplantation ex vivo with a therapeutically effective amount of a pharmaceutical composition comprising a recombinant construct comprising an isolated nucleic acid sequence encoding NS3 inhibitor of the invention operably linked to one or more transcription control elements; and b) transplanting the liver transplant to a subject in need thereof, thereby generating an HCV-immune liver transplant.

The NS3 inhibitors of the invention may be used alone or in combination with one or more therapeutic agents, including, but not limited to: interferon (pegylated or not), ribavirin, or one or more other anti-HCV agents, all of which administered together or separately, e.g., prior to, concurrently with or following the administration of other NS3 inhibitors of the invention or pharmaceutically acceptable salts thereof.

In order to treat a subject with a disease, a pharmaceutical composition of the resent invention is administered to the subject in an effective manner such that the composition is capable of treating that subject from disease. According to the present invention, treatment of a disease refers to alleviating a disease and/or preventing the development of a secondary disease resulting from the occurrence of a primary disease. An effective administration protocol (i.e., administering a pharmaceutical composition in an effective manner) comprises suitable dose parameters and modes of administration that result in treatment of a disease. Effective dose parameters and modes of administration can be determined using methods standard in the art for a particular disease. Such methods include, for example, determination of survival rates, side effects (i.e., toxicity) and progression or regression of disease.

In accordance with the present invention, a suitable single dose size is a dose that is capable of treating a subject with disease when administered one or more times over a suitable time period. Doses of a pharmaceutical composition of the present invention suitable for use with direct injection techniques can be used by one of skill in the art to determine appropriate single dose sizes for systemic administration based on the size of a subject.

The following examples are presented in order to more fully illustrate some embodiments of the invention. They should, in no way be construed, however, as limiting the broad scope of the invention.

### EXAMPLES

### Materials and Methods

Construction of a reporter plasmid for insertion of peptide coding sequences into β-galactosidase. Plasmid pIB13 (Benhar and Engelberg-Kulka 1993) carries the 5′ 77 codons of the E. coli trpR gene fused to the 8^{th} codon of the E. coli lacZ gene, under the control of the trpR promoter on a pBR322 plasmid backbone (coIEl replicon). pIB13 DNA was digested with the restriction enzymes CIaI and Dral and a 4089 bp DNA fragment was recovered. A p15A replicon and kanamycin resistance cassette carrying 3054 bp DNA fragment was isolated by Dral and BstBl digestion of pACYC177 plasmid DNA. The two DNA fragments were ligated and used to transform of E. coli TG-I cells that resulted in plasmid pEB13.

Two tandem sfll restriction sites separated by a TAA stop codon were inserted between lacZ codons 279-280 by overlap-extension PCR-based mutagenesis (Berdichevsky, Zemel et al., 2003) as follows: pEB13 DNA was used as template in two PCR reactions using primers LacZ-Sfi-279-280-BACK (GAAATTATCGATGAGGGCCAACGTGGCCGGTAATTTTTGGCCTCTGGGGCCCGTGGTGGTTATGCC, SEQ ID NO:71) with LacZ-520-FOR (CAGCCGGGAAGGGCTGGTCTT, SEQ ID NO:72), and LacZ-70-BACK (CCGGAAAGCTGGCTGGAGTGC, SEQ ID NO:73) with LacZ-279-FOR (GGCCCTCATCGATAATTTCAC, SEQ ID NO:74). All the PCR reactions were carried out using 30 cycles of 95° C. for 30 sec, 55° C. for 1 min and 72° C. for 1 min with a final extension at 72° C. for 5 min. The resulting PCR products of 667 bp and 763 bp, respectively, were mixed and assembled by PCR amplification using primers LacZ-70-BACK and LacZ-520-FOR. The resulting PCR product of 1390 bp was digested with Bsui βl and BssEll and cloned into vector DNA recovered by digesting pEB13 DNA with the same enzymes. Ligated DNA was used to transform DH5α E. coli cells (Invitrogen, USA) that were plated on LB agar plates supplemented with 0.04% (w/v) X-gal. White (colorless) colonies that resulted from interruption of the lacZ open reading frame by the inserted stop codon were analyzed by DNA sequencing. The resulting plasmid was pEB13-Sfi (SEQ ID NO:82). This plasmid serves as a universal platform for insertion of protease-site-coding sequences between lacZ codons 279-280, as illustrated below for the NS5A/B cleavage site of NS3.

Insertion of the NS3-cleavable NS5A/B site into β-galactosidase. The NS5A/B cleavage site of NS3 was engineered between codons 279-280 of the lacZ gene on plasmid pEB13-Sfi as follows: A DNA duplex formed by annealing primers NS5A/B-SE (AGCTAGCGAGGACGTCGTCTGCTGCTCGATGTCCTACACTG, SEQ ID NO:75) and NS5A/B-AS (TGTAGGACATCGAGCAGCAGACGACGTCCTCGCTAGCTCGT, SEQ ID NO:76) was inserted into pEB13-Sfi plasmid DNA that had been digested with Sfll. Ligated DNA was used to transform TG-I E. coli cells (Amersham Biosciences, USA) that were plated on X-gal supplemented LB agar plates. Blue colonies that resulted from restoration of the lacZ open reading frame by the inserted fragment were analyzed by DNA sequencing. The resulting plasmid was pEB13-/acZ_{NS5}A_{/}B (SEQ ID NO:68).

FIG. 5 illustrates the amino acid sequence of the resulting reporter protein, wherein the 78 N-terminal residues of the E. coli frpR gene product followed by a short linker are presented in italics, and the NS3-cleavable NS5A/B site is presented in bold type. The complete amino acid sequence of the reporter protein is denoted as SEQ ID NO:64; the amino acid sequence of the NS5A/B-containing β-galactosidase, without the preceding sequence corresponding to the trpR and linker, is denoted as SEQ ID NO: 65.

Cloning of NS3 under the araBAD promoter. Single-chain NS3 protease (,scNS3), which is a single-chain fusion linking the NS4A cofactor peptide to the N-terminus of the NS3 protease (Berdichevsky et al., 2003), was stabilized by its expression as a maltose-binding protein fusion (Bach et al., 2001) and cloned under the control of the araBAD promoter. FIG. 6 illustrates the amino-acid sequence of the engineered enzyme (denotes as SEQ ID NO: 66): a fusion protein comprised of the E. coli malE gene product (MBP) fused the NS4A peptide (in bold type) followed by the NS3 protease domain derived, HCV genotype Ib (Berdichevsky et al., 2003). The cloning was preformed as follows: Plasmid pYB43 (Berdichevsky et al., 2003) was used as template in a PCR reaction with primers NS3-Nco-BACK (TCAGTACCATGGCGCCTATCGGCTCAGTAGTA, SEQ ID NO:77) and NS3-Not-FOR (GGGAAAGCGGCCGCTTACCGCATAGTGGTTTCCATAGA, SEQ ID NO:78). The resulting PCR product of 637 bp was digested with Ncol and Notl and cloned into 6128 bp vector DNA fragment recovered by digesting pMALc-NN DNA (Bach et al., 2001) with the same enzymes. Ligated DNA was used to transform TG-I E. coli cells. The resulting plasmid was pMALC-NN-scNSS.

Next, the MBP-ˆcNS3 carrying fragment was recovered by PCR using pMALC-NN-scNS3 as template with primers MalE-B spHI-BACK (CCCTAATCATGAAAACTGAAGAAGGTAAA, SEQ ID NO:79) and NS3-Not-FOR. The resulting 1788 bp PCR product was digested with BspHI and iVbtl and cloned into a 3790 bp vector DNA fragment recovered by digesting pBAD-TOPO DNA (Invitrogen, USA) with Ncol and Nod. Ligated DNA was used to transform TG-I E. coli cells. The resulting plasmid was named pBAD-MBP-ˆcNS3. A similar plasmid, pBAD-MBP-scNS3ₘᵤₜ was constructed for expression of an inactive mutant S-1 165-A NS3 (Zemel et al., 2001) to serve as a negative control.

Next, a bi-cistronic plasmid was constructed, carrying both lacZ with the inserted NS3 cleavage site (lacZ-ˆss_{A}m) and MBP-,scNS3 as follows: Restriction sites for Sphl and NotI were inserted into vector pEB13/αcZ_{N}_{_{S5A/B}} as a DNA duplex formed by annealing primers pEB13-dup-SE (AATTGCATGCCTGCAGGCGGCCGCG, SEQ ID NO:80) and pEB13-dup-AS (AATTCGCGGCCGCCTGCAGGCATGC, SEQ ID NO:81). The duplex was inserted into pEB13-føcZ.Ns₅A_{/}B plasmid DNA that had been linearized at the unique EcoRI site. The resulting plasmid was digested with Sphl and Notl to obtain a vector fragment of 7822 bp. An insert fragment of 3074 bp corresponding to MBP-scNS3 with its controlling araBAD promoter and the regulatory araC gene was obtained by digesting plasmid pBAD-MBP-,scNS3 with Sphl and Notl. The two fragments were ligated to generate plasmid pMGT14 (SEQ ID NO: 67). A similar plasmid, pMGT15, expressing an inactive mutant S-1165-A NS3 (Zemel et al., 2001) was constructed using the Sphl-Notl fragment of plasmid pBAD-MBP-ˆcNS3ₘᵤₜ to serve as a negative control of catalysis. In both plasmids the expression of the engineered lacZ gene is weakly constitutive in trpR⁺ E. coli strains while MBP-scNS3 is inducible by arabinose.

Construction of a murine immune antibody phage display library. MBP-scNS3 protein was expressed and purified essentially as described for MBP-scFvs (Bach et al., 2001). Balb/c mice were immunized with 50 μg of purified MBP-,scNS3 in complete Freunds adjuvant and boosted three times at two-week intervals using 50 μg of purified MBP-ˆcNS3 in incomplete Freunds adjuvant. The mice were bled three days after each boost. The serum anti-NS3 antibody titer was determined by ELISA.

Total RNA was extracted from 10⁷ cells recovered from the immune mice spleens according to the instructions of the Tri Reagent total RNA extraction kit (Talron, Israel). A total of 250 μg RNA were obtained. cDNA was produced from mRNA by reverse transcription reactions using 1 μg of total RNA as template. Heavy and light chain variable domains were amplified in PCR using a primer set designed for the construction of mouse scFv libraries (Benhar and Reiter 2002). The antibody variable cassettes were combinatorially assembled into complete scFv constructs and cloned into pCC phagemid vector (Berdichevsky et al., 1999) essentially as described (Benhar and Reiter 2002). The cloned vectors were transferred into competent E. coli TG-I cells by electroporation and library diversity was tested as described (Benhar and Reiter 2002).

Applying the bacterial genetic screening to isolate NS3-inhibitory scFvs. Affinity selected phages were pooled and used as source for scFv DNA by digestion of phagemid DNA with Ncol and Notl. The pooled insert DNA was cloned into pMALc-NN that had been digested with the same enzymes essentially as described (Bach et al., 2001). In pMALc-NN, expression of MBP-scFvs is under the control of an IPTG-inducible tac promoter and the plasmid carries an ampicillin resistance cassette and the colEl origin of replication and is thus compatible with pMGT14 or pMGT15. The plasmid pool was introduced into E. coli TG-I cells already containing the pMGT14 plasmid. The transformants were plated to yield individual colonies on 2xYT agar plates supplemented with 0.004% (w/v) X-gal, 100 μg/ml ampicillin, 50 μg/ml kanamycin, 0.2% arabinose and 0.05 mM IPTG. Blue colonies were picked and pMALc-NN-scFv DNA was recovered and re-introduced into pMGT14 expressing cells to validate the results of the initial screening.

Binding assays. Selected MBP-scFvs were expressed and purified from the soluble fraction of IPTG-induced plasmid-carrying E. Coli BL21 cells using amylose resin chromatography (Bach et al., 2001). ScNS3 was overexpressed in pYB-43 carrying E. Coli BL-21 (DE3) cells by a modification of the inventors' published method (Berdichevsky et al., 2003). The bacteria were induced with 1 mM IPTG at 37° C. for 3 hr, which resulted in accumulation of the scNS3 as inclusion bodies. The inclusion bodies were solubilized in 6M guanidinium hydrochloride and purified by Talon (Clontech, USA) IMAC chromatography under denaturing conditions. The purified protein was stored at −80° C. ELISA plates were coated with 4 μg/ml ,scNS3 in NaHCO₃ buffer pH 9.6. ELISAs were processed and developed as described (Benhar and Reiter 2002) using a mouse anti-MBP antibody (Sigma, Israel) followed by HRP conjugated goat anti mouse antibodies (Jackson Immunolaboratories, USA).

For competition ELISA the inventors replaced the C-terminal FLAG epitope of the scFvs that were used as tracers with a myc tag. Competing scFvs were added to scNS3-coated plates at varying concentrations and incubated at room temperature for 1 hr before adding the tracer scFvs. Binding of the tracers was monitored using a HRP-conjugated anti myc antibody (Sigma, Israel).

NS3 catalysis inhibition assays. Blue colonies of E. coli TG-I cells carrying pMGT14 and pMALc-NN-scFv that were isolated by the genetic screening were grown in LB medium supplemented with 100 μg/ml ampicillin, 50 μg/ml kanamycin and induced with 0.2% (w/v) arabinose and 0.5 mM IPTG at O.D₆₀₀ₙₘ=0.6 for 4 hr at 30° C. To measure the β-galactosidase activity an ONPG hydrolysis assay (Bach et al., 2001) was carried out. An in vitro fluorometric assay for the measurement of NS3 protease catalysis inhibition by the purified scFvs was carried out essentially as described (Berdichevsky et al., 2003) with the following modifications: the EFGP-NS5A/B-CBD substrate was immobilized onto cellulose prior to its exposure to enzyme and inhibitor. The reactions were carried out in 96 well plates in a volume of 100 μl containing 5 μM immobilized substrate, 100 nM MBP-5cNS3 and 2.4 or 1.2 μM of tested MBP-scFv.

Immunoblot. Was carried out to follow the time course on NS3 induction and its effect on cleavage of lacZNS5A/B gene product. TG-I cells carrying plasmids pMGT14 or pMGT15 were grown in LB medium supplemented with 100 μg/ml ampicillin, 50 μg/ml kanamycin to an O.D₆₀₀ₙₘ=0.6. The cells were than induced with 0.2% (w/v) arabinose at 30° C. 20 μg of total cell extracts were separated by SDS/PAGE and transferred onto nitrocellulose membranes. The membranes were reacted with mouse polyclonal anti β-galactosidase antiserum (Lab collection) and a mouse anti-MBP antibody and detected by HRP conjugated goat anti mouse antibody (Jackson Immunolaboratories, USA). The membrane was developed by using ECL reagents (Berdichevsky et al., 2003).

Cells and transfections. HCV genotype Ib strain N subgenomic replicon cell lines were obtained from Stanley Lemon and are described in (Bourne et al., 2005). En5-3 cells are a clonal cell line derived from Huh7 cells by stable transformation with the plasmid pLTR-SEAP. These cells were cultured in DMEM supplemented with 10% fetal calf serum, 2 μg/ml blasticidin (Invitrogen), penicillin, and streptomycin. Following transfection with Ntat2ANeo replicon RNAs, cells supporting replicon amplification were selected and maintained in the above media containing in addition 400 μg/ml G418 (geneticin). Cell lines were passaged once or twice per week. For inhibition assay, replicon cell were transfected with NS3-neutralizing scFvs using the FuGENE reagent. The culture medium was replaced every 24 h post transfection and the secreted alkaline phosphatase (SEAP) activity was measured in these fluids as described below, reflecting the daily production of SEAP by the cells.

Alkaline phosphatase assay. SEAP activity was measured in supernatant culture fluids using the Phospha-Light Chemiluminescent Reporter Assay (Tropix) with the manufacturer's suggested protocol reduced 1/3 in scale as following: a 10 μl aliquots of the supernatant fluids from transfected cells were mixed in a tube with 30 μl of 1× diluted 5× dilution buffer in sterile water. The tubes were heated to 65° C. for 30 min. 35 μl of the samples were transferred to Luminometer tubes and 35 μl of assay buffer were added to the tubes and incubated for 5 min at room temperature. 35 μl of CSPD substrate diluted 1/20 in reaction buffer was added to the tubes and the tubes were incubated for 20 min at room temperature. The luminescent signal was read using a TD-20/20 Luminometer (Turner Designs, Inc.).

### Example 1

### A Bacterial Genetic Screening for the Isolation of NS3 Protease Inhibitors

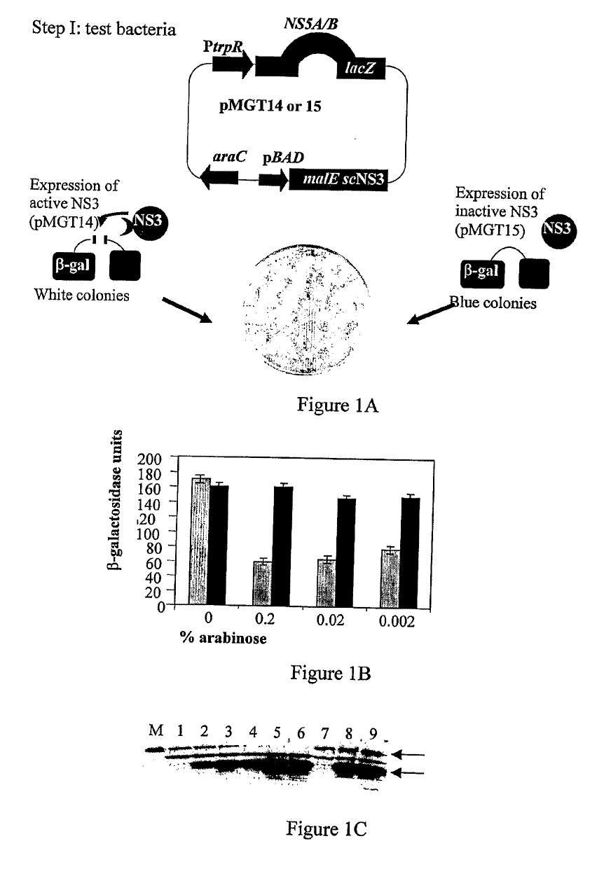

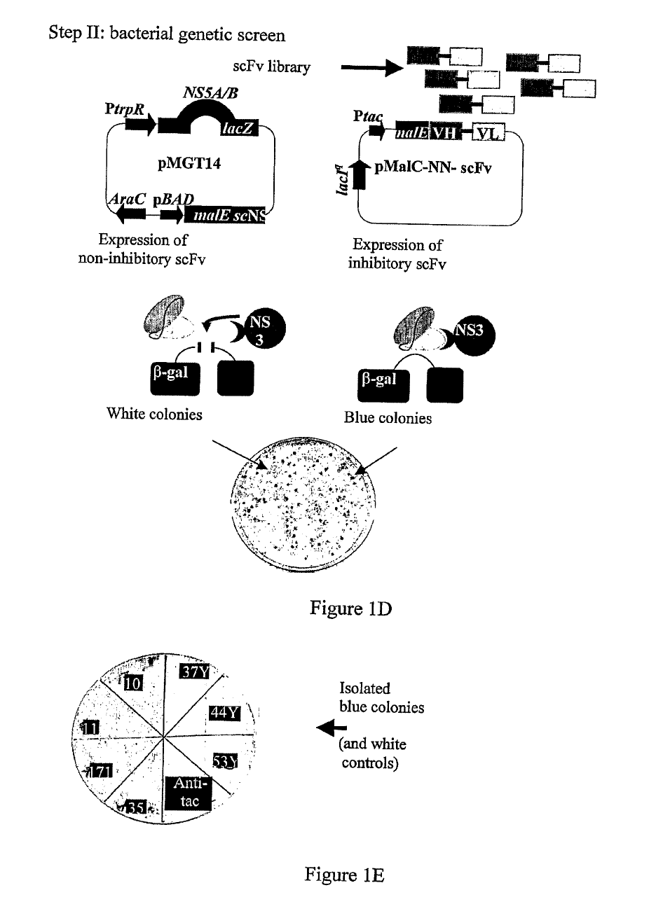

A novel bacterial genetic screening was established, based on phenotypic changes that result from the concerted co-expression of enzyme, substrate and potential inhibitor, to identify NS3-neutralizing antibodies directly from a large pool of clones. Vector pMGT14 (SEQ ID NO:67, FIG. 1A) was designed to allow the expression of the enzyme, MBP-ˆcNS3 (SEQ ID NO:66) and the substrate (SEQ ID NO:64), an engineered β-galactosidase, which is cleaved by NS3 at the NS5A/B site. Co-expression of an inhibitor in pMGT14 carrying E. coli cells results in the phenotypic changes that make it possible to identify and characterize such NS3 inhibitors.

The genetic screening is based on lacZ as a reporter gene that yields blue colonies when expressed in E. coli cells that are plated on X-gal indicative plates. The engineered lacZ gene in the disclosed system was designed to be constitutively expressed at a low level. Thus, the trpR promoter controlled trpR-lacZ fusion (Benhar and Engelberg-Kulka 1993) was chosen. The reporter gene was transferred onto a low copy number p15A replicon plasmid that also carries a kanamycin resistance gene to make it compatible with subsequently introduced inhibitor-coding plasmids that are base on colEl replicon, high copy number, ampicillin resistance-carrying plasmids.

Initially, two tandem sfil site separated by a TAA stop codon were inserted between codons 279-280 of the lacZ gene. The insertion site for enzyme cleavage sequences in lacZ was chosen based on initial studies of engineered β-galactosidase as a biosensor for antibody binding (Feliu et al., 2002). This resulted in plasmid pEB13-Sfi (SEQ ID NO:82) that does not encode a functional β-galactosidase because its open reading frame is interrupted by the inserted stop codon. Plasmid pEB13-Sfi may serve as a general purpose substrate plasmid by the insertion of any desirable sequence that specifies a protease cleavage site as a staggered DNA duplex. Such insertion restores the lacZ open reading frame, allowing the identification of the cleavage site engineered lacZ carrying plasmids as blue colonies upon plating transformed E. coli on X-gal plates. A sequence specifying the NS5A/B site was inserted, yielding an NS3-cleavable β-galactosidase enzyme.

To test the activity of the NS5A/B site-containing β-galactosidase encoded by vector pEB13-lacZNS5A/B, an ONPG hydrolysis assay (Bach et al., 2001) was applied. The engineered enzyme had 12% β-galactosidase enzymatic activity in comparison to the wild type lacZ carried on plasmid pEB13 (Table 1). Still, cells expressing it formed blue colonies on X-gal indicative plates.

In previous studies, the inventors expressed scNS3 in bacteria and purified it in an active form (Berdichevsky et al., 2003; Zemel et al., 2004). The inventors found that the MBP fusion technology developed for efficient cytoplasmic expression of scFvs (Bach et al., 2001) could be adopted to improve the expression of ,scNS3 without compromising its activity. Thus, the current study was carried out using MBP-/cNS3. To test the ability of the engineered lacZ_{N}ss_{A/}B to detect NS3 protease activity in vivo, pMALC-NN-ˆcNS3 was co-introduced with pEB13-lacZNssA/B into E. coli TG-I cells. As a control, pMALc-NN-EGFP that codes for enhanced green fluorescent protein (Bach et al., 2001) was used. As shown in Table 1, induction of scNS3 expression with IPTG resulted in reduced β-galactosidase activity in comparison to the EGFP control. In addition, TG-I cells carrying pMALC-NN-scNS3 and pEB13-lacZNs₅A/B formed white colonies on IPTG+X-gal supplemented agar plates while pMALc-NN-EGFP and pEB13-lacZNs₅A/B carrying cells formed blue colonies, indicating that co-expression of MBP-scNS3 with the engineered β-galactosidase results in cleavage of the engineered enzyme.

TABLE 1
β-galactosidase activities directed by various constructs

| Construct | β-galactosidase units* |
|---|---|
| pEB13-/αcZ | 1057 |
| pEB13-/flcZ-NS5A/B | 130 |
| pEB13-/αcZ-NS5A/B + pMALc-NN-EGFP | 80 |
| pEB13-/acZ-NS5A/B + pMALc-NN-\hat{ }NS3 | 20 |
| pMGT14 | 60 |
| pMGT15 | 160 |

β-galactosidase units*: Miller units as described (Bach et al., 2001). The presented units are an average of 3 independent assays, each in triplicate.

Next, the MBP˜,ycNS3 was subcloned under the control of the araBAD promoter that resulted in plasmid pBAD-MBP-J CNSS. The purpose of this transaction was to allow the use of different inducers for NS3 (arabinose) and for potential inhibitors that are expressed from the pMALc-NN platform (Bach et al., 2001) that utilizes the strong, IPTG inducible tac promoter. TG-I cells carrying ρBAD-MBP-,ycNS3 and pEB13-lacZNssA_{/B} formed white colonies on arabinose+X-gal supplemented agar plates while pBAD-MBP-ˆNS3ₘᵤₜ (inactive NS3) and pEB13-lacZNs₅A/B carrying cells formed blue colonies on those plates. Finally both components, enzyme and substrate, were combined on a single plasmid, pMGT14 (SEQ ID NO:67). TG-I cells carrying pMGT14 (Test bacteria) formed white colonies on arabinose+X-gal supplemented agar plates while pMGT15 (inactive NS3) carrying cells formed blue colonies on those plates (FIG. 1A). For a quantitative analysis, test bacteria expressing active or inactive NS3 were induced with varying concentrations of arabinose and the β-galactosidase activity was measured (FIG. 1B). As shown, the efficiency of substrate cleavage was arabinose dose-dependent to some extent. However, cleavage was efficient even at the lowest concentration of inducer, in accordance with the design of a system where the enzyme (scNS3) concentration will surpass that of the substrate (encoded by the lacZNs₅A_{/}B gene). The time course of the NS3 catalyzed cleavage was analyzed in an immunoblot (FIG. 1C). As shown, the intracellular concentration of MBP-.scNS3 (lower arrow) increased over time of induction that corresponded to the extent to which the engineered β-galactosidase (upper arrow) was cleaved (lanes 1-6). No cleavage was evident in cells expressing the inactive NS3 mutant (lanes 7-9). The middle band is an irrelevant E. coli protein that is recognized by the polyclonal serum. As shown (lanes 1-6), the reaction proceeded to near completion, depleting β-galactosidase almost entirely over time. This was surprising considering that NS3 is subject to product inhibition and does not deplete the substrate when assayed in vitro (Berdichevsky et al., 2003). The cleavage of the lacZNS₅A/B (mw of 127 kDa) between residues 279-280 was supposed to yield products of 41.7 and 84.9 kDa. However, such products could not be detected in any of the immunoblots. Since it is unlikely that the polyclonal antiserum will fail to recognize both cleavage products, one may conclude that, for a presently unknown reason, these products are degraded. This serendipitous discovery indicates an increased sensitivity and accuracy of the disclosed system, since it is no longer subject to product inhibition and all the recorded inhibition would result from the introduced potential inhibitor. Based on these results one can conclude that β-galactosidase level measured in the disclosed “test bacteria” is an indicator for NS3 enzymatic activity level that can be detected by phenotypic changes on X-gal plates. This system may be adopted for other protease-substrate systems as long as the protease can be expressed in an active form in a host cell, preferably in bacteria.

Genetic screenings for proteases, for protease inhibitors in general (Dautin et al., 2000; Martinez et al., 2000) and for NS3 inhibitors in particular (Martinez and Clotet 2003) have been described but came short of providing the desired protease inhibitors. The present invention thus overcomes shortcomings of the previous methods.

### Example 2

### Application of the Genetic Screening for the Identification of NS3-Inhibiting scFvs

For the isolation of NS3 inhibitory antibodies, a diverse immune antibody phage display library of >10⁷ individual clones was constructed. The source of antibody genes was a pool of spleens from 3 mice that were immunized with NS3 and had serum titers >600000 after the third boost. A single cycle of DIP (Benhar et al., 2000) and of biopanning (Benhar and Reiter 2002) affinity selections of the phage antibodies were applied (separately) to partially enrich for NS3 binders. This was necessary to reduce the library size for screening a reasonable number of clones on indicative plates. The scFv clones obtained as affinity-selection output (about 10⁶ clones) were pooled and subcloned into pMALc-NN. The scFvs were tested for potential inhibition of NS3 in the form of MBP-scFvs since in that form the scFvs are stabilized as soluble active binders in the reducing environment of the cytoplasm (Bach et al., 2001). The pMALc-NN-scFv pool was introduced into pMGT14-carrying “test bacteria” resulting in a “bacterial genetic screening” carrying two compatible plasmids with three different regulatory mechanisms for expression: IPTG-inducible high-level scFv expression, arabinose-inducible medium level NS3 expression and constitutive low level of β-galactosidase expression. The system is thus tuned such as sufficient NS3 is made to fully digest the engineered β-galactosidase, unless inhibited by an excess of inhibitor that is produced at still a higher concentration.

The transformants (about 2×10⁶ colonies) were screened on X-gal indicative agar plates (FIG. 1D). A total of 100 blue colonies were picked and analyzed by BsfNl fingerprinting as described (Benhar and Reiter 2002) resulting in 25 clones being identified as unique. The residing pMALc-NN-scFv plasmids were recovered and re-introduced into “test bacteria” to validate the results of the initial screening. All 25 clones regained their blue phenotype confirming that the phenotypic change was, indeed a direct result of scFvs expression. A sample of four of the clones (10, 11, 35 and 171) is shown in FIG. 1E in comparison to four control scFvs, three of which (37Y, 44Y and 53Y) that were isolated from a human synthetic antibody library (Zemel et al., 2004), bind NS3 without inhibiting it and one (Anti Tac, directed against the human IL-2 receptor) totally irrelevant (Benhar, et al., 2000).

### Example 3

### Characterization of Specificity and Binding Affinity of Selected scFvs

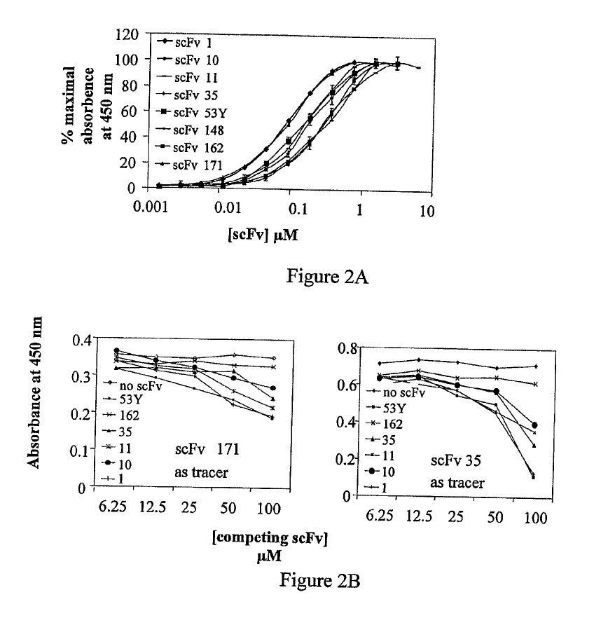

The scFvs isolated as blue colonies in the bacterial genetic screening were expressed and purified as soluble proteins and evaluated for scNS3 binding in an ELISA. Binding affinities were estimated from the half maximal binding signal. As shown in FIG. 2A, affinities of the selected scFvs and the control scFv 53Y (Zemel et al., 2004) ranged between 80 nM and 300 nM which are moderate binding affinities. The scFvs did not react with a panel of irrelevant proteins serving as specificity controls. The remaining 18 scFvs were weaker binders or weaker inhibitors in the quantitative assay (see below) and were not studied further. Thus, the disclosed genetic screening is very sensitive in identifying potential inhibitors that would escape detection by in vitro approaches. This is probably due to the MBP-fusion technology of scFv expression where the very high intracellular concentration of MBP-scFvs may compensate for its weak affinity (Bach et al., 2001).

To evaluate whether the scFvs NS3 epitopes overlap a competition ELISA was performed. As shown in FIG. 2B, scFvs 1, 10, 11, 35, 53Y and 171 could compete with scFvs 171 and 35 for specific binding to scNS3 while scFv 162 apparently did not. Since the tracers were the same scFvs with different epitope tags, relatively high concentration that prevented a full competition of binding had to be used. This is evident from the inferior inhibition obtained when the higher affinity scFv 171 is used as a tracer. Therefore, these data cannot be used to calculate binding affinities. Nonetheless, they do suggest that the binding epitopes of most of the identified scFvs overlap at least partially (probably located around the NS3 catalytic site). Interestingly, the control scFv 53Y (Zemel et al, 2004) which is non-inhibitory also competed the binding of both scFvs 35 and 171 to scNS3 (FIG. 2B). This highlights the advantage of using a screening for inhibitory antibodies that is based on actual inhibition rather than merely on antigen binding. Due to their large size (in comparison to small molecule inhibitors), scFvs may inhibit NS3-catalysis by blocking the access of the substrate to the active site without engaging it directly.

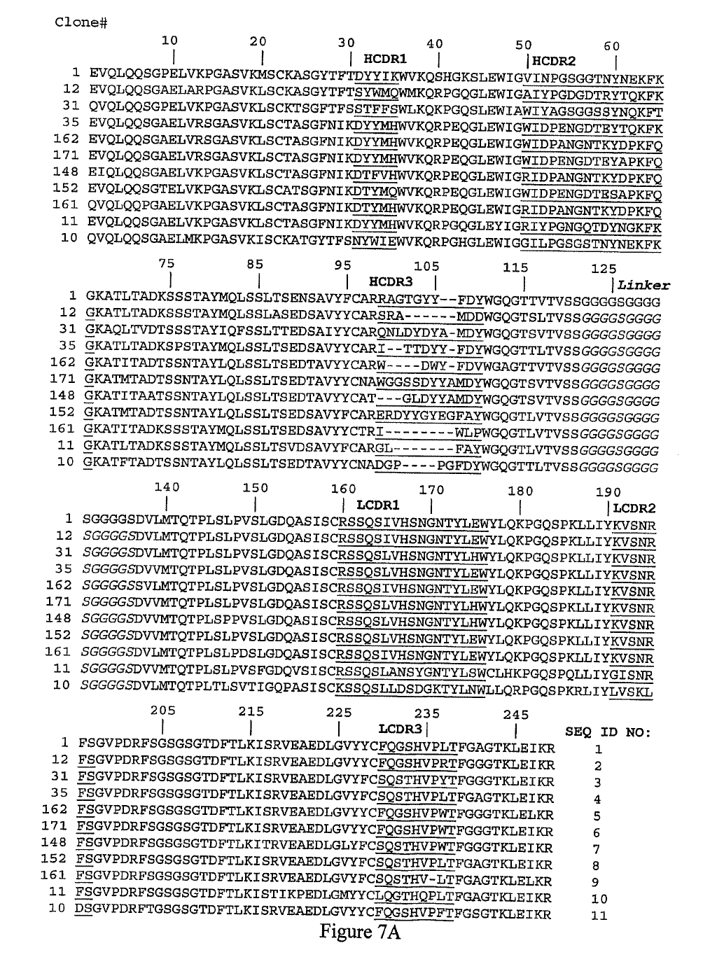

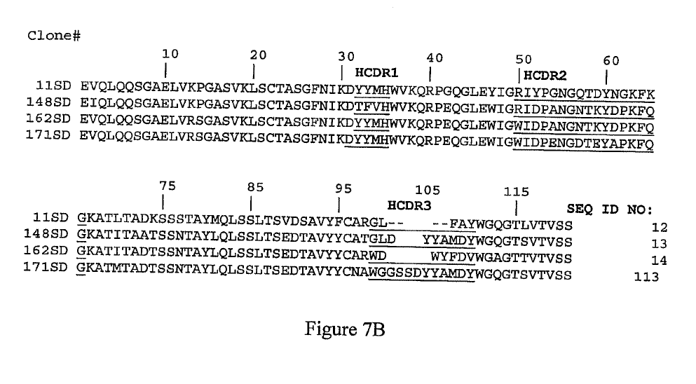

FIG. 7A illustrates the aligned amino-acid sequences of NS3-inhibiting scFvs (SEQ ID NOS: 1-11). The scFvs sequence analysis revealed a high degree of sequence identity in the V_{L} CDR sequences in contrast to a higher diversity observed in the V_{H} CDR sequences, particularly at CDR3. In contrast, a more limited repertoire of germline genes appears in the selected VH domains than in the V_{L} domains. Still, each V_{H} utilizes different D and J segments in the assembly of CDR3 while all the selected VLS utilize a single J segment, J5. This suggests that the V_{L} CDR3 was strongly selected by the disclosed genetic screening and is probably of critical importance.

FIG. 7B illustrates the aligned amino-acid sequences of four NS3-inhibiting single-domain antibodies (V_{H} dabs, SEQ ID NOS: 12-14). These clones were initially identified in the short dAb form due to internal stop codons or frameshifting followed by premature translation termination. They were also characterized after replacing the stop codon with a sense codon, or restoring the full-length ORF, as they appear with the corresponding number in FIG. 7A.

Table 2 summarizes the SEQ ID NOS of the scFvs and dAbs presented in FIG. 7, both as free recombinant antibodies and as their corresponding MBP fusion proteins.

TABLE 2
SEQ ID NOS of the scFvs and dAbs

| scFv | | scFv MBP | dAb | | dAb MBP |
|---|---|---|---|---|---|
| Clone | scFv SEQ | fusion SEQ ID | Clone | dAb SEQ | fusion SEQ ID |
| name | ID NO: | NO: | name | ID NO: | NO: |
| 1 | 1 | 15 | | | |
| 12 | 2 | 16 | | | |
| 31 | 3 | 17 | | | |
| 35 | 4 | 18 | | | |
| 162 | 5 | 19 | 162SD | 14 | 28 |
| 171 | 6 | 20 | 171SD | 113 | 114 |
| 148 | 7 | 21 | 148SD | 13 | 27 |
| 152 | 8 | 22 | | | |
| 161 | 9 | 23 | | | |
| 11 | 10 | 24 | 11SD | 12 | 26 |
| 10 | 11 | 25 | | | |

### Example 4

### Validation of the Selected scFvs as NS3 Inhibitors

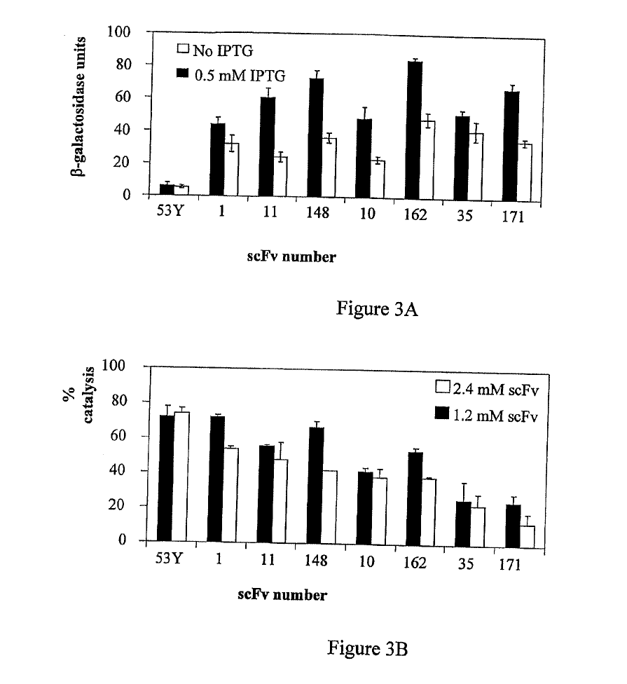

The potential of the selected scFvs to inhibit NS3 catalysis was quantitatively evaluated by measuring the β-galactosidase level that serves as an indicator for NS3 enzymatic activity level in genetic screening bacteria. As shown in FIG. 3A, cultures of the selected scFvs had high levels of β-galactosidase activity for all the scFvs tested, as compared to the control non-inhibitory scFv 53Y. The levels of β-galactosidase activity were in correlation to the level of scFvs expression, but could be detected even without IPTG induction due to the leakiness of the Ftac promoter under the assay conditions used. These results indicate that NS3 catalysis is inhibited by the selected scFvs in a dose dependent manner.

To further confirm the scFvs inhibitory effect on NS3 catalysis an in vitro fluorometric assay was applied for the measurement of NS3 protease catalysis and inhibition. As shown in FIG. 3B, all the tested scFvs inhibited NS3 catalysis in a dose dependent manner as compared to the control, non-inhibitory scFv 53Y. Different inhibition levels could be detected for the different scFvs and NS3 catalysis ranged between 12% and 54%. These results validated that the selected scFvs that (initially identified as blue colonies in the bacterial genetic screening and later validated as specific NS3 binders) are genuine NS3 protease activity inhibitors. As shown in Table 3, the binding affinity of the scFvs largely correlated (with scFv #1 as an exception) with their potency of in vitro inhibition. However, the β-galactosidase activity levels follow a different trend, probably due to different levels of accumulation and different stabilities of the scFvs in the cytoplasmic milieu.

TABLE 3
scFvs potency in in vitro inhibition

| ScFv clone | Affinity | β-gal | % inhibition |
|---|---|---|---|
| 171 | 90 | 67 | 88 |
| 35 | 160 | 51 | 78 |
| 10 | 280 | 48 | 62 |
| 162 | 280 | 84 | 62 |
| 148 | 170 | 72 | 58 |
| 11 | 300 | 60 | 52 |
| 1 | 80 | 44 | 46 |
| 53Y | 160 | 6 | 26 |

Affinity: apparent kD in nM estimated from the binding curves in FIG. 2B; β-gal: β-galactosidase units taken from FIG. 3A; % inhibition: value were obtained by subtracting the % catalysis value shown in FIG. 3B from 100%.

One goal in isolating scFvs that inhibit NS3 is for their application as intracellular antibodies in vivo. Intracellular scFv antibodies (intrabodies) with inhibitory properties present a new class of neutralizing molecules with great potential for gene therapy (Marasco, 1997; Marasco, 2001). Indeed, anti HCV intrabody-based approached were suggested by studies where a scFvs that binds and inhibits the NS3 helicase was isolated using phage display technology (Sullivan et al., 2002; Tessmann et al., 2002). As for anti NS3 protease intrabodies, the inventors have recently reported the isolation of non-neutralizing anti-NS3 scFvs by antibody phage-display that as intrabodies partially inhibited NS3-mediated cell transformation by diverting NS3 from the cell cytoplasm to the nucleus (Zemel et al., 2004). Inhibition of NS3 catalysis by small molecule inhibitors has been a formidable challenge due to featureless appearance of the NS3 catalytic groove that results in weak binding. Antibodies may offer a better solution by forming a “clamp” that involves interactions with NS3 that are located away from the active site on one hand, and blocking or interfering with substrate binding on the other. In the disclosed in vitro assay, the scFvs inhibit NS3 at micromolar concentrations, thus they should probably be improved to make more potent inhibitors. The fact that their affinities lie in the low micromolar range as well suggest that they could be easily manipulated by approaches such as in vitro affinity maturation by phage display approaches (Benhar 2001) or by further exploiting the disclosed genetic screening towards such an end.

### Example 5

### An Expression System for Fusion-Stabilized Peptides

### Construction and Validation

The design for expression of fusion-stabilized peptides (peptide aptamers) was based on fusion to the E. coli maltose-binding protein (MBP). Two fusion strategies were designed, with peptides fused at the C terminus of MBP, or constrained in an internal permissive position of the protein (position 133 was chosen according to a literature survey of permissive insertion positions of MBP; (Martineau et al., 1996).

The expression platform was constructed as follows: C-terminal peptides were introduced by PCR using pMALc-NN (Bach et al., 2001) as template and recloning into the same backbone. The ligated vectors were introduced into the “bacterial test strain” and IPTG induction was used to overexpress the peptides. For the internal (constrained) position, two Sfil restriction sites were introduced into the pMALc-NN vector at position 133 of MBP. SBl has a non-palindromic cleavage site; therefore upon digestion the resulting sticky ends could be used for directional cloning of oligonucleotide duplexes.

Initially control constructs were made for validation of the functionality of the screening. The appearance of a blue colony was expected to be indicative of an NS3 inhibitory effect of the peptide. Indeed, when “product-like” peptide (EASEDVVC) was cloned at the C-terminus of MBP, the resulting colonies in IPTG-induced “test bacteria” were blue. In contrast, when the FLAG epitope (DYKDDDDK) at the same position, the colonies, as expected for a non-inhibitor, were colorless.

Using a library screening approach, it is considered impractical to screen entire libraries (of 10⁷-10⁸ clones) on X-gal supplemented indicative plates since roughly 10⁴ colonies can be visualized on a single plate making it necessary to inoculate and screen thousands of plates for every library. Therefore, the inventors took advantage of the fact that the “reporter strain” bacteria, when expressing the substrate while ,ycNS3 expression is repressed, express a low, but sufficient level of β-galactosidase activity for growth on lactose as a sole carbon source. However, when the β-galactosidase is cleaved by NS3, the β-galactosidase activity is mostly lost resulting in retarded growth on lactose. Thus, upon introduction of the peptide libraries into the “test strain” (expressing both substrate and enzyme), growth of the bacterial transformants on lactose-minimal medium resulted in the enrichment of inhibitor-expressing clones. Thus, the NNS 8 library of 10⁸ clones was subjected to such enrichment before the blue/white screening.

The results described above proved that the screening is appropriate for application with peptide-aptamers and has a built-in enrichment property allowing identification of potential binders without having to resort to affinity-selection schemes.

### Example 6

### Construction and Selection of Libraries of Random Fusion-Stabilized Peptides

Two peptide aptamer libraries were constructed. The first library, Rand3, consisted of three random residues fused at the C-terminus of EASEDVVC, (the NS5A/B site P side) creating non-cleavable peptides (due to the absence of the P′4 residue). This library was expected to yield mostly product-like or P site-like inhibitors and was built mainly to evaluate the performance of the screening. The second library was the NNS 8 library where random octapeptides were fused at the C-terminus of MBP. Both libraries were screened using the genetic screening. A third library where 8-mer peptides are inserted after residue 133 of MBP (as constrained peptides) was also created for evaluation by the genetic screening.

For screening, DNA was prepared from the Rand3 library stock and introduced into pMGT14-carrying genetic screening bacteria that were plated directly on X-gal indicative plates. The limited library size (a theoretical complexity of 8000 clones that were over-represented in the 10⁵ cones library) allowed direct screening.

DNA was also prepared from NNS 8 library stock and introduced into pMGT14-carrying genetic screening bacteria. The large library size prevented direct screening and required enrichment for inhibitors prior to screening. The enrichment by virtue of faster growth rate of NS3-inhibited clones (where β-galactosidase is intact and may ferment lactose) on lactose as a sole carbon source was carried out by using the resulting transformants (10⁹ cells) to inoculate 1 liter of lactose minimal medium, incubated at 30° C. for 24 hours. Blue colonies were selected and plasmid DNA was recovered. To validate the “blue” phenotype the selected clones from both libraries were re-introduced into fresh test bacteria where only clones that formed again blue colonies were carried on for further analysis. The clones that had been validated were further tested for in vitro inhibition of NS3 catalysis.

### Example 7

### Validation and Characterization of the Isolated Peptides

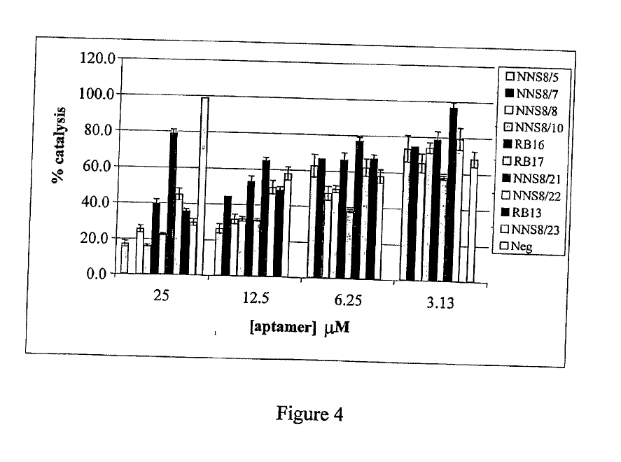

Several inhibitory peptide aptamers were so far identified and validated. Their sequences were determined from the expression plasmid (Tables 4 and 5). These plasmids were introduced into an appropriate bacterial expression host for overproduction of the peptide-aptamers. The purified aptamers were characterized for NS3 catalysis inhibition by an in vitro assay (Berdichevsky et al., 2003). As shown in FIG. 4, they were indeed inhibitors of NS3 catalysis with an IC₅₀ in the low micromolar range. However, their binding to NS3 could not be detected by ELISA.

TABLE 4

Deduced amino-acid sequence of the validated
peptide aptamers from the NNS8 library

| | | | Peptide | Peptide |
|---|---|---|---|---|
| | Peptide | | aptamer | aptamer |
| | amino | Peptide | (internal | (C′ |
| Clone | acid | SEQ ID | peptide) | peptide) |
| name | sequence | NO: | SEQ ID NO: | SEQ ID NO: |
| NNS8/N5 | LGLAVEVR | 29 | 36 | 43 |
| NNS8/N7 | FSRAEWFC | 30 | 37 | 44 |
| NNS8/N8 | GAALNTSS | 31 | 38 | 45 |
| NNS8/N10 | AVLAGLGV | 32 | 39 | 46 |
| NNS8/N21 | FWGGALWR | 33 | 40 | 47 |
| NNS8/N22 | CCAWLSIR | 34 | 41 | 48 |
| NNS8/N23 | LAAWFCLW | 35 | 42 | 49 |

Presented are the SEQ ID NOS of the free peptides and of the corresponding peptide apatamers comprising the peptides fused at the C of MBP or at an internal permissive position following residue 133 of MBP.

TABLE 5

Deduced amino-acid sequence of the validated
peptide aptamers from the Rand3 library
(weak inhibitors)

| | Peptide | | Peptide |
|---|---|---|---|
| | amino | | aptamer |
| Clone | acid | Peptide | (C′ peptide) |
| name | sequence | SEQ ID NO: | SEQ ID NO: |
| RB13 | EDVVCCWPV | 50 | 55 |
| RB15 | EDVVCCCLF | 51 | 56 |
| RB16 | EAVVCCLSL | 52 | 57 |
| RB17 | EDVVCCCGC | 53 | 58 |
| RB18 | EDVVCCVLY | 54 | 59 |

### Example 8

### Epitope Mapping of NS3-Inhibiting scFvs 35 and 171

As a potential source for information regarding contacts between the isolated neutralizing scFvs 35 and 171 to NS3, an epitope mapping procedure was used. A combined bacterial surface display of scFvs using the Lpp-OmpA system (Benhar et al., 2000) was used with random type 88 phage-displayed peptide libraries in which a second recombinant pVIII gene has been incorporated into the fd bacteriophage genome (Enshell-Seijffers et al., 2001). Two peptide libraries were used: a linear random 7mer library-FM7 (2χ1O⁹ clones) and a random library of 12 amino acids flanked by two constant cysteine residues thus generating random 12mer loops library—FDC 12C (5×10⁸ clones). Those two libraries were used to perform the Delayed Infectivity Panning-DIP selection (Benhar et al., 2000) in “reverse” where scFv-displaying bacteria were used to affinity select peptide-displaying phages. The affinity selected phages were used as an epitope-defining database that is applied via a computer algorithm (herein denoted TAU algorithm) that has been developed by the inventors (Enshell-Seijffers et al., 2003) to analyse the crystalline structure of the original NS3 antigen. On the basis of the predicted epitope, segment of the NS3 antigen was used to reconstitute an antigenic epitope mimetic that was examined as anti NS3 scFvs inhibition competitor. In addition, according to the predicted epitope point mutations were inserted into the NS3 antigen and those mutated antigens were validated for their antibodies inhibition activity. These procedures are detailed hereinbelow.

The bacterial surface display vector pIB-Tx (Benhar et al., 2000) which was used for antigen surface display through fusion of the antigen gene to the E. coli surface display system is compose of a chimeric Lpp-OmpA′ (Benhar et al., 2000). This vector was modified to enable cloning of the antibodies between the Ncdï and Notl restriction sites (Mazor et al., 2005). The new vector pIBTx-NN is used in this work to sub-clone the scFvs 35 and 171 from the pMALc-TNN vector through the Ncol and Notl restriction sites.

Next, spun-cell ELISA was used to examine the scFvs bacterial cell surface display efficiency and the ability of the cell displayed antibody to keep its functional specific binding to the NS3 antigen. Accordingly, induction conditions were determined, as well as the ability of cells displaying scFv-35 or 171 to bind MBP-,scNS3 antigen. As a negative control, anti CD30 scFv was cloned to the pIBTx-NN vector (Mazor et al., 2005).

The bacterial displaying plasmids (pIBTx-NN-scFvs 35 or 171) were introduced into E. coli TG-I cells. The cells were grown overnight in LB medium supplemented with 25 μg/ml kanamycin at 16° C. Cultures were diluted to OD₆₀oₙₘ of about 0.5 and IPTG was added to final concentration of 0.1, 0.01, 0.001 or OnM. The cells were further grown for 16 h and stored on ice. An aliquot equivalent to ImI of culture at OD₆₀oₙₘ of about 1.0 (˜10⁹ cells) was taken for analysis of surface display. Cells were collected by centrifugation and washed twice with ImI of PBST. Washed cells were then suspended in ImI of 5 μg/ml MBP-5cNS3 in PBST for Ih on ice. Next, cells were collected by centrifugation and washed twice with ImI of PBST. Then washed cells were incubated with a mixture of anti-MBP-,ycNS3 mouse serum (χ5000 dilution in PBST) and HRP-conjugated goat anti mouse antibody (×10000 dilution in PBST) for Ih on ice. The cells were washed twice with PBST and twice with PBS, by repeated centrifugation and re-suspension, and then 1 ml of the HRP substrate TMB was added. Color development was terminated with 0.5 ml H₂SO₄. Color was recorded at 450 nm.

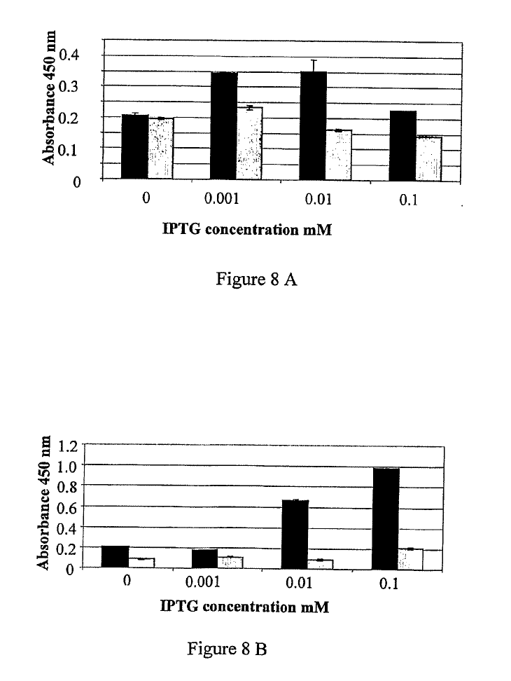

The results of the spun-cell ELISA are shown in FIG. 8. As shown, the best cell displaying scFv-171 was at 0.1 mM IPTG concentration, and for cell displaying scFv-35 the best IPTG concentration was 0.01 mM. ScFv-35 cell surface display appeared less efficient than scFv-171.

Next, the phage libraries were enriched for specific binding clones by subjecting the phages to two rounds of selection, reverse delayed infectivity panning (DIP) selection.

Bacterial display vector-containing cells (TG1-cells) were grown at 16° C., induced with IPTG and washed as for the spun-cell ELISA described above. The cells were re-suspended in 3% skim milk powder in PBS containing ˜10¹¹ cfu of peptide displaying libraries (FDC 12C, FM7 or mixture of both, wherein the two libraries were used separately for scFv-171 and the a mixture thereof was used for scFv-35) and left on ice for 1 h. The cells were then washed five times with PBST and five times with 2YT+kanamycin. Next, ImI of E. coli DH5αF′ cells at O.D600 of about 0.8 were added and the cells were shifted to 37° C. for 30 min without shaking followed by 30 min with shaking, then aliquots were taken for plating on tetracycline supplemented 2YT plates to obtain single colonies for screening and determination of phage panning output. Phage titration was done as described above. Phage stocks for the next DIP cycle were prepared by infecting 500-1000 ml DH5αF′ cells in 2YT medium with output phages. The infected culture was grown overnight at 30° C. and phages were prepared by centrifugation followed by PEG precipitation as described above.

As additional affinity selection to further enrich specific binders, phage particles were prepared from the second output library of scFv-35 reverse DIP selection, and applied to an affinity-selection by biopanning on MBP-scFv-35 antigen. A single well of a 24-well Cell Culture plate was coated with ImI of Mouse Monoclonal Anti MBP in PBS (χ2000 dilution) for five h at 4° C. Followed by one wash with PBS, ImI of 5 μg/ml MBP-scFv-35 in PBS was added for overnight at 4° C. After discarding the excess solution, the well was blocked with 3 ml of 3% skim milk powder in PBS for one h at room temperature. Next, the well was washed with PBS and ImI of 10¹¹ phage output from the second cycle of DIP selection on anti NS3 scFv-35 in 1.5% skim milk powder was added for one h with gentle agitation at room temperature. Unbound phages were rinsed away and the well was washed extensively ten times with PBST and ten times with PBS. Bound phages were eluted with ImI of 100 mM triethylamine pH 13.0 for 30 min at room temperature with gentle agitation. The eluate was transferred into 1.5 ml tube and neutralized with 200 μl of IM Tris-HCl, pH 7.4. To obtain single colonies for screening and determination of phage panning output, DH5αF⁺ bacteria were infected with the affinity selected phages, plated on LB plates containing tetracycline and grown at 37° C. overnight.

Following phage selection, randomly selected single clones that were picked from the second Reverse DIP and panning cycles, were screened for binding to anti NS3 scFvs 35 and 171 using immuno dot-blot analysis. 96 single colonies from output of scFv-171 reverse DIP with the FM7 library, 192 single colonies from FDC 12C library, 192 single colonies from scFv-35 reverse DIP with the mixed libraries and 192 single colonies from scFv-35 panning cycle were picked at random and phages were prepared in 96-wells plates as follows: single colonies were picked to 200 μl of 2YT+tetracycline in U-bottom 96-well plates. After overnight culture, the plates were centrifuged at 2100 rpm for 15 min. a portion (125 μl) of the supernatant from each well was transferred to a flat-bottom, 96-well plate already containing 50 μl/well of PEG/NaCl solution. The flat-bottom plates were incubated at 4° C. for two h and centrifuged. The precipitated phages were re-suspended in a total of lOOμl of TBS and applied via a vacuum manifold to nitrocellulose filters. After blocking with TBS/5% milk for one h, the filters were washed briefly with TBS and incubated overnight with selected MBP-scFv (50 μg/ml) in 2% milk in TBS at 4° C. with gentle rocking. After washing, the membranes were incubated with Mouse Monoclonal Anti-MBP (×10000 dilution in TBS) followed by Peroxidase-conjugated AffiniPure Goat Anti-Mouse IgG (×10000 dilution) in TBS/2% milk for one h at room temperature. The positive signals were detected by enhanced chemiluminescence (ECL) immunodetection. 14 suspected MBP-scFv-171 phage binders from 96 single colonies screening were picked for a verification Dot-Blot. Only one phage was verified as a binder at the verification Dot-Blot, although its dilutions look like it is inverted. The inverted dilutions could appear because of PEG remainders that interfere with the development of the color reaction.

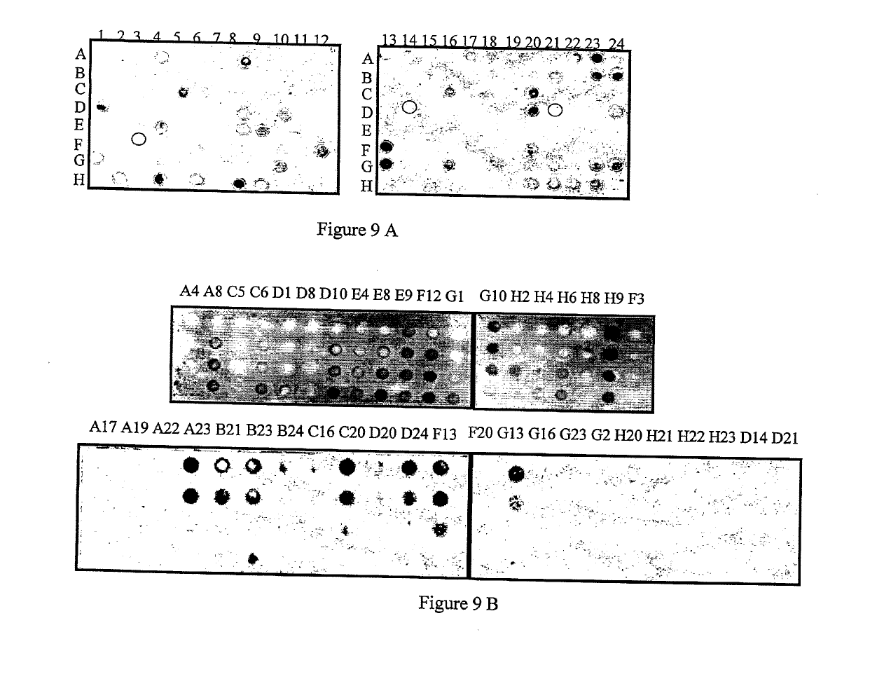

As shown in FIG. 9, 39 suspected MBP-scFv-171 phage binders from 192 single screened colonies were picked for a verification Dot-Blot. Here again, some inverted dilutions can be seen. Unbound phage that were taken from the first Dot-Blot screening as negative controls F3, D14 and D21 didn't bind the scFv-171 antigen at the verification Dot-Blot as well. 17 binders' were sequenced and analyzed by the TAU algorithm.

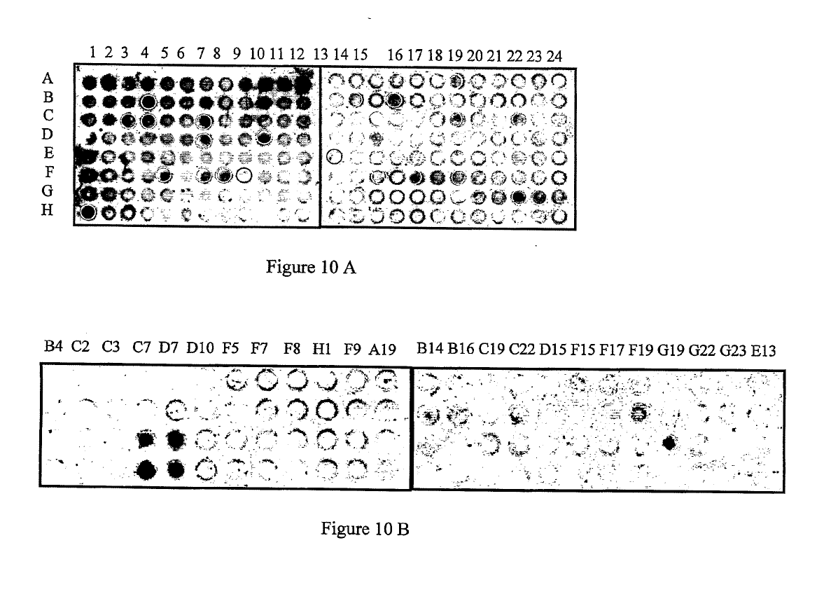

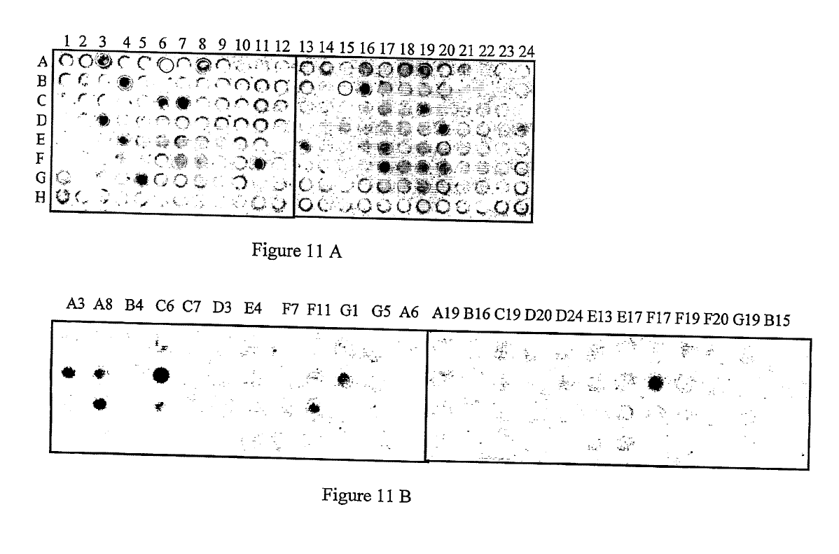

As for 35 binders shown in FIG. 10, 22 suspected MBP-scFv-35 phage binders from 192 single screened colonies were picked for a verification Dot-Blot. Here again some inverted dilutions can be seen. Unbound phages that were taken from the first Dot-Blot screening, as negative controls F9 and E13 didn't bind scFv-35 antigen at the verification Dot-Blot as well. Two binders were sequenced and analyzed by the TAU algorithm. In addition, from the panning output (FIG. 11), 22 of the 192 single screened colonies were picked and verified by another Dot-Blot. A6 and B15 were used as negative controls. From the positive binders, 8 clones were sequenced and analyzed by the TAU algorithm.

Sequences of peptides inserts of 17 scFv-171 positive binding phages (Table 10) and 10 scFv-35 positive binding phages (Table 11) were obtained.

TABLE 10
Sequences of the scFv-171 binders

| Clone | Peptide amino | Peptide |
|---|---|---|
| name | acid sequence | SEQ ID NO: |
| A8 | CCGGGWANNNPGNC | 83 |
| C6 | CCQTLQDHERKPEC | 84 |
| D10 | CSKMEKCTQNKLEC | 85 |
| E4 | CRHYRSYNYSRCSC | 86 |
| E8 | CDCKDSTKKGATSC | 87 |
| E9 | CGPGETCRKGIFHC | 88 |
| F12 | CSDCANADSLC | 89 |
| G10 | CITVQEMCDERDNC | 90 |
| H6 | CYCRKGTQSTHKAC | 91 |
| A17 | CAYHCATTRTFGAC | 92 |
| B13 | CAQQFEITNCRDQC | 93 |
| B21 | CTVSGSVCRYFWSC | 94 |
| C20 | CDRNTDSGRCGRVC | 95 |
| D20 | CRCIRPNNSPEGEC | 96 |
| D24 | CERGDLECGDTINC | 97 |
| F13 | CPMRSHSCNRVVHC | 98 |
| G13 | CTQIPEARACPWKC | 99 |

Table 10 presents sequences of inserts of 17 phages selected by screening of FDC 12C phage display peptide library with scFv-171. One of the selected peptides happens to contain only eleven residues.

TABLE 11
Sequences of the scFv-35 binders

| Clone | Peptide amino | Peptide |
|---|---|---|
| name | acid sequence | SEQ ID NO: |
| C7 | CILSISKCGSGVNC | 100 |
| D7 | CHPALC | 101 |
| A3 | CNCRSWQEMWDRAC | 102 |
| A8 | CAYPEGLSAGSEPW | 103 |
| F11 | CFYGVCIGTVDNRC | 104 |
| G1 | LRGVGGQ | 105 |
| G5 | CVASYLI | 106 |
| D20 | CVNPCSRSVETAIC | 107 |
| E17 | CGTRSSLCELQSRC | 108 |
| F17 | CRATTTPLTITPKC | 109 |

Table 11 presents sequences of inserts of 10 phages selected by screening of FDC 12C and FM7 mixed phage display peptide libraries with scFv-35. Two of the selected peptides are from the 7-mer FM7 libraries and one happens to contain only six residues.

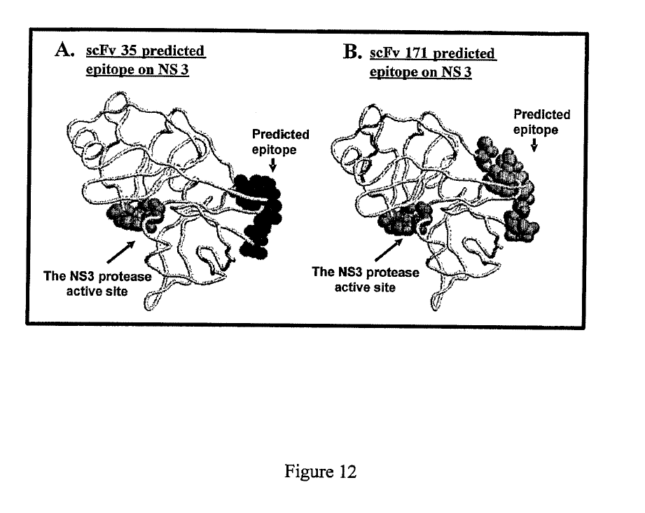

As shown, no obvious sequence identity exists between scFv171 positive binding peptides and between scFv-35 positive binding peptides. To solve this problem we used a systematic computer algorithm that enables us to focus on the common denominators of the peptides and use this information to map epitope onto the surface of the solved crystalline structure of NS3 protease domain: NS4A peptide complex (PDB-IA1R). As shown in FIG. 12, the two predicted epitopes, namely the scFv-35 epitope and the scFv-171 epitope on NS3 were mapped to the same region and shares seven identical residues. The epitope according to this prediction is far from the catalytic triad

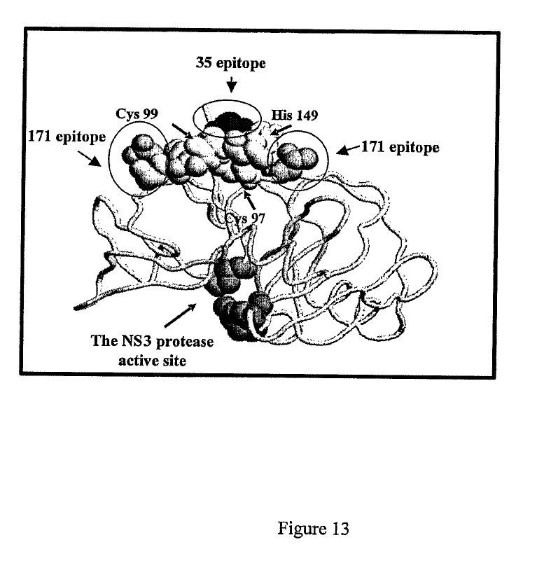

As shown in FIG. 13, scFv-35 and scFv-171 overlapping epitopes contain two cysteine residues that are part of the protease zinc-binding site. In addition scFv-171 epitope contains an additional residue of this binding site histidine 149.

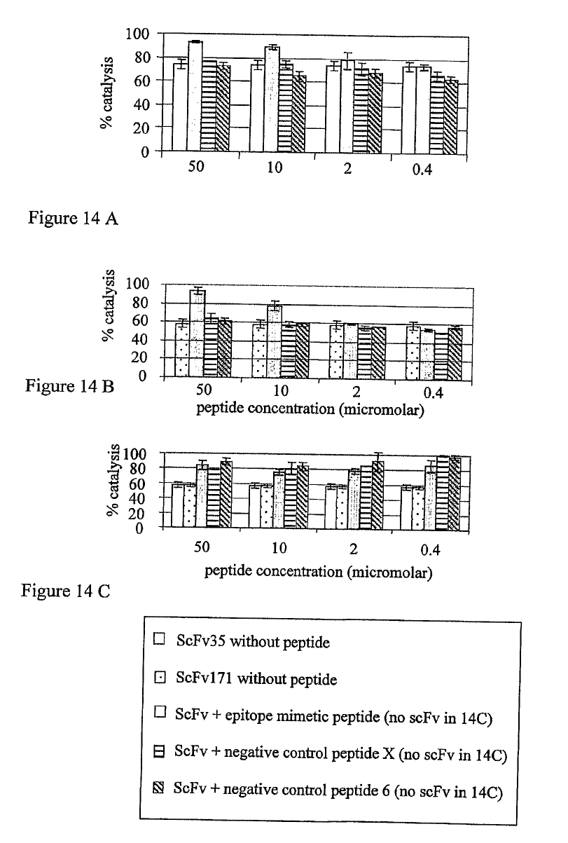

In order to confirm the prediction of scFv 35 and 171 shared epitope on NS3, the epitope was reconstituted on basis of the prediction. To that end, a short peptide segment of NS3 that was predicted to comprise the antibodies shared epitope was used to generate an epitope mimetic. A 14 amino acids peptide was designed, that appear as linear residues on NS3, residues 90-103 N′-GARSLTPCTCGSSD-C (SEQ ID NO: 110 which contains the shared residues of both epitope predictions (marked in bold letters). This epitope mimetic peptide was used in the in vitro fluorometric assay. The logic of this assay is that if this peptide represents a part of the true epitope, scFvs will bind to it, thus scFvs will be occupied and inhibition of NS3 catalytic activity will decrease, meaning NS3 activity will increase. In this assay scFvs were added at 200 μg/ml concentration, and peptides were added in ×5 dilutions starting at 50 μM. As negative controls, a peptide 21 amino acids in length denoted peptide 6 (N′-TLDPRSFLLRNPNDKYEPFWK-C, SEQ ID NO: 111) and a peptide eight amino acids in length denoted peptide x (N′-ISEVNLDA-C, SEQ ID NO: 112) were used. To test whether those peptides have any effect on NS3 catalytic activity a control in vitro assay was performed, in which the peptides and the NS3 enzyme were added alone without the inhibitor antibodies. The results are shown in FIGS. 14 A-C.

The results of this competitive in vitro assay (FIGS. 14 A-B) show a difference between the catalytic activities of NS3 with the presence of the peptide mimetic and without it or with control peptides, with both of the scFvs 35 and 171. In the scFv-35 competitive in vitro, adding the peptide mimetic increased NS3 catalytic activity in 20% from 73% to 93% catalysis, while in the scFv-171 competitive in vitro, adding the peptide mimetic increased NS3 catalysis in more than 30%, from 60% to 93% catalysis. Those values were received at the highest peptide dilution (50 μM). Catalysis did not reach to 100% because the proteolytic product inhibits the enzyme catalysis. These results suggest that the peptide may indeed represent a part of the NS3 epitope recognized by our antibodies. Accordingly, in the presence of the peptide mimetic scFvs are occupied binding it, therefore a decrease in NS3 inhibition can be seen. In addition, a control in vitro assay was performed (FIG. 14C), in which it was tested whether the peptides (the peptide mimetic and two control peptides) have any effect on NS3 catalytic activity. The results of this control assay show that none of the peptides by itself affected NS3 catalytic activity.

### Example 9

### Inhibition of HCV-RNA Replicons by the NS3 Inhibitors

For intracellular expression of the anti-NS3 protease single-chain antibodies in the cytoplasm of mammalian cells, the scFvs coding genes were subcloned as Ncol-Notl fragments, form plasmid pMALc-NN-scFv into the pCMV/myc/cyto-MBP vector (Shaki-Loewenstein et al, 2005). The replication inhibition efficacy of the NS3-inhibiting intrabodies was evaluated using the SEAP secreting replicon system (Yi et al., 2002; Bourne et al., 2005). The seven inhibiting intrabodies including the control scFv 53Y were introduced into these replicon-bearing Huh7 cells by transient transfection of the pCMV-MBP-scFv vectors using the Fugene reagent. SEAP activity secreted from the cells was measured over successive 24-h intervals. Transfection efficiency, observed by expression of EGFP, was determined as about 30% and values were corrected accordingly.

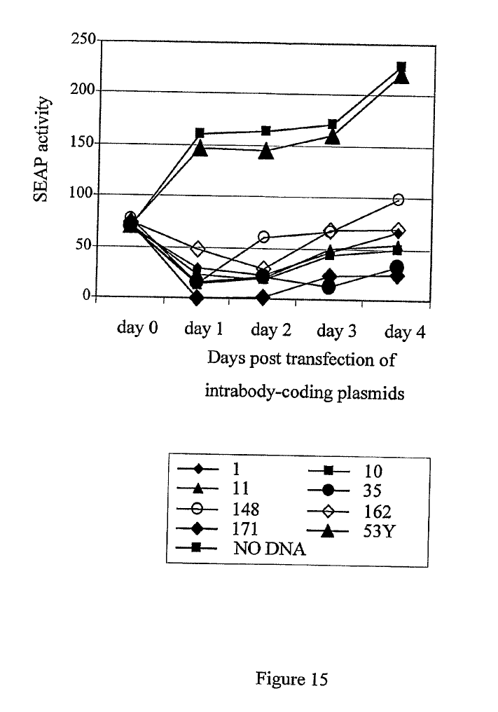

As shown in FIG. 15, SEAP secretion was inhibited in replicon cells transiently transfected with the NS3-inhibiting intrabodies but not with the control scFvs 53Y. All the seven intrabodies demonstrated inhibitory effect, ranging between 70-100%. Inhibition was well observed at the first and second days post transfection, when intrabodies expression level is high. At the third and fourth days post transfection SEAP secretion is rising, probably due to the decline in the expression of the transiently transfected intrabodies. Interestingly, scFvs 35 and 171 that were found to be the most potent inhibitors, as was observed in an in vitro assay, follow the same trend also in this assay, providing a strong validation for their inhibitory competence.

### REFERENCES

I. Zemel et al., 2001, J Viral Hepat 8(2): 96-102
2. Kwong et al, 1998, Antiviral Res 40(1-2): 1-18.
3. Martin et al., 1999, Protein Eng 12(11): 1005-11.
4. Lindenbach and Rice 2005, Nature 436, 933-8.
5. Hirowatari et al., 1993, Arch Virol 133(34): 349-56.
6. Hahm et al., 1996, Virology 226(2): 318-26.
7. Dautin et al., 2000, J Bacteriol 182(24): 7060-6.
8. Martinez et al., 2000, Antimicrob Agents Chemother 44(5): 1132-9.
9. Martinez and Clotet, 2003, Antimicrob Agents Chemother 47(5): 1760-5.
10. De Francesco and Migliaccio 2005, Nature 436, 953-60.
11. Baum et al., 1990, Proc Natl Acad Sci USA 87(24): 10023-7.
12. Cheng et al., 2004, Antimicrob Agents Chemother 48(7): 2437-47.
13. Berdichevsky et al., 2003, J Virol Methods 107(2): 245-55.
14. Sambrook et al., Molecular Cloning: A Laboratory Manual. 3rd edn., 2001, Cold Spring Harbor Laboratory Press.
15. Hoppe-Seyler and Butz, 2000, J Mol Med 78(8): 426-30.
16. Colas, 2000, Curr Opin Chem Biol 4(1): 54-9.
17. Bach et al., 2001, J Mol Biol 312(1): 79-93.
18. Martineau et al., 1996, Mol Immunol 33(17-18): 1345-58.
19. Zemel et al., 2004, J Hepatol 40: 1000-1007.
20. Marasco, 1997, Gene Ther 4(1): 11-5.
21. Benhar, 2001, Biotechnology Advances 19(1): 1-33.
22. Benhar et al., 2000, J Mol Biol 301(4): 893-904.
23. Peyman and Ulmann, Chemical Reviews, 90:1543-584 (1990).
24. Fingl et al, 1975, “The Pharmacological Basis of Therapeutics”, Ch. 1, p. 1.
25. Benhar and Engelberg-Kulka, 1993, Cell 72(1): 121-130.
26. Berdichevsky et al., 1999, J. Immunol. Methods 228(1-2): 151-62.
27. Feliu et al., 2002, Biochim Biophys Acta 1596(2): 212-24).
28. Marasco, 2001, Curr Top Microbiol Immunol 260: 247-70.
29. Sullivan et al., 2002, J Hepatol 37(5): 660-8.
30. Tessmann et al., 2002, Journal of Virological Methods 103(1): 75-88.)
31. Ausubel, et al., 1989, Current Protocols in Molecular Biology, Chapters 2 and 4 (Wiley Interscience).
32. Beerens, 2003, Curr Gene Ther 3(5):486-94.
33. Enshell-Seijffers, 2003, J Mol Biol 334, 87-101.
34. Enshell-Seijffers et al., 2001, Nucleic Acids Res 29, E50-0.
35. Shaki-Loewenstein et al., 2005, J Immunol Methods 303, 19-39.
36. Mazor et al., 2005, Mol Immunol 42, 55-69.
37. Yi et al., 2002, Virology 304, 197-210.
38. Bourne et al., 2005, Antiviral Res 67, 76-82.
39. Benhar and Reiter 2002, Current Protocols in Immunology, Chapter 10. 19B.

What is claimed is:

1. An isolated polypeptide or peptide comprising an NS3 inhibitor having an amino acid sequence as set forth in any one of SEQ ID NOS:1-49 and 113-114, or fragments, analogs, homologs, derivatives and salts thereof.

2. The polypeptide of claim 1, wherein the NS3 inhibitor has an amino acid sequence as set forth in any one of SEQ ID NOS: 1-14, 29-35 and 113.

3. The polypeptide of claim 1, wherein the NS3 inhibitor is fused to a stabilizing protein.

4. The polypeptide of claim 3, wherein the stabilizing protein is E. coli maltose binding protein (MBP).

5. The polypeptide of claim 4, selected from:
i) a polypeptide wherein the NS3 inhibitor is fused to the C terminus of MBP; and
ii) a polypeptide wherein the NS3 inhibitor is fused at an internal position following position 133 of MBP.

6. The polypeptide of claim 5 having an amino acid sequence as set forth in any one of SEQ ID NOS:15-28, 36-49 and 114.

7. A pharmaceutical composition comprising the isolated NS3 inhibitor of claim 1 and a pharmaceutically acceptable carrier or excipient.

8. An isolated nucleic acid sequence encoding the NS3 inhibitor of claim 1 as set forth in any one of SEQ ID NOS: 115-125, or fragments, analogs, homologs, and derivatives thereof.

9. A recombinant construct comprising an isolated nucleic acid sequence encoding the NS3 inhibitor of claim 1 operably linked to one or more transcription control elements.

10. A vector comprising the recombinant construct of claim 9.

11. A pharmaceutical composition comprising the vector of claim 10 and a pharmaceutically acceptable carrier or excipient.

12. A host cell comprising the vector of claim 10.

13. A pharmaceutical composition comprising the host cell of claim 12 and a pharmaceutically acceptable carrier or excipient.

14. A polynucleotide comprising a nucleic acid sequence encoding a recombinant reporter protein, wherein the reporter protein comprises a β-galactosidase derivative and an amino acid sequence comprising a protease recognition sequence between residues 279-280 of β-galactosidase, and wherein: (i) the recombinant reporter protein retains β-galactosidase activity; (ii) the cleavage of the reporter protein by the protease results in reduced β-galactosidase activity; and (iii) the cleavage of said reporter protein by the protease does not substantially result in accumulation of cleavage products capable of substantially inhibiting said protease.

15. The polynucleotide of claim 14, wherein the protease recognition sequence is an HCV NS3 cleavage site.

16. The polynucleotide of claim 15, wherein the NS3 cleavage site has an amino acid sequence as set forth in any one of SEQ ID NOS:60-63 and 70.

17. The polynucleotide of claim 16, wherein the recombinant reporter protein has an amino acid sequence according to any one of SEQ ID NOS:64-65.

18. A polypeptide encoded by the polynucleotide of claim 17.

19. A recombinant construct comprising the polynucleotide of claim 14 operably linked to one or more transcription control elements.

20. The recombinant construct of claim 19 further comprising a nucleic acid sequence encoding a protease capable of cleaving said reporter protein, operably linked to one or more transcription control elements.

21. The recombinant construct of claim 20, wherein the protease is HCV NS3 protease or a derivative thereof.

22. The recombinant construct of claim 21, wherein the protease has an amino acid sequence as set forth in SEQ ID NO:66.

23. The recombinant construct of claim 19 further comprising a nucleic acid sequence encoding a potential inhibitor of said protease, operably linked to one or more transcription control elements.

24. A vector comprising the recombinant construct of claim 19.

25. A vector according to claim 24 having a nucleic acid sequence as set forth in any one of SEQ ID NOS:67-69 and 82.

26. A host cell comprising the vector of claim 24.

27. A method of screening for protease inhibitors, comprising:
a) co-expressing a protease and a recombinant reporter protein cleavable by the protease in a host cell, wherein the cleavage of the reporter protein by the protease results in reduced activity of said reporter protein, and wherein the cleavage of said reporter protein by the protease does not substantially result in accumulation of cleavage products capable of substantially inhibiting said protease;
b) exposing the host cell to potential inhibitors of said protease; and
c) screening for host cells that retain said reporter protein activity.

28. The method of claim 27, wherein the recombinant reporter protein comprises a galactosidase derivative and a protease recognition sequence of said protease inserted in a permissive site of β-galactosidase so as to retain β-galactosidase activity.

29. The method of claim 28, wherein the protease recognition sequence is inserted between residues 279-280 of β-galactosidase.

30. The method of claim 27, wherein the protease recognition sequence is an HCV NS3 cleavage site.

31. The method of claim 30, wherein the recombinant reporter protein has an amino acid sequence according to any one of SEQ ID NOS:64-65.

32. The method of claim 27, wherein the protease is associated with a disease or disorder in a human or non-human subject, or wherein the protease is a viral protease.

33. The method of claim 32, wherein the protease is HCV NS3 protease, or wherein the protease has an amino acid sequence as set forth in SEQ ID NO:66.

34. The method of claim 27, wherein the host cell is a bacterial host cell.

35. The method of claim 34, wherein the host cell is E. coli.

36. A protease inhibitor isolated by the method of claim 27.

37. A method of treating Hepatitis C virus (HCV) infection or preventing the diseases and disorders caused by HCV infection in a subject in need thereof, comprising administering to the subject a therapeutically effective amount of a pharmaceutical composition comprising an NS3 inhibitor according to claim 1.

38. The method of claim 37, wherein said diseases and disorders are selected from the group consisting of hepatitis, cirrhosis, liver failure and hepatocellular carcinoma (HCC).

39. A method of treating Hepatitis C virus (HCV) infection or preventing the diseases and disorders caused by HCV infection in a subject in need thereof, comprising administering to the subject a therapeutically effective amount of a pharmaceutical composition comprising a recombinant construct comprising an isolated nucleic acid sequence according to claim 9, operably linked to one or more transcription control elements.

40. A method of treating Hepatitis C virus (HCV) infection or preventing the diseases and disorders caused by HCV infection in a subject in need thereof, comprising: a) obtaining cells from the subject; b) contacting the cells ex vivo with a therapeutically effective amount of a pharmaceutical composition comprising a recombinant construct comprising an isolated nucleic acid sequence according to claim 9, operably linked to one or more transcription control elements; and c) re-introducing said cells to said subject.

41. A method of preventing Hepatitis C virus (HCV) infection in a liver transplant, comprising: a) treating a liver transplant before transplantation ex vivo with a therapeutically effective amount of a pharmaceutical composition comprising a recombinant construct comprising an isolated nucleic acid sequence according to claim 9, operably linked to one or more transcription control elements; and b) transplanting the liver transplant to a subject in need thereof, thereby generating an HCV-immune liver transplant.
